(19) United States
(12) Patent Application Publication
(10) Pub. No.: US 2012/0095084 A1
(43) Pub. Date: Apr. 19, 2012

(54) MODULATION OF SEROTONIN NEURONS INVOLVING THE PET-1 TRANSCRIPTIONAL CONTROL REGION

(75) Inventors: Michael M. Scott, Boston, MA (US); Evan S. Deneris, Cleveland, OH (US)

(73) Assignee: Case Western Reserve University

(21) Appl. No.: 13/225,125

(22) Filed: Sep. 2, 2011

Related U.S. Application Data
(63) Continuation of application No. 11/263,941, filed on Nov. 1, 2005, now abandoned.
(60) Provisional application No. 60/624,141, filed on Nov. 1, 2004.

(51) Int. Cl. A61K 31/711; A61P 25/00; C12Q 1/68; C12N 15/85

(52) U.S. Cl. 514/44 R; 435/320.1; 435/6.1

(57) ABSTRACT

Serotonin neurons modulate most homeostatic Central Nervous System (CNS) functions while influencing the expression of behavioral traits such as mood, aggression and anxiety. Serotonin neuron dysfunction has been implicated in depression, addiction, autism and sudden infant death syndrome. This disclosure describes a straightforward, highly reproducible method for genetically accessing serotonin neurons and a sub-population of intestinal enterocytes, in vivo, using BAC-based transgenes that can be constructed using simple subcloning procedures. Compositions described herein include these transgenes and methods for making and using them to create transgenic mouse strains and identify serotonin and intestinal enterocytes without immunostaining.

### CROSS REFERENCES AND RELATED APPLICATIONS

The application claims priority from U.S. Provisional Application Ser. No. 60/624,141 entitled “GENETIC MANIPULATION OF BRAIN SEROTONIN USING THE PET-1 TRANSCRIPTIONAL CONTROL REGION”, filed on Nov. 1, 2004, herein incorporated by reference in its entirety.

### GOVERNMENT INTERESTS

The United States Government may have certain rights to this invention pursuant to work funded thereby at Case Western Reserve University under grant from the National Institute of Health, Grant No. NIH62723.

### BACKGROUND

Serotonin (5-HT) is a neurotransmitter that controls a wide range of behavioral and physiological processes including cognition, circadian rhythms, and mood. Defective serotonin signaling has been implicated in numerous human behavioral disorders such as impulsive violence, anxiety, and depression. Very little is known about the genes involved in serotonin neuron development or how these genes function. It is also unclear how the proteins these genes encode interact with one another to govern serotonin signaling. These genes constitute a genetic program that operates to generate and maintain a certain number of serotonin-producing (serotonin) neurons that function to modulate neuronal circuitry throughout the central nervous system. These genes have the potential to be powerful therapeutic targets for the diagnosis and treatment of disorders involving serotonin signaling. However, to begin to investigate the genes that control the function of serotonin neurons, a method to genetically manipulate them while maintaining the genetic integrity of other nervous cells is desirable.

Serotonin neurons are generated in the ventral hindbrain through a complex developmental program. Neurochemical differentiation of these cells is followed by cell soma migration and the extension of either ascending or descending projections that form highly collateralized pathways and synapses that innervate nearly all cytoarchitectonic structures of the brain and spinal cord. However, the mechanisms underlying these processes, all of which represent general aspects of nervous system development and function, are not well understood at the molecular level.

The characteristics of serotonin neurons suggest that this cell population would be an experimentally rich model for investigating basic molecular principles of neuronal function. First, the axonal projections that these cells send out contact nearly all cytoarchitectonic structures of the brain and spinal cord and engage in synaptic and paracrine forms of signaling. Second, serotonin neurons receive abundant neuromodulatory information from other monoamine projection systems as well as glutamatergic and GABAergic innervation from various forebrain structures. Third, serotonin neurons have been implicated in modulating diverse behaviors and physiological processes such as learning and memory, mood, circadian rhythms, pain, locomotion, and various homeostatic functions. Fourth, without wishing to be bound by theory, it is believed that serotonin signaling is sub-optimal in numerous human behavioral and physiological disorders such as impulsive violence, anxiety, depression, addiction, autism and SIDS.

Similar to other neuronal cell types, studies focused on serotonin neuron development and function have been hindered by the small number and scattered distribution of these cells. Genetic tools specifically targeted to serotonin neurons would dramatically increase their accessibility by permitting expression of fluorescent probes and reporters, transsynaptic-tracers, temporally controlled recombinases, and light sensitive ion channels to alter serotonin neuron activity. Tagged, dominant negative, mutated or allelic (pathogenic and non-pathogenic) versions of proteins ubiquitously involved, for example, in synaptic signaling and plasticity could be introduced specifically into serotonin neurons. Thus, genetic manipulation of serotonin neurons and their projections would permit in depth general studies of neuronal function including how signal transduction pathways, protein trafficking and activity impact a wide range of physiological processes and behaviors.

Pet-1 is an early marker of serotonin neurons that encodes an ETS-containing transcription factor. ETS-containing DNA binding proteins have been identified in diverse species including Drosophila melanogaster, Caenorhabditis elegans, Xenopus laevis, and more than 30 different ETS-containing DNA binding proteins have been found in mammals. Several ETS-containing transcription factors are expressed the vertebrate nervous system and have been implicated in cellular processes, including, for example, the regulation of cell proliferation, cell type specific differentiation, programmed cell death and oncogenic specific differentiation. Expression of Pet-1 in the brain is restricted to developing and adult serotonin neurons, and it is expected that Pet-1 is a key determinant of serotonin neuron phenotype.

To determine whether Pet-1 is a serotonin neuron specific marker, Pet-1 null mice were generated. Analysis of these Pet-1 null animals at the cellular, molecular, and behavioral levels have shown that most serotonin neurons fail to develop in the brain Pet-1 null mice and the serotonin neurons that remain are defective. The lack of functioning serotonin neurons leads to very low serotonin levels in the developing and adult brain. Behavioral analyses of these animals indicate that defective development of serotonin neurons is followed by aggressive and anxiety-like behavior in adults. These findings indicate that Pet-1 is an important determinant of serotonin neuron identity and illustrate the existence of a Pet-1 dependent transcriptional program that selectively couples early steps in serotonin neuron differentiation to serotonergic modulation of behavior in adult mammals.

Further experimentation has shown that Pet-1 transcription is restricted to serotonin neurons and their post mitotic precursors in the developing and adult rodent brain. All serotonin-synthesizing neurons in the brain express Pet-1 and embryonic expression of Pet-1 precedes the appearance of serotonin neuron-specific traits. Pet-1 is unique as the earliest specific marker of serotonin neurons identified to date (Hendricks, 1999). These expression characteristics suggest that the Pet-1 locus would have utility in the design of serotonin neuron-specific genetic techniques.

Identification of Pet-1 specific transcriptional regulatory elements in mice and humans will permit identification of transcription factors that operate upstream of Pet-1. An understanding of the elements necessary for serotonin neuron specific transcription can in turn be used to create genetic cassettes for serotonin neuron specific exogenous expression of genes. Non-limiting examples of exogenously expressed genes include, reporter genes such as Green Fluorescent Protein (GFP), Enhanced Yellow Fluorescent Protein (EYFP), LacZ, and any other gene whose transcription product permits the identification and sorting of transformed cells, and temporally controlled recombinases such as CRE or FLP, which would catalyze the insertion or interruption of specific genes in these serotonin neurons. By coupling genetic manipulation and analysis to behavioral and physiological observation, it is expected to be possible to determine how individual genes effect serotonin production and, ultimately, behavior in model organisms.

In order to more fully understand the function of serotonin cells in the central nervous system, there remains a need for the development of genetic tools that allow the specific expression of genes in serotonin neurons.

### SUMMARY OF INVENTION

In one embodiment, the serotonin neuron specific transcriptional enhancer is the transcriptional enhancer of the mouse Pet-1 gene (ePet). The ePet comprises the intergenic DNA fragment located 5′ of the transcriptional start site of the Pet-1 gene. This enhancer fragment contains as yet undefined serotonin neuron specific transcription factor binding sites and may be between about 0 kb to about 40 kb in length. The presence of this fragment 5′ of the transcriptional start site of a gene drives expression of the gene specifically in serotonin neurons.

In another embodiment, the serotonin specific transcriptional enhancer fragment is the transcriptional enhancer of the human FEV gene (eFev).

In an embodiment, a gene may be coupled to the serotonin specific enhancer fragment. The gene may be a selective marker, non-limiting examples of which are fluorescent markers, GFP, EYFP, and Lucuferase, bacterial enzymes such as LacZ, antibiotic resistance genes including a neomycin, hygromycin, puromycin, or ouabain resistance gene and combinations thereof. In another embodiment, the gene coupled to the serotonin specific enhancer fragment may be a recombinase gene, non-limiting examples of which are serine or tyrosine recombinases, Cre, Lambda-Int, HP-1, XerD, Flp and combinations thereof.

In another embodiment, a serotonin neuron specific transcriptional enhancer fragment coupled to a gene creating a transgene cassette that is coupled to a genetic carrier is provided. Non-limiting examples of genetic carries include plasmids, BACs, and miniBACs or any molecule suited to delivering the transgene cassette to serotonin neurons in the target organism in such a way as to allow expression of the gene. Non-limiting examples of methods of delivering the combination to the target organism include: adding an embryonic stem cell to which a genetic carrier containing the transgene cassette has been delivered by for example but not limited to electroporation, microinjection, or viral infection into an early stage embryo followed by homologous or recombinase mediated recombination of the enhancer-gene containing DNA fragment with the embryonic stem cells chromosome to an early stage embryo of the target species and creating a virus that is capable of infecting the target organism whose genome includes the transgene cassette and administering the virus to the target organism.

In one embodiment, the transgene is expressed from a site on the chromosome outside of the Pet-1, FEV, or homologous locus, and in another, the transgene is expressed from within the Pet-1, FEY, or homologous locus, and in still another, the transgene is expressed extra-chromosomally.

Methods for using the invention are also provided. In one embodiment, a selective marker is coupled to a serotonin specific enhancer fragment, which may be used to isolate serotonin neurons based on their production of the selective marker from developing or adult brain. In one embodiment, the selective marker is LacZ and serotonin neurons are identified by 3-galactosidase activity. In another embodiment, the selective marker is a fluorescent marker and serotonin neurons are identified by their ability to fluoresce when irradiated with UV light. Such methods can be used to identify the location of serotonin neurons in the developing and adult brain, or to elucidate the neurons that are contacted by serotonin neurons and are subject to serotonin signaling.

In one embodiment, a pure population of serotonin neurons can be isolated from nervous tissue to create serotonin neuron cell lines for cell culture. For example, serotonin neurons expressing fluorescent markers may be isolated by fluorescence activated cell sorting (FACS). The ability to isolate serotonin neurons will allow the purification of serotonin neurons specific mRNA which can be used generate cDNA and protein expression libraries that represent the full compliment of genes expresses by either developing or mature serotonin neurons or to generate a serotonin neuron specific mRNA chip that can be used to identify changes in the expression patterns of serotonin neurons in response to administration of active agents.

In another embodiment, the full proteome of the serotonin neuron can be purified from isolated mature and embryonic serotonin neurons. 2-D gel electrophoresis, proteomic microarray, and gas chromatography/mass spectroscopy can be used to measure the compliment of proteins expressed by serotonin neurons, the specific protein-protein interactions that they undergo as well as their post-translational modification status.

In another embodiment, mRNA and protein analysis are employed in development of treatments by measuring changes in mRNA and protein production in response to treatment in developing and adult mammals. Treatments can include therapeutic agents, drugs, active agents, pharmaceutical compositions, and physical therapy. Exemplary methods may include methods of determining the effect of a therapy, such as an active agent, comprising expressing a marker in serotonin neurons or intestinal enterocytes, administering an active agent and monitoring the subject.

In a further embodiment, potential drug interactions with proteins expressed by serotonin neurons can be tested by assessing the binding of the drug to proteins in the serotonin neuron protein expression library.

In another embodiment, changes in serotonin neuron transcription as a result of administration of a candidate drug can be assayed by treating an animal with the candidate compound, isolating serotonin neurons, and purifying the mRNA from the serotonin neurons. Changes in mRNA expression can then be detected with a gene chip assay relative to control animals. The gene chip could contain cDNA from total brain, or a serotonin neuron specific gene chip can be developed to measure the transcriptional in genes expressed specifically in serotonin neurons.

In another embodiment, serotonin neurons expressing a selective marker can be used to study serotonin neurons in vivo. In one embodiment, the electrophysiology of these cells can be tested using as a non-limiting example patch clamp recording. The response of serotonin neurons to therapeutic agents can be tested using electrophysiological techniques, and these results can be coupled to the behavior of model organisms to derive a better understanding of how serotonin neurons effect behavior. Similarly, the response of other neurons that receive signals from serotonin neurons can be observed after the administration of a therapeutic agent.

In yet another'embodiment, candidate drug target genes can be validated using a ePet-recombinase lines to selectively delete the candidate drug target gene in serotonin neurons. The function of the serotonin neuron in the absence of the gene can then be analyzed using electrophysiological and behavioral methods in vivo.

In another embodiment, electrophysiological changes in mice lacking a candidate target gene can be monitored while simultaneously expressing a marker, such as EYFP, in serotonin neurons. The EYFP expression will facilitate identification of serotonin neurons in tissue slices used in electrophysiological methods.

In another embodiment, ePet-recombinase lines may be used to introduce a second copy of a candidate gene selectively in serotonin neurons, and the effect of overexpression of the gene product can be observed behaviorally and electrophysiologically.

In another embodiment, a 2 kb ePet enhancer is developed for use in viral transgene delivery in vivo and in brain tissue slices. The behavioral and electrophysiological effects of genetic manipulation selectively in serotonin neurons can then be assayed. Furthermore, a 2 kb ePet fragment can be used to create a transgene delivery system as a therapeutic vehicle for humans and livestock.

In further embodiments, the ePet enhancer can be used to modify the expression of the Pet-1 gene as a possible therapeutic target. ePet fragments can be coupled to a reporter gene such as luciferase and transfected into a model cell type in vitro. Changes in luciferase transcription would directly reflect the modulation of Pet-1 transcription as a result of the administration of putative therapeutic agents. The modulation of Pet-1 may be an important therapeutic goal as increased gene dosage of Pet-1 has been shown to elevate the levels of brain serotonin.

Serotonin neurons figure prominently in spinal cord injury research because these neurons appear to have significant regenerative capacity and they are critically important for functional recovery of movement. In one embodiment of the present invention, the use of ePet-fluorescent marker lines will allow researchers to easily visualize growth and activity in serotonin neurons when used in functional recovery tests. Fluorescent serotonin neurons would also facilitate investigations of the mechanisms that confer their regenerative properties.

Outside the CNS, ePet enhancer fragments have been shown to direct expression of markers in the cells of the pancreas and intestinal brush boarder epithelial cells. Pancreatic expression has been reported for other transgenes, however the intestinal expression appears to be unique. This suggests that the ePet enhancer fragment identifies a unique population of intestinal enterocytes. All of the purification, gene and protein array applications described herein for serotonin neurons can be applied to labeled intestinal enterocytes. These cells may perform a vital function in the intestine and may be a target for certain intestinal disorders. The ability to manipulate gene expression in these cells may be quite beneficial.

### DESCRIPTION OF DRAWINGS

The file of this patent contains at least one photograph or drawing executed in color. Copies of this patent with color drawings) or photograph(s) will be provided by the Patent and Trademark Office upon request and payment of necessary fee.

For a fuller understanding of the nature and advantages of the present invention, reference should be had to the following detailed description taken in connection with the accompanying drawings, in which:

FIG. 1. Development of the ePet method. A) Schematic illustrating the mouse BAC RP23-11D65 used to prepare transgenes. B) Two step procedure for preparation of miniBAC transgenes.

FIG. 2. Serotonin neuron-specific transgene expression in all nine adult serotonergic B nuclei serotonin neurons.

FIG. 3. 540Z expression localizes exclusively to serotonin neurons in B nuclei.

FIG. 4. The 540Z Transgene Recapitulates the Temporal Expression of the endogenous Pet-1 gene. Co-localization of serotonin with β-galactosidase.

FIG. 5. Cre Recombinase expression in developing and adult serotonin neurons. Co-localization of TPH and R26R conditionally expressed β-galactosidase, and co-localization of Cre recombinase with serotonin in rostral and caudal serotonin expression domains within the hindbrain of E 12 embryos.

FIG. 6. An ePet-Cre line showing delayed recombination of the R26R indicator allele in a subset of adult serotonin neurons.

FIG. 7. Electrophysiological properties of YFP-positive B9 neurons.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “gene” is a reference to one or more genes and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, particular methods, devices, and materials are now described. All publications mentioned herein are incorporated by reference in their entirety. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

“Optional” or “optionally” means that the subsequently described event or circumstance may or may not occur, and that the description includes instances where the event occurs and instances where it does not.

“Substantially no” or “substantially none” means that subsequently described event or circumstance occurs between in between about 0% and about 20% of non-targeted cells. More preferably, the event or circumstance occurs in between about 0% and about less then 5% of non-targeted cells.

Versions of the present invention include compositions and methods to readily and reproducibly drive gene expression specifically in serotonin neurons. As discussed above, Pet-1 transcription is restricted to serotonin neurons and their post-mitotic precursors in the developing and adult rodent brain. Pet-1 enhancer elements (ePet) located in the intergenic region of genomic DNA 5′ of the transcriptional start site of the Pet-1 ETS gene direct serotonin neuron specific expression of Pet-1. Therefore, ePet sequences can be used to drive expression exogenous genes in serotonin neurons by coupling the ePet enhancer sequence to these genes. ePet enhancer sequences may also be used to alter the expression of specific genes within serotonin neurons and modify protein expression specifically in these neurons altering their physiological state and, potentially, the physiological state of the other neurons that are contacted and influenced by serotonin neurons.

Transcriptional enhancer elements are cis-acting regulatory sequences involved in the transcriptional activation of eukaryotic genes. They function either 5′ or 3′ of the transcription start site, may be present in either orientation, and can be directly adjacent to or far from the transcription start site. Enhancer elements are composed of DNA binding sites for transcription factors that activate transcription. Once bound to an enhancer element activating transcription factors recruit other transcription factors and components of the RNA polymerase complex to the transcription start site or work to open the chromatin structure making it accessible to the RNA polymerase machinery. In this way, the binding of specific transcription factors to enhancer elements initiates or boosts transcription of the genes whose transcription they regulate. The ePet enhancer has a unique combination of transcription factor binding sites that initiate transcription specifically in serotonin neurons.

Non-limiting examples of the present invention include compositions that include transcriptional enhancer elements of murine Pet-1 (SEQ ID NO: 10) (ePet) and human FEV (SEQ ID NO: 11) (eFev) provided vide infra, and homologues and variants thereof. The enhancer sequence may be orientated 5′ to 3′, and the Pet-1 and FEV enhancer elements terminate at the translational start site. The sequence GATAAGAGGGGC is part of the promoter and is at the end of both the mouse and human enhancer sequences. Without wishing to be bound by theory, this sequence may be a recognition sequence for components of RNA polymerase that bind to and begin transcription of the Pet-1 and FEV genes. Transcription factor binding sites that may be responsible for driving gene expression in serotonin neurons are contained within the intergenic region 5′ to this sequence and make up the serotonin specific enhancer. The N's in the human eFEV sequence (SEQ ID NO:11) correspond to gaps where the sequence is still unconfirmed. Even where the sequence is still unconfirmed, one skilled in the art, could without undue experimentation, and using the methods and compositions of the present invention be able to prepare similar constructs and know that such constructs were active in serotonin neurons based on the expression of reporter proteins, serotonin neuron specific immunostaining, or by inactivation of floxed genes.

Due to the degeneracy of the genetic code, one of ordinary skill in the art will recognize that a large number of the nucleic acid molecules having a sequence at least 80%, preferably 85% or 90%, still more preferably 95%, 96%, 97%, 98%, or 99% identical to a nucleic acid sequence encoding the genes that comprise Pet-1 and FEV genes, ePet, and eFev enhancer sequences, or such genes or sequences linked to recombinase genes, reporter genes, and other gene sequences that express polypeptides in serotonin neurons or intestinal enterocytes can be prepared and used based on this disclosure. It will be further recognized that, for such nucleic acid molecules that are not degenerate variants, a reasonable number will also encode genes and polypeptides that are expressed in serotonin neurons.

Variants included in the invention may contain individual substitutions, deletions or additions to the nucleic acid or polypeptide sequences of transcriptional enhancer sequences or proteins that can be transcriptionally active (enhancers and promoters) or expressed (proteins) in serotonin neurons or intestinal enterocytes. Such changes will alter, add or delete a single nucleic acid in the case of transcriptional enhancer variant or amino acid in the case of protein variant or a small percentage of amino acids or nucleic acids in the encoded sequence. Variants of the present invention may include those that are referred to as “conservatively modified variants” where the alteration results in the substitution of an amino acid or nucleic acid with a chemically similar amino acid or nucleic acid.

Homology refers to similarities in DNA or protein sequences between individuals of the same species or among different species. In comparing nucleic acid sequences, the extent to which two nucleic acid sequences have identical bases at equivalent positions, can be expressed as a percentage. Homologous transcriptional enhancer sequences and variants of these from other animals that result in the expression of proteins and genes in serotonin neurons or in intestinal enterocytes can be used in the compositions and methods of the present invention. For example, the 40 kb transcriptional enhancer fragment from a mouse may be replaced with the homologous 60 kb human transcriptional enhancer fragment, a variant of the homologous 60 kb human transcriptional enhancer fragment, or a variant of the 40 kb human transcriptional enhancer fragment for expression of nucleic acids and proteins in serotonin neurons or intestinal enterocytes. Homologous transcriptional enhancer sequences and their variants that selectively express genes and proteins in brain serotonin neurons and select intestinal enterocytes may be used in the practice of the present invention. Suitable homologues to Pet-1, and ePet enhancers can also include those from vertebrates such as birds and fish. Both the bird and fish have a homologues of the Pet-1 gene. It would be reasonable to expect that fish, bird and other vertebrate sequences homologous with ePet transcriptional enhancer sequences and that express proteins in serotonin neurons could be used in the compositions and methods of the present invention.

Isolated ePet or eFEV transcriptional enhancer sequences can be coupled to eukaryotic or prokaryotic protein coding genes using restriction digestion and ligation, homologous recombination, or another method of joining specific DNA sequences. Expression of genes coupled to ePet enhancer elements selectively occurs in serotonin neurons with little ectopic expression. The serotonin neuron specific expression of LacZ is highly reproducible among different cell lines that have been transformed with LacZ under control of the ePet or eFev enhancer elements, which indicates that this enhancer fragment or variants and homologues thereof can be used for reporter gene and recombinase expression. The development of serotonin neurons that specifically express markers like GFP or other fluorescent markers would permit sorting these cells and then their use for studies in culture, preparation of serotonin neuron specific cDNA libraries, development of probes for microarray assays, and proteomic studies of serotonin neurons. By coupling these approaches to analyses of mouse behavior and physiological processes it is expected that it will be possible to determine how genes controlling serotonin neuron development and function including but not limited to mood, learning, memory and combinations of these in model organisms.

ePet sequences coupled to other genes may be prepared by methods including, but not limited to, ePet Bacterial Artificial Chromosome (BAC) transgenesis, miniBAC transgenesis, or Embryonic Stem cell (ES cell) knock-in approaches. Each of these can be used to exploit the important expression pattern of the Pet-1 gene for accessing serotonin neurons, in vivo. The ES cell knock-in approach can be used to generate animals with an exogenous or modified gene expressed from the Pet-1 locus. When using ES cell approaches it may be desirable to have at least one year of back crossing for studying the impact of a genetic modification on behavioral phenotypes. Genes may also be targeted to the Pet-1 locus in intact BACs by homologous recombination and introduced into mice and other mammals or vertebrates as a transgene. Large BAC clones that include all or most of the Pet-1 cis-regulatory elements may show improved transgene expression when compared to smaller BACs that may lack one or more ePet transcription factor binding sites. A large number of genes encoding a wide range of functionally diverse proteins can be genetically manipulated in serotonin neurons using the compositions and methods of the present invention. The utility of this technique can be extended to generate serotonin neurons that include exogenously expressed genes including, but not limited, to reporter genes, fluorescent markers, and detectable labels.

The present disclosure includes non-limiting examples of sequences that have been used to, or would be expected to, prepare transgenes containing ePet or eFev enhancer sequences and express various eukaryotic or prokaryotic proteins in mammalian or vertebrate embryonic or adult serotonin neurons. Enhancer sequences encompass the entire intergenic region 5′ to the Pet-1 or FEV fragments of this sequence, or truncations that were made from the larger fragments. Without limitation, useful fragments of this region may be between 2 kb and 40 kb in length or any variant or homologous fragment thereof. For example, the 2 kb fragment derived from the 40 kb mouse genomic fragment coupled to a reporter gene resulted in the expression of the gene and protein production in serotonin neurons. The sequence of the 2 kb fragment is therefore included in the large 40 kb fragment sequence. Shuttle vectors can be used to deliver a gene into the ePet or eFEV containing plasmid. Plasmids, including, for example, commercially available pBluescript from Stratagene, containing a LacZ or similar reporter gene, can be modified to include ePet or eFev enhancer elements. Alternatively, the LacZ sequence may be removed, to allow other genes including, but not limited to, recombninases such as but not limited to Cre, Flp, XerD, and HP1 and the like or reporters such as GFP, EYFP and the like, or antibiotic resistance genes to be substituted or included downstream of the beta-globin minimal promoter and the like. The recombinase or reporter gene/beta-globin minimal promoter construct can then be subcloned into a ePet containing miniBAC. In the present invention, the miniBAC vector backbone for the second construct is pBace3.6 a 8.7 Kb BAC vector that has previously been used to generate BAC genomic libraries.

There is a lack of ectopic expression in the brain for the majority of the ePet-LacZ lines tested. The 40 kb ePet mini BAC shows a very low level of both position effect and ectopic expression among different founder lines. This means that introduction of genes downstream of the 40 kb enhancer region with simple subcloning procedures can rapidly produce serotonin neuron specific transgenes and that each round of pronuclear injection has the potential to produce several desirable founder lines in the desired genetic background.

A modified BAC transgenic method can have advantages over well-established and widely used approaches in terms of generation time for each mammalian or vertebrate strain and specificity of serotonin neuron gene expression. In another embodiment, a genetics based method for serotonin neuron specific expression of transgenes in vivo may include the use of standard subcloning procedures to rapidly construct miniBAC transgenes, introduction of transgenes into genetically defined backgrounds, and generation of ePet miniBAC's with highly reproducible serotonin neuron specific transgene expression between different transgenic lines with little or no ectopic expression. This technique enables high throughput generation of mouse strains compared to existing approaches and therefore will increase the accessibility of serotonin neurons for molecular genetic, physiological, and behavioral studies. It is expected that this approach can be used to establish serotonin neurons as a model neuronal cell type for general studies of nervous system development and function in a behaving animal or mammal or vertebrate such as a human.

Studies of serotonin neuron function and physiology using ePet miniBAC serotonin neuron specific transgenes will largely eliminate the accessibility problem for serotonin neurons that arises from their small numbers and scattered distributions among numerous other neural cell types. It is expected that the ePet miniBAC transgene can be used to manipulate the entire Central Nervous System (CNS) serotonergic neurotransmitter system. By changing the gene coupled to ePet, expressed reporter proteins within the brain can be used by investigators to probe the entire serotonin system (serotonin neuron location, function, and response) in a variety of mammals. In one embodiment of the invention, transgenic cell lines are produced by creating a BAC that contains a fluorescent marker protein, such as EYFP, that is inserted into the Pet-1 locus by homologous recombination. Serotonin cells that have received the EYFP gene and fluoresce distinguishing themselves from other neuronal cells.

In another embodiment of the present invention, a BAC contains recombinase gene whose expression is directed by the ePet enhancer DNA fragment. The recombinase may be expressed in serotonin neurons where it will coordinate the insertion of an attachment site flanked transgene into genes on the chromosomes of transformed cells. BACs containing recombinase genes, such as but not limited to Cre, Lambda-Int, HP-1, XerD, and Flp, can be used for the delivery of transgenes, including but not limited to exogenously expressed genes, reporter genes, fluorescent markers, and other detectable labels, or selectively knock-out genes that contain recombinase attachment sites. Alternatively, the eFev enhancer sequence may be used to drive expression of the recombinase. Because mammals and other vertebrates are so closely related, it is reasonable to expect that an enhancer fragment from one species would work when injected into another. For example, it is reasonable to expect that the mouse ePet enhancer fragment could probably drive LacZ expression in humans.

Using ePet enhancer elements or variants or homologues thereof coupled to other genes, serotonin neurons may be isolated, free of other cell types, from embryos and adult mammals or vertebrates and especially mice. From this population of pure serotonin neurons, various genomic and proteomic array analyses may be performed. This approach may then be applied to examine cellular changes in numerous situations where the function of serotonin neurons is experimentally altered. For instance, the administration of commonly prescribed anti-depressants is thought to produce transcriptional changes in serotonin neurons. By administering these anti-depressants to the intact mammal or vertebrate that contain a transgene expressing a serotonin neuron specific marker, the serotonin neurons could be isolated and or cultured allowing the transcriptional changes that might occur to be accurately monitored. These results can be used to modify or study for example, drug structure function relationships, drug interaction, and optimum drug dosage.

In one embodiment of the present invention, ePet-Cre lines can be used to conditionally inactivate floxed genes and can be used to develop serotonin neuron specific genetic switches for temporal control of gene expression using either the tet on/off based systems or the tamoxifen-sensitive estrogen receptor. ePet-recombinase lines may also be used to screen prospective drug target genes by selectively removing the candidate drug target gene from the serotonin neurons. The function of serotonin neuron in the absence of this gene can then be analyzed by electrophysiological and behavioral methods in vivo. Alternatively, the effect of over expression of candidate drug targets can be tested by inserting additional copies of the target gene into the mouse genome and driving serotonin specific expression of the gene using the ePet enhancer sequence.

In another embodiment, ePet-EYFP lines can be used to FACS purify serotonin neurons from developing and adult brain. mRNA from these isolates serotonin neurons can be used to generate cDNA and protein expression libraries that represent the full complement of genes expressed in either developing or mature serotonin cells. This information can be used in the in the preparation of serotonin neuron specific microarray probes and will permit identification of transcriptional cofactors that operate with Pet-1 using serotonin neuron specific cDNA libraries for two hybrid assays.

In a further embodiment, 2-D gel electrophoresis, proteomic microarray analysis, or gas chromatography/mass spectroscopy analysis of serotonin neurons isolated using the compositions of the present invention may be used to measure the compliment of proteins expressed by serotonin neurons. Such an analysis would allow for the determination of specific protein-protein interactions as well as a description of the post-translational modifications status of serotonin neuron proteins. This information can be used in the development of pharmaceuticals, purified proteins, or peptidomimetics for the treatment and diagnosis of dysfunctional mammalian or vertebrate serotonin neurons, and the protein expression library can intern be used to assess the ability of these pharmaceuticals, proteins or peptidomimetics to bind serotonin neuron proteins. Moreover, microarray analysis techniques can be used to examine multiple genetic and proteomic interactions simultaneously. With the knowledge provided by these analyses, single changes to the genomic and proteomic complement of the serotonin neuron can be made using the compositions and methods previously described herein.

In another embodiment, mRNA purified from isolated serotonin neurons can be used to develop serotonin neuron specific gene chip which will allow the measurement of transcriptional changes in genes normally expressed by serotonin neurons. Such a gene chip can be used to determine the drug-mediated changes in serotonin neuron transcription by comparing mRNA expression of normal serotonin neurons and those of animals treated with the drug.

In another embodiment, ePet-EYFP lines can be sorted and the protein expression pattern of serotonin neurons can be derived. Proteins that can be used to monitor the activity or modulate the excitability of serotonin neurons can then be identified, and these monitors and modulators could be used to investigate serotonin neuron function in mammals and other vertebrates in response to putative serotonin neuron specific drug treatments in the developing and mature brain.

In yet another embodiment, the ePet-EYFP line, or other similar lines based on ePet variants and homologues, may allow rapid and precise identification of serotonin neurons for in depth electrophysiological characterization using methods including but not limited to patch clamp recordings which is highly advantageous over the multi-step, error prone and time consuming retrospective immunostaining method currently used.

The development of 2 kb truncated versions of the 40 kb fragment that retains the ability to functionally drive serotonin neuron specific expression of proteins would be useful in the development of a viral transgene delivery system. Such a viral delivery system may be used to deliver transgenes to mice in vivo or to slices of murine, human or other vertebrate brain tissue. The development of a viral delivery system may also be useful in the development of a human or livestock transgene delivery system.

The 40 kb and 2 kb fragments ePet could also be used to screen novel therapeutics for their ability to alter the transcription of Pet-1, a possible therapeutic target. These fragments could be coupled to a reporter gene, such as but not limited, to luciferase and transfected into a model cell type in vitro. Changes in luciferase transcription as a result of drug treatment may function as a direct reflection of a novel therapeutics ability to modulate Pet-1 transcription. The modulation of Pet-1 transcription may be an important therapeutic goal, as increased gene dosage of Pet-1 has been shown to elevate the levels of brain serotonin.

The methods and compositions of the present invention will permit directed investigations of serotonin neuron development, function, and psychopharmacology using all of the available molecular genetics tools. This could lead to the identification of new therapeutic targets for future treatment of various mental health disorders especially in humans.

A variable level of gene expression may accompany transgenes that integrate as one or multiple copies. While transgene expression may not occur at levels comparable to that of the endogenous gene being expressed in serotonin neuron, given the low position effects seen with the ePet miniBAC, this expression may be easily controlled for by determining transgene copy number in different founders by Southern blotting and by real time PCR that can quantitate levels of transgene expression. In one embodiment of the instant invention, those lines whose transgene expression level is comparable to that of the endogenous gene being driven by ePet could be selected for particular experiments. For example, when it is desirable to investigate gene dosage effects on behavior or to increase expression of markers that may be expressed below the level of detection when using ES cell knock-in approaches.

Serotonin neurons figure prominently in spinal cord injury research because these neurons appear to have significant regenerative capacity and they are critically important for functional recovery of movement. In one embodiment of the instant invention, fluorescently labeled serotonin neurons are used to facilitate spinal cord injury research by allowing investigators to visualize growth and activity of these cells when used in functional recovery tests. These fluorescent cells would also facilitate investigations of the mechanisms that confer the regenerative properties of serotonin neurons.

Outside of the CNS, the 40 kb fragment directs expression to cells of the pancreas and intestine. While the pancreatic expression pattern has been reported for other transgenes, the intestinal expression appears to be unique. Less than 5% of the intestinal brush border epithelial cells are labeled with 40 kb transgene driven LacZ. Labeled cells appear to be restricted to crypt epithelial cells. More specifically, the intestinal enterocytes that normally express the endogenous Pet-1 gene. It is reasonable to expect that the transgene identifies a unique population of cells within the epithelia, and provides a means to manipulate it. With the exception of the electrophysiology, all of the purification, gene and protein array applications for drug discovery described herein for the serotonin system could be applied to the labeled intestinal cells. It is reasonable to expect that this population of cells performs a vital function in the intestine and may be a possible drug target for intestinal disorders.

Various aspects of the present invention will be illustrated with reference to the following non-limiting examples.

### Example 1

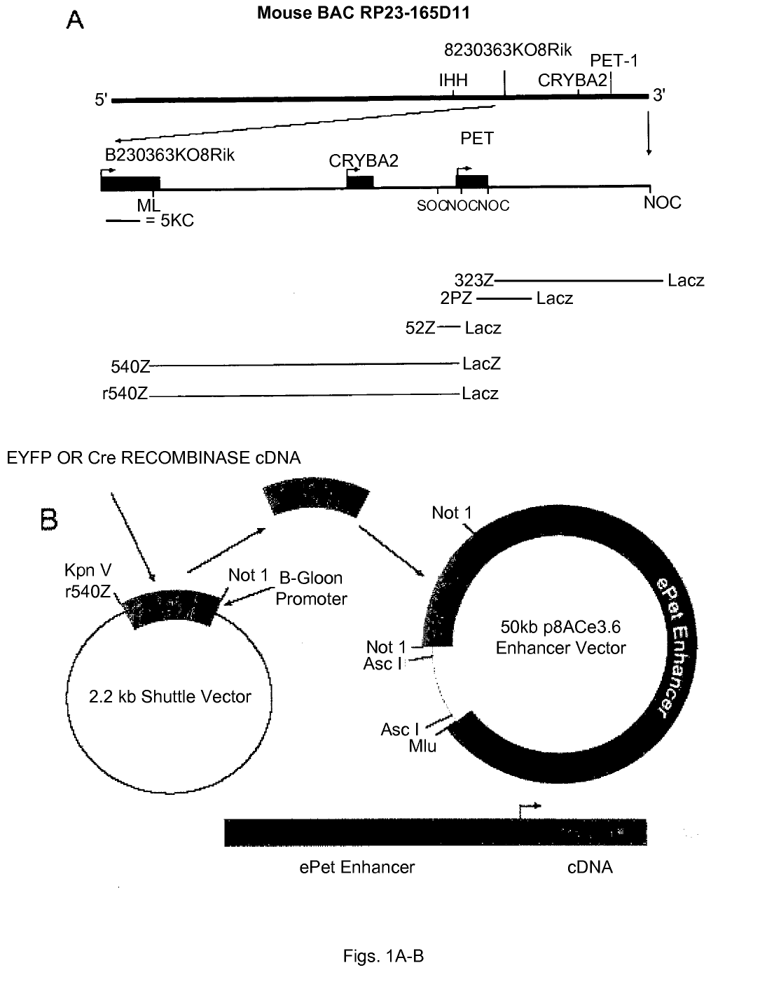

This example illustrates the identification of a genomic fragment in or near the Pet-1 locus which is used for driving reliable transgene expression in serotonin neurons. The analysis included genomic regions positioned either upstream, downstream or within the Pet-1 gene that were tested for transgene expression in serotonin neurons. Several mouse BACs were isolated and restriction mapped and restriction fragments for construction of five LacZ transgene reporters were selected, in which each fragment was positioned immediately upstream of the beta-globin promoter (FIG. 1A). Computer analysis suggests that the upstream genomic fragments carry a Pet-1 promoter region however a transgene with an upstream beta-globin promoter was prepared. Several transgenic founders were identified for each of the reporters and these founders were then bred to generate individual lines.

A bacterial artificial chromosome (RP23-165D11) containing the Pet-1 locus was isolated from the RPCI-23 C57BL/6 mouse BAC library by Research Genomics, using a probe synthesized from a genomic clone that had previously been shown to span the Pet-1 protein-coding region. BAC RP23-165D11 was then mapped using NotI, MluI and SpeI. Three oligonucleotide probes were used to identify by southern blot DNA fragments from this digest that were 5′ and 3′ of Pet-1, along with those that included the Pet-1 coding region: 5′ probe, 5′-GGG GTG GOC AAA GAT AAA G-3′, 3′ probe, 5′-ATC TGG GCC GAG GAT TTC-3′ and coding region probe, 5′-GCT ACG CCT ACC GCT TTG AC-3′. Four fragments were subsequently chosen to prepare LacZ transgenes, 52Z, 540Z, 2PZ, and 323Z (FIG. 1): a 2 kb SpeI/NotI fragment, a 40 kb MluI/NotI fragment composed of sequences extending upstream from the Pet-1 initiator methionine codon, a 2 kb NotI/NotI fragment composed of a portion of the Pet-1 protein-coding region, and a 23 kb NotI/NotI fragment composed sequences beginning immediately downstream of the last Pet-1 exon, respectively. The genomic fragments for 2PZ and 52Z were subcloned into a LacZ reporter plasmid, BGZA immediately upstream of its beta-globin minimal promoter in their natural orientation. Also included in this plasmid was an SV40 polyadenylation sequence. The 540Z, r540Z, and 323Z transgenes were built in the pBACe3.6 vector, a plasmid capable of supporting inserted fragments of large size with low levels of chimerism, and these transgenes were propagated as BACs. The polylinker of pBACe3.6 was modified such that its NotI sites were destroyed, the staffer fragment removed, and 5′-MluI, Not I, AscI-3′ added. The 40 kb fragment was then cloned into the MluI/Not I sites of the modified pBACe3.6. The BGZA 3′ polylinker was modified to contain KpnI, Eag I, Hind III sites to allow an Eag I digest to release beta-globin promoter, LacZ gene, and SV40 polyA sequences for subsequent subcloning into the pBace3.6 Not I site. The orientation of the 40 kb fragment relative to LacZ in pBace3.6 was determined by PCR, using primers, 5′-CTG CAG GCT AGA AGC AAA TG-3′ and 5′-GGG GTG GGC AAA GAT AAA G-3′, that annealed to the 3′ end of the 40 kb fragment and 5′ end of the beta-globin minimal promoter to produce a 600 bp fragment. The r540Z transgene carrying the 40 kb fragment in reverse orientation was also isolated from the same ligation by using the primers 5′-CGC TGT TTG GCT TGC TTT CTG AC-3′ and 5′-GGG ACT TGA GGC TGT GGC TTC-3′, product size of 430 bp, that annealed to the 5′ end of the 40 kb fragment and 5′ end of the beta-globin promoter. Cycling conditions for both primer sets were 94° C. for 180 s, 35 cycles of 94° C. for 30 sec, 58° C. for 30 sec, 72° C. for 60 sec, 72° C. for 300 sec.

The 323Z transgene was prepared by removing the b-globin, LacZ, and polyA sequences from BGZA with EagI and then subcloning this fragment into the modified pBACe vector, which regenerated the Not I site. Finally, the Not I/Not I3′ 23 kb genomic fragment was subcloned into the NotI site, with no attempt made to determine its orientation relative to the LacZ gene.

40 kb of mouse genomic DNA directly 5′ of the Pet-1 ETS transcription factor (40 kb fragment) can direct LacZ reporter gene expression to embryonic and mature serotonin neurons of the mouse. The homologous human gene fragment to Pet-1 can drive reporter gene expression in serotonin neurons of the mouse. The activity of the 40 kb mouse fragment has been reduced to 2 kb of genomic DNA directly 5′ of the pet-1 gene in the mouse.

TABLE 1
Transgene expression patterns

| Transgenic | # of | Embryonic | Adult | Adrenal | | | | |
|---|---|---|---|---|---|---|---|---|
| lines | Founders | 5HT | 5HT | medulla | RGC | Intestine* | Pancreas* | Ectopic |
| 540Z | 10 | +++ | +++ | − | − | +++ | + | + |
| | | (10/10) | (10/10) | (5/5) | (5/5) | (5/5) | (3/3) | (3/10) |
| r540Z | 6 | +++ | +++ | NT | NT | +++ | NT | + |
| | | (5/5) | (6/6) | | | (1/1) | | (3/6) |
| 52Z | 9 | +++ | +++ | NT | NT | +++ | NT | +++ |
| | | (1/1) | (7/9) | | | (1/1) | | (7/9) |

Reproducibility of transgene expression in developing and adult serotonin neurons and level of expression in non-serotonergic neuron cell types.
+++, strong LacZ expression;
+, weak LacZ expression;
− no LacZ expression detected.
Numbers in parentheses indicate the fraction of LacZ lines expressing in the indicated site.
*a small subset of enterocytes and pancreatic cells strongly expressed LacZ.
NT, not tested.
Transient 540Z expression was also consistently detected in a very small portion of embryonic skin beginning at E13 and ending by E17 (data not shown).

Serotonin neuron-directed mini-BAC transgene expression. Serotonin-synthesizing neurons are located in the midbrain and medulla and are organized into nine clusters termed the B nuclei. Some B nuclei are located in raphe positions while other have a scattered distribution that extends into the reticular formation. Transgene expression in serotonin neurons was not detected in any of several lines in which a downstream genomic fragment or Pet-1 gene fragment was placed upstream of the LacZ gene. In contrast transgene expression was detected in B nuclei of mice carrying the 5′ 2 kb reporter, 52Z. The majority of 52Z lines showed LacZ expression in both embryonic and adult serotonin neurons (Table I). These results indicate that transcriptional elements capable of driving transgene expression in serotonin neurons are located within a 2 kb region immediately flanking the Pet-1 protein coding exons. However, strong LacZ expression was also consistently seen in numerous ectopic cellular locations in and outside of the brain (Table 1). These findings indicated that the 52Z was incapable of driving serotonin neuron specific transgene expression and led us to test a larger fragment (FIG. 1A).

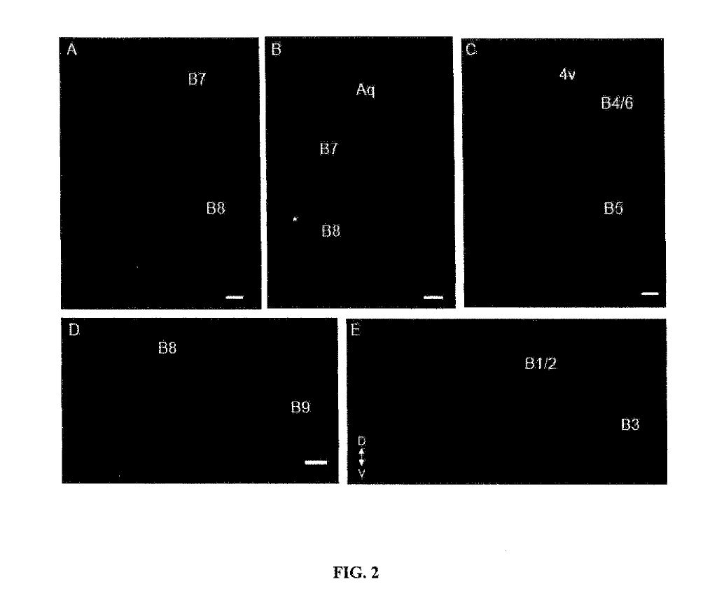

A MluI site located approximately 40 kb upstream of the Pet-1 exonic sequences was used as a restriction site for extending the transgene control. Because of its large size the 5′ 40 kb transgene, 540Z, was constructed in the pBACe3.6 vector and therefore it is propagated in E. coli as a BAC; hence 540Z is referred to as a mini-BAC transgene. Similar to the results obtained with 52Z, expression of 540Z was detected in numerous adult mid-hindbrain locations that correspond to the serotonin B nuclei (FIG. 2). Furthermore, expression of 540Z was detected in scattered cells lateral to the midline (FIG. 2), where individual reticular serotonin neurons have been previously identified.

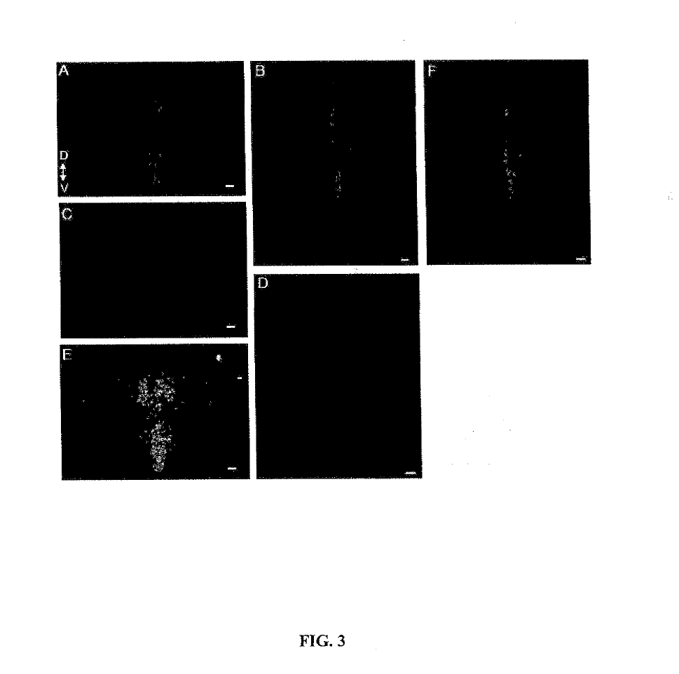

As serotonin neurons are intermingled with large numbers of non-serotonin neurons and glia in each of the B nuclei, an investigation was undertaken to determine whether 540Z expression was restricted to serotonin neurons. Mid-hindbrain sections of adult brains co-immunostained for β-galactosidase and tryptophan hydroxylase (TPH) indicated that all serotonin neurons in the various B nuclei appeared to express LacZ and all LacZ positive cells were also TPH positive (FIGS. 3E and 3F).

Also tested were lines carrying a transgene reporter, r540Z, in which orientation of the 5′ 40 kb genomic fragment was reversed relative to the b-globin promoter. The r540Z transgene was similarly expressed in all B nuclei, which suggests that one or more transcriptional elements functioning as enhancers in the 5′ 40 kb fragment are required for transgene expression in serotonin neurons, but the orientation of these elements in regards to the transcribed gene is reversible.

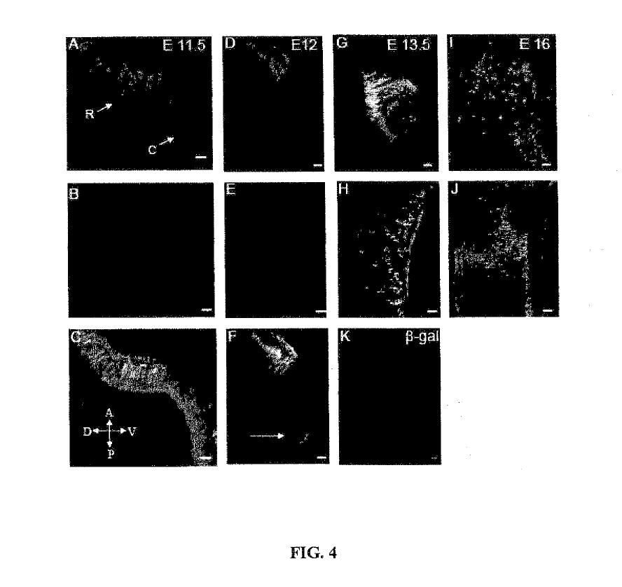

To determine whether or not 540Z properly recapitulated the temporal expression of the endogenous Pet-1 gene, LacZ expression was investigated in the embryonic hindbrain at the onset of serotonin expression. Serotonin-positive neurons first appear in rhombomeres r1-3, the rostral domain, at E11.5 in the mouse and one day later, in rhombomeres r5-8, the caudal domain. In previous studies, it has been shown that Pet-1 expression precedes that of serotonin in the rostral and caudal hindbrain. As shown in FIG. 4A-4C, 540Z expression was detectable in the rostral hindbrain at E11.5. A few cells located in the mantle layer co-expressed serotonin and LacZ at this time but most LacZ-positive cells were serotonin-negative and those positioned closer to the ventricular zone did not yet express the transmitter. A half a day later, (FIG. 4D-F) greater numbers of doubly labeled cells could be seen in the mantle layer of the rostral hindbrain. As expected, few LacZ positive cells and no serotonin-positive cells were seen in the caudal domain at E12.0 (FIG. 4F, arrow). At E13.5 (FIG. 4G, H), extensive co-expression of 540Z and serotonin was observed in both the rostral and caudal hindbrain and by E16 (FIG. 4I,J) a complete concordance between serotonin and LacZ expression was evident. Thus, the temporal expression pattern of 540Z parallels that of the endogenous Pet-1 gene. The specificity of 540Z expression was also evident at E13.5 (FIG. 4K), where LacZ was detected exclusively in the developing hindbrain but not anteriorly, posteriorly or in non-neural tissues. Similar results were obtained for r540Z lines.

Reproducibility and precision of transgene expression. The ability to direct transgene expression to developing and adult serotonin neurons suggested that the 5′ 40 kb genomic region might be useful as a molecular genetic tool for accessing this neuronal cell type. However, the feasibility of this approach also depends on the reproducibility of serotonin neuron transgene expression from line to line and the level of expression in non-serotonergic neuronal and non-neuronal cellular populations. Of the 10 540Z and 6 r540Z lines investigated, all 16 showed appropriate expression in adult serotonin neurons (Table 1). Moreover, of the 15 540Z and r540Z examined in the embryonic hindbrain all 15 showed correct developmental expression with respect to the endogenous Pet-1 gene (Table 1). LacZ expression was also investigated in sections from the olfactory bulbs to the spinal cord. Significantly, no LacZ expression was detectable in other regions of the brain in 7 of the 10 540Z lines (Table 1); the remaining three lines showed LacZ expression in small numbers of cells scattered in the midbrain. The extent of peripheral 540Z expression was then investigated. The endogenous Pet-1 gene is expressed in adrenal chromaffin cells and retinal ganglion cells but none of the 540Z lines investigated showed expression in these cell types (Table 1). However, transgene expression was detected in a very small subset of intestinal enterocytes beginning at E10 and extending into adulthood (Table 1). In addition, LacZ expression was consistently detected in a small subset of cells in the pancreas. Therefore, a small subset of enterocytes and pancreatic cells may be a previously unrecognized site of endogenous Pet-1 expression. A general survey of peripheral neural and non-neural adult and embryonic tissues such as heart, lung liver kidney and spleen revealed no ectopic expression in the same seven 540Z lines that lacked ectopic expression in the brain, while the three remaining 540Z lines showed ectopic LacZ expression in small numbers of scattered cells in discrete locations such as neural crest, lung and kidney. Of the six r540Z three showed no ectopic expression while the remaining three showed a low level of ectopic expression similar to that seen for the 3 ectopic 540Z lines. Similar levels of ectopic expression were seen at embryonic stages. Thus the invention shows that in the brain the 5′ 40 kb fragment is able to direct highly reproducible transgene expression exclusively to serotonin neurons. Moreover, the 5′ 40 kb fragment directed transgene expression to a subset of peripheral Pet-1 positive cell types and little or no ectopic transgene expression was detected in the brain or periphery.

### Example 2

This example illustrates the use of the ePet-Cre recombinase serotonin expressing mice to selectively delete genes from serotonin neurons. Both the conditional R26R Rosa LacZ allele and a conditionally targeted kinase, Erk2, were deleted from serotonin neurons.

ePet miniBAC method and applications. The reproducibility and precision of transgene expression in the brain suggested a novel approach for reliable high throughput expression of genes in serotonin neurons. This approach can be referred to as the ePet miniBAC method as transgenes are built in the pBACe3.6 BAC vector and transgene expression in serotonergic neurons appears to result from Pet-1 enhancer (e) sequences contained in fragment 540. One feature of this method is the ability to rapidly construct miniBAC transgenes using standard restriction endonuclease/T4 DNA ligase plasmid manipulations in the pBACe3.6 vector. In FIG. 1B, is presented a two step procedure where a gene or cDNA can be subcloned into a shuttle vector in order to supply the b-globin minimal promoter sequences followed by excision and subcloning of promoter-gene sequences downstream of the 5′ 40 kb genomic fragment in the pBACe3.6 vector.

To demonstrate the utility and convenience of this technique, two kinds of mouse lines were generated. For the first line, the LacZ reporter gene was replaced with a cDNA encoding Cre recombinase to construct an ePet-Cre mini-BAC transgene. For the second line, LacZ was similarly replaced with EYFP to construct ePet-EYFP. After removal of pBACe vector sequences the transgenes were injected into pronuclei to produce several independent lines.

LacZ was removed from BGZA and replaced with 5′ Nail, AgeI, MluI, AflII, HindIII-3′ polylinker (FIG. 2). A Cre Recombinase cDNA and SV40 polyadenylation sequence or EYFP coding sequences from pEYFP containing a polyadenylation signal (Clontech) were subcloned downstream of the beta-globin minimal promoter using the AgeI and AflII sites. The beta-globin/Cre recombinase/polyA and beta-globin/EYFP/polyA regions were then released from the BGZA by EagI digestion and subcloned downstream of the 5′ 40 kb genomic fragment present in the modified pBACe3.6 vector. The orientation of the genomic fragment relative to Cre recombinase gene was determined using the primers 5′-CAT TTG CTT CTA GCC TGC AG-3′ and 5′-ATG TTT AGC TGG CCC AAA TG-3′ generating a PCR of product size 375 bp. The orientation of the genomic fragment relative to EYFP sequences was identified using the primers used to assess the orientation or 540Z. The two different primer pairs used for the Cre and EYFP transgenes allowed for simultaneous identification.

Founders were used for all analyses of 2PZ and 323Z transgenes and F1 offspring for 540Z, r540Z founders. ePet-Cre F1 offspring were crossed to ROSA R26R mice. The PCR primers used to detect the ROSA LacZ gene were 5′-AAA GTC GCT CTG AGTT GTT AT-3′, 5′-GCG AAG AGT TTG TCC TCA ACC-3′ and 5′-GGA GCG GGA GAA ATG GAT ATG-3′. The cycling parameters were 94° C. for 240s, 40 cycles of 94° C. for 30s, 60° C. for 60s, 72° C. for 60s. The wild type product size was 575 bp; the R26R signal produced a 325 bp product.

Mice 4-6 weeks old were transcardially perfused with 4% paraformaldehyde, 1×PBS pH 7.4 followed by 3 hr post-fixation in 4% paraformaldehyde, PBS pH 7.4 at 4° C. Tissue was then placed in 30% sucrose, 1×PBS pH 7.4 overnight at 4° C. Tissue sections, 20 μm, were obtained using either a freezing microtome or cryostat. Embryos were fixed by immersion in 4% paraformaldehyde, 1×PBS, pH 7.4 for 30 min at 4° C., then placeded in 30% sucrose, 1×PBS pH 7.4, overnight. Fixed tissue was mounted in OCT medium and stored frozen at −80° C. Sections 10 μm were obtained using a cryostat. X-gal staining: 5-bromo-4-chloro-3-indolyl-β-D-galactopyranoside was dissolved in 5 mM K3Fe(CN)6, K4Fe(CN)6, 5 mM MgCl2, 1×PBS pH 7.4 at 1 mg/ml. Fixed whole embryos or tissue sections were incubated in X-gal solution overnight at 37° C. Tissue sections were counterstained with 1% neutral red, then dehydrated in ethanol and mounted. Embryos were rinsed in 1×PBS, pH 7.4 and analyzed for LacZ expression.

The following anti-sera were used. Rabbit polyclonal anti-5-HT (Diasorin 1:10000), mouse monoclonal anti-tryptophan hydroxylase, mouse monoclonal anti-cre recombinase, rabbit polyclonal anti β-galactosidase, goat polyclonal β-galactosidase. Secondary antisera were used at the following dilutions. FITC 1:200, Texas Red 1:400, CY3 1:800. Fixed tissue sections were dried after mounting onto superfrost plus slides for 1 hr. Slides were washed in PBST (1×PBS, pH 7.4, 0.1% Triton X-100) for 15 min and subsequently blocked for 2 hr in PBST, 2% normal serum. Slides were then incubated in PBST, 2% normal serum and, 1° anti-serum overnight at 4° C. Slides were washed 4× for 5 min at room temperature in PBST followed by incubation with 2° anti-sera for 2 hr in the dark. Slides were washed for a final time in PBST 5 min at room temperature and mounted using Prolong anti fade reagent. Fluorescent images were collected on an Olympus BX51 microscope with a Spot R/T colour digital camera. Post processing of certain images was performed using Adobe Photoshop.

Transgenes were isolated from the pBACe3.6 backbone by AscI digest and from the BGZA vector with KpnI and SacII for 2PZ or AscI/Kpn I for 52Z. Constructs were purified for pronuclear injection according to Monani et al. Pronuclear injections into hybrid c57B6/SJL F1 zygotes were performed by the Case Transgenic and Targeting Facility. Founders for each construct were genotyped by PCR. 540Z founders were identified using the same primer pairs and cycling conditions that were used to determine the 40 kb insert orientation. The PCR primers used to identify 2PZ founders were 5′-GCT ACG CCT ACC GCT TTG AC-3′ and 5′-CTG CAG GCT AGA AGC AAA TG-3′, product size 189 bp. Primers used for identification of 323Z founders were 5′-ATC TGG GCC GAG GAT TTC-3′ and 5′-CTG CAG GCT AGA AGC AAA TG-3′, product size 451 bp. Cycling parameters were the same as used for 540Z. The ePet-Cre founders were identified using the previously described primers and cycling conditions that were employed to determine orientation, while the ePet-EYFP founders were identified using the PCR primers 5′-TAT ATC ATG GCC GAC AAG CA-3′ and 5′-GAA CTC CAG CAG GAC CAT GT-3′ product size=213 bp. Cycling conditions were as follows: 94° for 60s, 38 cycles of 94° for 30s, 56° for 30s, 72° for 30s followed by 1 cycle of 72° for 300s. The 2PZ founders were also detected using the same primer pair and cycling conditions used for 540Z. r540Z founders were identified using the same PCR primers and cycling conditions used to determine fragment orientation.

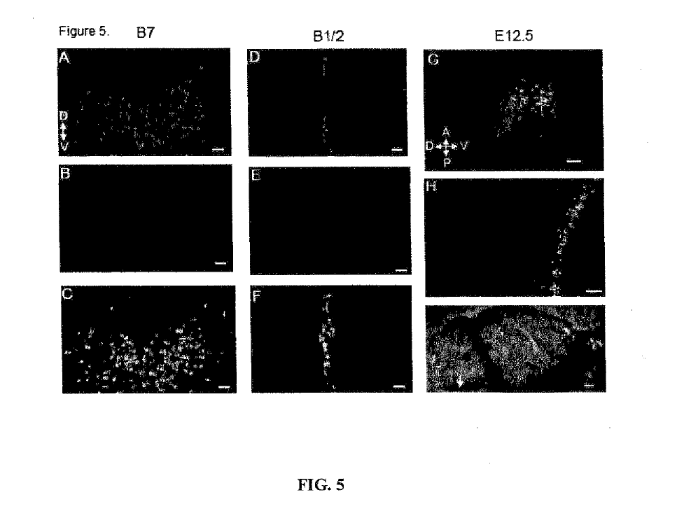

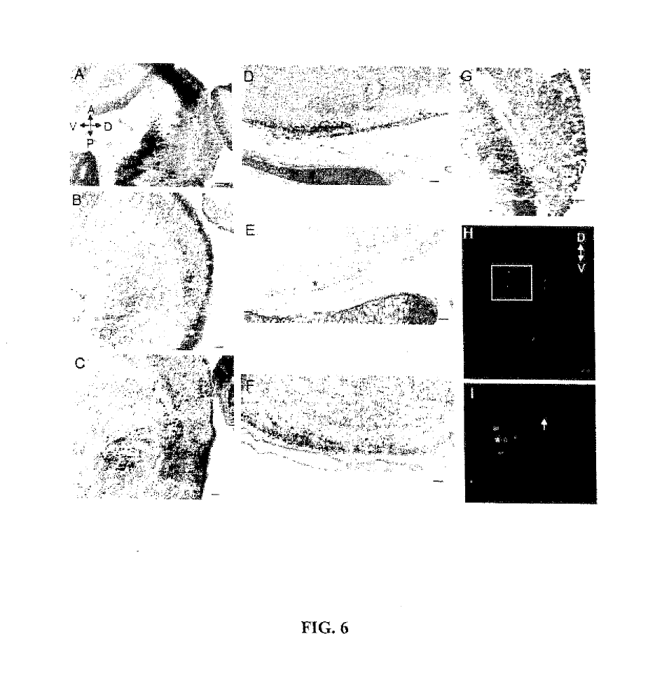

To determine whether ePet-Cre is able to recombine genomic sequences specifically in serotonin neurons these lines were crossed to the ROSAR26R indicator strain. Progeny carrying both alleles expressed LacZ in TPH+ neurons of all B nuclei (FIG. 5A-F). Moreover, virtually all TPH+ cells were evidently LacZ+. Expression in non-serotonin neuron cell types of the adult brain was undetectable (FIG. 5I) and with the exception of very small numbers of intestinal enterocytes and pancreatic cells, no LacZ expression was detected outside the brain in general surveys of adult and embryonic tissues such as heart, lung, liver, kidney, adrenal, gland bone marrow and superior cervical ganglion. Cre expression was detected at the onset of serotonin neuron production in both the rostral and caudal hindbrain domains (FIG. 5 G, H) with ROSA LacZ expression being observed at E13 (FIG. 6 G). Thus, the ePet-Cre line can be used for gene targeting during early serotonin neuron differentiation.

A second line, ePet-Cre (1), was also characterized in which recombination is delayed and occurs in a subset of serotonin neurons. In this line, recombination is not detectable until E16, by which time serotonin neuron differentiation has ceased (FIG. 6 B,E). Maximal levels of recombination generated with ePet-Cre (1) was reached at E18 in both the rostral and caudal domains (FIG. 6 C,F). However, recombination was detectable in about 50-70% of serotonin neurons in each of the B nuclei (FIG. 6 H,I). Thus, the ePet-Cre (1) line will be useful for investigation of gene function after serotonin neuron generation is complete.

### Example 3

This example illustrates that mice containing the EYFP protein expressed in serotonin neurons from ePet-EYFP 40 kb fragment, when coupled to a fluorescent protein-encoding gene, such as EYFP can be used to facilitate the identification of serotonin neurons for electrophysiological analysis.

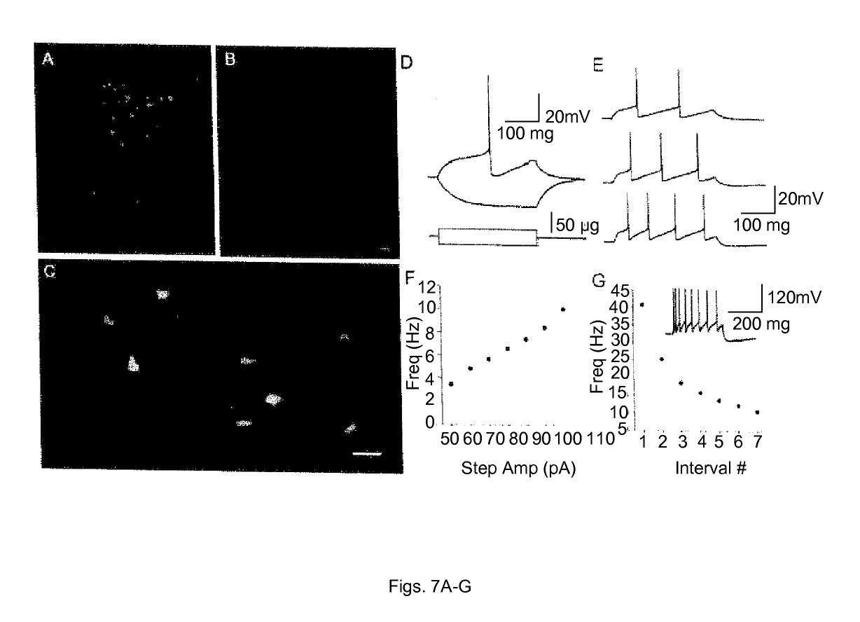

A line in which serotonin neurons are genetically marked by expression of a fluorescent protein would have a number of applications including preparation of serotonin neuron specific cDNA libraries and probes for microarry based studies. Such a line would also obviate immunostaining of tissue sections during neuroanatomical and electrophysiological studies of serotonin neurons. As described above, an ePet-EYFP line was identified (FIG. 7A,B) in which the marker co-localized precisely with the serotonin neuron marker TPH (FIG. 7C). To demonstrate the power of this line a subset of non-raphe serotonin neurons was characterized in the midbrain B9 nucleus. Serotonin neurons in this nucleus are intermingled with large numbers of non-serotonerigic neurons, which has made their characterization with conventional methods very difficult. Therefore their electrophysiological properties are largely unknown

Electrophysiology Coronal slices (250-300 μm thick) through the median Raphe nuclei were prepared from P14-18 mice using a modified Leica VT1000S vibrotome. Slices were incubated at 30° C. for 25 min then maintained at room temperature until needed. Whole-cell patch-clamp recordings were made in B9 neurons visualized under infrared-differential interference contrast and epifluorescence optics (Zeiss Axioskop 1 FS) using an Axopatch 1D amplifier (Axon Instruments). During recordings, slices were superfused with artificial cerebrospinal fluid (ACSF) that contained (in mM): NaCl 124, KCl 3, NaH2PO4 1.23, NaHCO3 26, dextrose 10, CaCl2 2.5, and MgSO4 1.2, equilibrated with 95% O2/5% CO2 and warmed to 30° C. (flow rate, 1-2 ml/min). Patch electrodes (3-5 MΩ resistance) contained: 140 mM K-methylsulfate, 4 mM NaCl, 10 mM HEPES, 0.2 mM. EGTA, 4 mM MgATP, 0.3 mM Na3GTP, and 10 mM phosphocreatine. Voltage records were low-pass filtered at 2 kHz and then digitized at 5 kHz using a 16-bit A/D converter (ITC-18, Instrutech) using custom software. Input resistance was calculated from the response to small amplitude hyperpolarizing steps. Membrane potentials indicated are not corrected for the liquid junction potential. Data are presented as mean±SEM.

Whole-cell patch clamp recording were performed from identified EYFP positive neurons with combined epifluorescent and IR/DIC visualization in acute mouse brainstem slices. Serotonin neurons located in the midbrain B9 area generated a characteristic electrophysiological phenotype, observed in five of six YFP positive neurons. This phenotype included high input resistance (951±100 Mohms; N=5) and a long-lasting and large (23.3±1.6 mV) spike after hyperpolarization (FIG. 7D). EYFP-positive neurons displayed repetitive firing with instantaneous firing rates that varied linearly with current step amplitude (FIG. 7E-F). Firing rates adapted rapidly during step responses (FIG. 7G). A sixth EYFP positive neuron in B9 generated intrinsic responses that were distinct from this phenotype, including lower input resistance (380 Mohms), a multiphasic spike after hyperpolarization and prominent low-threshold regenerative responses, suggesting the presence of electrophysiologically-distinct subtypes of serotonin neurons in B9.

Although the present invention has been described in considerable detail with reference to certain embodiments thereof, other versions are possible, Therefore the spirit and scope of the appended claims should not be limited to the description and the versions contain within this specification.

1-49. (canceled)

50. A composition for expressing at least one gene that is exogenous to a serotonin neuron in the serotonin neuron, said composition comprising:
an isolated nucleotide sequence comprising at least one of an exogenous mammalian Pet-1 or FEV enhancer this is exogenous to the serotonin neuron coupled to the least one exogenous gene and at least one carrier;
wherein said at least one of an exogenous mammalian Pet-1 or FEV enhancer enhances expression of the at least one exogenous gene in serotonin neurons and intestinal enterocytes with substantially no ectopic expression.

51. The composition of claim 50, wherein the at least one carrier is selected from plasmids, Bacterial Artificial Chromosomes (BACs), and miniBACs.

52. The composition of claim 50, wherein the at least one exogenous mammalian Pet-1 or FEV enhancer comprises a murine Pet-1 enhancer.

53. The composition of claim 50, wherein the at least one exogenous mammalian Pet-1 or FEV enhancer comprises a human FEV enhancer.

54. The composition of claim 50, wherein said at least one of an exogenous mammalian Pet-1 or FEV enhancer comprises the intergenic DNA fragment between the transcriptional start site of a murine Pet-1 gene and the transcriptional termination site of the next gene 5′ to Pet-1.

55. The composition of claim 50, wherein said at least one of an exogenous mammalian Pet-1 or FEV enhancer comprises up to the entire intergenic genomic DNA fragment between 5′ of the transcription start site of a serotonin specific transcription factor and the 3′ transcriptional termination site of the proceeding gene.

56. The composition of claim 50, wherein said at least one of an exogenous mammalian Pet-1 or FEV enhancer comprises the intergenic DNA fragment between the transcriptional start site of a human FEV gene and the transcriptional termination site of the next gene 5′ of FEV.

57. The composition of claim 50, wherein said at least one of an exogenous mammalian Pet-1 or FEV enhancer comprises a 40 kb DNA fragment 5′ of the transcriptional start site of a murine Pet-1 gene.

58. The composition of claim 50, wherein said at least one of an exogenous mammalian Pet-1 or FEV enhancer comprises Seq. ID NO. 10.

59. The composition of claim 50, wherein said at least one of an exogenous mammalian Pet-1 or FEV enhancer comprises a 40 kb DNA fragment 5′ of the transcriptional start site of a human FEV gene.

60. The composition of claim 50, wherein said at least one of an exogenous mammalian Pet-1 or FEV enhancer comprises Seq. ID No. 11.

61. The composition of claim 50, wherein said at least one of an exogenous mammalian Pet-1 or FEV enhancer comprises a 2 kb DNA fragment 5′ of the transcriptional start site of a murine Pet-1 gene.

62. The composition of claim 50, wherein said at least one of an exogenous mammalian Pet-1 or FEV enhancer comprises a 2 kb DNA fragment 5′ of the transcriptional start site of a human FEV gene.

63. The composition of claim 50, further comprising a pharmaceutically acceptable carrier.

64. The composition of claim 50, wherein said at least one exogenous gene encodes a protein selected from the group consisting of a bacterial enzyme, a recombinase, a reporter protein, and combinations thereof.

65. The composition of claim 64, wherein said reporter protein is selected from the group consisting of Green Fluorescent Protein (GFP), Enhanced Yellow Fluorescent Protein (EYFP), LacZ, Fire Fly Luciferase and combinations thereof.

66. The composition of claim 64, wherein said recombinase is selected from the group consisting of site-specific recombinases, tyrosine recombinases, serine recombinases, Lambda-Int recombinase, Cre recombinase, Flp recombinase, HP1 recombinase, XerD recombinase, and combinations thereof.

67. The composition of claim 50, wherein said at least one exogenous gene is a therapeutic gene.

68. The composition of claim 50, wherein said at least one exogenous gene encodes a light-sensitive protein.

69. The composition of claim 58, wherein said light sensitive protein is a light-sensitive ion channel.


SEQUENCE LISTING (11 sequences)

SEQ ID NO: 1 - DNA, 1752 bp, Rattus norvegicus
tttgttttaacaaacatgtttattagaaaagtaaaaatattgcataggtcttagtacttgaacatcaagtgtattcatgaaccgtgagtatcttcatgtaaacagttctagatggaagacccaggtggcgctcctctgggggagagggttccagccccccacccccctcagccccatcccctcacagctcactcctccagtacaccggcaccgggatgggctgggatgcagctccaggaccccctccctcctcaccacaccctggctgcccgctcccgccaggccttgccggacccggcggcgtctactcttccctgtcacccacagtcaccacgggcgggtatcggcaccccaagcgcaaagctgacgtgcccccccgtgcggtcccccccatctcccaccgcccagtccccggcagcgatgagacagagcggcacctcccagcccctgctgatcaacatgtacctaccagatcccgtcggagatggtctttttaaggaagggaagagcccgagctgggggccgctgagccctgcggtacagaaaggcagcgggcagatccagttgtggcagtttctactggagctgctggcagaccgcgcgaacgccggctgcatcgcgtgggagggcggccacggcgagttcaagctcaccgaccccgacgaggtggcgcgacgctggggcgagcgcaagagcaagcccaatatgaactacgacaagctaagtcgagcactgcgctactactacgacaaaaacatcatgagcaaggtgcacggcaagcgctacgcctaccgctttgacttccagggcctggcacaggcttgccagccaccacccgcgcacgcccacgccgccgctgccgccgccgcagcggcagccgccgcccaggatggcgcactttacaagctcccggctggtctggctccactgcccttccccggcctctccaaactcaaccttatggcagcctcggccggcgtggcgcccgctggcttctcttactggcctggtcccaacgccaccgccgctgccgccgccaccgctgcgctctacccaaccccgggcttgcagccccctcccgggccctttggcgcggtggccgccgcttcgcacttggggggtcattatcactagacgggacggccgggtgcagtggggcctctcccacacagccagtgaccaatcccatcctcatcctgggaggagccccgaagatttccccgacgttcctttaccacagatttcgttgcagcagccgctcccagcccagggaagaaaggatgggaagcctctgaggtcttccttgaatacgaggcttccaggctcccattatcatcaccccaggaagggtgcatgtgctcccactttaatttttctcttccaagtctccagattctggaactcccgtcttttttttctcttctcacctggagcccctgccttcctctttatgacccctagttttctgttttgttttttttttttcctctctctctcctcattttttttctctcccacgacctactccaaacggtagtacctcggtagtacctcgaggcttctcacactccccttttcgggatatgagaagcatcaaaaacatctctgctgttgtccatccctatcccaacactctggcttcgctcccttccataccacactctggcccaaggaccctcgtctgtatatattcctttcagccccattaaagatccaagcttcaaaaaaaaaaaaaaaaaaaaaaaaaaaaaa

SEQ ID NO: 2 - protein, 45639 aa, Rattus norvegicus
TCCCCCAAGCCCAGAAGGCTTTGCCTTTTGTCTTCCATGATGTTACAGGCTAAATTTTCCCTGGGGCCACCTGCCTTCTTCGAGGGGCTGTTCAGCTGGGGCCACAGAGGCCCTGGTGGGTTCCCCCAAGACTCCCCCATCTCTGCCCCAGCCAAGTGCGCCCAGCTGCTGGTGAGCATAAAGCACAAGTGCCCGGAGGACCAGGATGCCGAGGGCTTCCAGAAGCTGTTGTACTTTGGCTCTGTTGCTGTGGGCTGCACCTCGGAGAGGCAGATCAGGCTACACAACCCGTCGGCGGTAGGCACCAGGCAGGAGGGGCGGCAGGACTCACGCCCCCAGGATTGGAATTTCCTAAAATCACCGCCTTGCAGAATGCCCCCCCTTTCTCTCCATGCCTTTGGCCCCAAGCCTCATGTAACCCATGTCAGCTGTCAACAGGTGTGTCCTTCTCCTCAGCTGGGGGCTGGGCTGGGGTATACCCTGGCTCCCCAGAGCCACGAGCAGCCCTAAGACTGTCCTGACCCCAGGACCACAGTTAGCTGTGATGGTACCCTGGGGAGAATTAAAAGGTGCCCCCTTTGGGCAGGCCCAGCGCAGGGCACATGACTCGGTCCCTTCTCTCCTCCTGGTGGAGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGTGCTCTGCTCTGTGACCTGACCCTTTGTCCTTCTGCTTGGCCACACAGGTAAATGCCCCCTTCAGGATTGAAATTTCCCCGGATGAACTGGCCGAAGACCAGGCCTTCTCATGCCCCACGGCCCATGGCATCGTGCTTCCGGGAGAGAAGAAATGTGTGTCGGTGTTCTTCCACCCCAAGACTCTGGACACCAGAACTGTGGACTACTGCTCCATCATGCCTTCTGGCTGTGCCTCCAAGACCCTGCTTAAAGTCGTTGGTTTCTGTAGAGGTACCGGTGACCCCAGGGGCCTCCCCTGGGGACAGGAGCAGCCCCTGCTGAGGATTCTGAAGAGCTCCTTCCCTGCTCCTAGGAGGCGGGGCCACCTTGCCTCAGCCTGGGTGTCTGCTGCCCCCTGGTGGCCCTGTGGGGAATCATCCGTGACTACCTGGAGATCCCAGTCCCACTGGCATCCAGTGATGCTGGAGTTCTGCTCAGGCTGCCAGGGCTCTGGGCCTGTAGCCTGGCCCTGAGAGTACCACCTCCCTTCAGTGGAACTTTGTCTAAGATATCCTTGGGGAAGCTGATACCCCATCTCCTGTGTCCAGGCCCTGCTGTGTCCCTGCAGCACTACTGTGTCAACTTCAGCTGGGTCAACCTTGGGGAGCGCTCCGAGCAGCCCCTGTGGATTGAGAACCAATCGGACTGCACGGCCCACTTCCAGTTTGCCATCGACTGCTTGGAGAGTGTCTTTACCATCAGGCCTGCCTTTGGGACGCTGGTGGGCAAGGCCCGTATGACCCTGCACTGTGCCTTCCAGCCCACTCACCCCATCATCTGCTTTCGGCGTGTGGCCTGTCTCATCCACCACCAGGTGAGTCGTGGGGAGGGCATGAGGCTGAGGGAGGAGGGGCTCCCCTGGGGCCCACTGATGATGACAAGGCTTGGTGGGCACTTCCCTGTATTCTGGAACTTCTAGGGCTGTCTGCCTGGTGGTCTCCCACTCCTGGGCCCCAGCCCTGGCTTTTGGAGCCAGTGTGCAGCCTGGGCACAGGGCAGATCACTCTGTGCCAGTCTTGCCCTGCTACCAGGCCAGGCGCTTTGCTGCTGCTGTGCTCATGGGCTGGGAGAGGGAGGCTTGGACTCGCAGCCTCCCACCCTCCCGCTGAGCCCTGCAGGCTTTGCCTTTTCTTTGCCTTCCATGACTTCACAGACTAATTCTTCCCTGGAGCCACCCCACATAGCATGCATAGGGCAGAGAGCTTTCAAGTTAGACCCCAACCCTAGGTCCCTGGGCATGATCAGACACANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGAAATGGAGGAAAGAACAGAAGCCCAGAGGGAGGCCTGATGAGGCAGCCCCCCATATTCTCCTCCCTTGGGAGGGCCTGAATTCTAGTTTGGCAGGCCTCTGGGCTGGCAGTAGGGTGGGTGGGAGAATGCCCAGGGGCCATGGCAGAGAGGAAGGGTGGGTAATGGCTGAGTGTCCTAAGGACCGGGGTGCTCTCTCCTGACCTTCCCTGGACTCCTGCTGTGCCCTGGCTGAGCCAGACCACTGCTGAGTGCTTGCTCACTGGCCACCTGGAGCTTCTGCCCCACTGCCTCTGTTATCCTGTCTGCCCAGCTTCTGTCCTCAGTTCAACCCCCAGCACCCCCCTTGCCCTACCACCCCTCCCATGCCCCACCACACACAGACAAATGTCACAGGACCCACTGTTCCTGGACCTGATGGGGACCTGCCACTCGGACAGCACCAAGCCAGCCATCCTGAAGCCTCAGCACCTCACCTGGTACCGCACACACCTGGCCCGGGGCCTGACGCTCTACCCCCCTGACATCCTGGATGCCATGCTGAAGGAGAAGAAGCTGGCACAGGACCAGAACGGGGCTCTCATGATTCCCATCCAGGTACAGTGCCCTCCCCAGCTGCATATCCCTTCTCTAGTGATTCTTGGGCAATCCCATGCCCCCAGGGTCCCATGGGCACCATTAGTAGTCAGGGCGACTGGTGTAGCCTTTCTAGACAGGAGTGTACCCAGGAGGGGCAACAGCAAGAGGAATGGCAGTTGAGCCCTATCCACCTGTCTTTCCCCATGCAGGATCTGGAGGACATGCCGGCCCCGCAGTACCCTTATATCCCCCCCATGACCGAGTTCTTCTTCGACGGCACCAGCGACATAACCATCTTCCCCCCGCCCATCAGTGTAGAGCCTGTCGAGGTAGACTTCGGTGCCTGCCCAGGGCCTGAGGCCCCCAACCCTGTACCCCTGTGCCTGATGAACCACACCAAGGGCAAGATCATGGTGGTCTGGACGCGAAGGTCTGACTGCCCCTTCTGGGTGACTCCAGAGAGCTGCGACGTGCCCCCACTCAAGTCCATGGCCATGCGCCTGCACTTCCAGCCGCCTCACCCCAACTGCCTTTACACGGTGGAGCTCGAAGCCTTCGCCATCTATAAGGTGTGTGCACGCAATGAGAGGGAGGAATGCGGGGTCTCTGCTAGGAGCCTGAGTGGCTTGGTGGGGTGGCAGGAAGTGACCGAGGGCAGCTTCAGGCTCCATCCTCTGCGTGCCAGGCTTTCTCTTGGCTGGACAGTGACCCCTATGAGTTTGTCTCCTCCAAAGCTCCTGGCCTAGCTCGGCCCATCTGATTTTCTCATTCTTATGTAAGTCTCCCCTCCCCTCCAAGGGAGAACTCAGCTGAGATCAAGCTGTTTGGGAAAACTGGGTGCACAGGGAGATACTCCCTGGGGCTCCTGGCTAGGAGGCCTCCTAGCTTCTCTACTAGTCCTTGAATTAAGAAGTGGTCACTCTAAAGGAGCTTTGAGCGGGCAGGAAGCTGGGCCTAGAGACAAAGTCAGCAGCACCAGATAATTGTGATGGAAAGGGCTTCTGACTCAGCTTCCCTGGGTCGGGAACTCCGAGTGCCGGCTGTCCCCAGCCCTGCTGTTCTGGCCCCAGATGCGTGGTGCCCCCTCCTCATCCGTTAGTCCTGCCCATCCCTTCCTTCTTGACTCTGCCCACCCCACTGCCCTTGCCCAGAGGCCAAGGTCTTGGGGCCCAGAGAAAAGTAGGGCTGTGCGGTCAAGATCAGGNNNNNNNNNNNNNNNNNNNNNNNNNNNNNNNNNNNNNNNNNNNNNNNNNNNNNNNNNNNNNNNNNNNNNNNNNNNNNNCCACGTCCGATCTTTTGAGGAGGCTTTCCAAAGTGGAGGCTTTGGTTGCCCCCGTCCTAATGCTCTGGCAGTGGGGTGATGTTGAGGTTTGTAGGAATAAGGTTGTAGATGCCTGGCTCTGCTGAGGTTCAGCCTGTCAGATATTTAGGTTACAGGCTCTAGACNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTCTCTAGGCTCATTCTTACAGAATGAATGGTGCTTTCACATCCCAGCCCCACCCTCCTCCCGGGTTGTGAGGACTAAAGAGAGCATTGCTATATGGATGCAGGGCCTAGCTCCCCGCAGGAGGGGCTGGGTTGGGATTCTTGTTCTCTGCACTGTCTTCCCAGGGAGGCCCTCCTGTCTGTGTCCAATGTCAGACTCCTGCAGCACAAGGGCAAGGAGGGGCAAAATGGGGTGCCCCACAGGGCAGACACAGGACTCTGAGCTTTTCCGTGGGTCTGGCCCCTCTGCAGGTCCTGCAGAGCTACAGTAATATTGAGGAGGACTGCACCATGTGCCCATCCTGGTGCCTGACGGTGCGGGCACGAGGCCACAGCTATTTCGCTGGCTTTGAGCACCACATCCCCCAGTATTCCCTAGATGTCCCCAAGGTAAGCGTGGCCCCAACAGCAGTGGGAGGGAGACAGGGGAGCCCTAGCTTCTGCCTTGGCCTGCAGATCCCTCTCCCATTCTCTGATGGCCTCAGAAACACATCTTTGCACCCTCCCTGGCTGTCCCCACGTCCGCTGTGCCAATTCTGGGTATCTGGGCCGGCTGAGGACTTGCTCCTGGGCCCCGACACTGGGACTGGGGAGAGTCTGGATCCTGGACAGCTGGCCCCTTGCTGCTCCATTGGAAATACTCACTGGCTCCTATGACTCTCAGGTTCAAAACCCTCCAGCTCCTGCATTTGTTTGTGTCCTGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGCCACCCCAAGGCCCTTAGAAGCCCTGCCTGACCTGACAAGCAAGAGGANNNNNNNNNNNNNNNNNNNNNNNNNNNNNNNNNNNNNNNNNNNNNNNNNNNAGTTTCACCTTTGATGTCTCCTCCAGTCTCCATCGCCTGGAGGTTTTGCATTAGACTTCTGGTTGCTGGAAAAGCACCACTTTGGGACAATGTGCTTTGGGTTGATTCTGTTCCCCCTACCACCTACACTTCTGCCCTCCTATCCCAAACATATACCACCCCAAGATTCAGCTTAGAGGCAGCTGCTTCCCAAAGGCCTTCCTGCCTCACAGAGTCCCCCTCGATGTCTCCCTCGGCCCCCTTCCCTTGGCAGTTCCTCCTGGTGGGTTGTGGCCTTTATGCAGCCCAGAAGATCTGGCGTCACTCCCTCACCGGCTAACACACAGGCACTGTCACCCCAGCTTGATGGGGACCTCTGGTCAGGGATTGCAGGTTTTGTGTTTTGCAGCCTGCTCAGCTCTCAGCTGGTGCTGGCCACACCCTGGATACCTGAAGAGTGTTGGGAGCTGAGATTATCTGGGCCAGTTTCCATCAGTTACCTGCATGAGAATCATCTAGAAGGGGTTACTTACNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCGAAGTGGTTACTTACTGCAGCCCCAGAGCTTCTGGACCAGGCTCTCTGAAAATGAGTTCTCCAAAGAGTGATGCAAAGACACCCTAAAATGAAACAACTTCTACTTTATCCTTGAGGAACGGCCTTGGAGGCCTGAGTTTCCCAGGGGCCTGTNNNNNNNNNNNNNNNNNNNNNNNNNNNNNNTTTCTGGAGGTCTCTGGAGAACAAAAGCATTCTTTGGTGTCAGCTCGTGGCCTCCTTCAATTCCCCGTTCCCAGGGCGTTGACTGGGCTGCAGGGCAGCACCTCTGATCTACTTGGAGACAGCTCCTCCCAAGCCCCCATAGGGCCCTGAGTGTGTGTCCCAGGGTGGAGGTCTGAGCGGGGGGCCGCTTTCCTCCTCTCCCACCCCCAGCTATTTCCAGCAGTGTCCTCCGGTGAGCCCACCTACCGCAGCCTGCTCCTGGTCAACAAAGACTGCAAGCTGCTGACCTTCAGCCTGGCCCCCCAGAGAGGCTCAGACGTCATCCTTCGGCCCACTTCGGGCCTTGTGGCACCCGGGGCCCACCAGATCATCCTCATCTGCACCTACCCTGAGGGCAGCTCCTGGAAGCAGCACACTTTCTATCTGCAGTGCAATGCTTCCCCCCAGTATCTCAAGGTAGGAGGCAGAGGGAGGGGACCTGGGTCCTTGGGAATGGGCCATAATCTTGAACCACCCTTTTCAAGGCCATATTCTGGGGTTGGCCCCTAAGATTTCCAGGGGGCCACTTCTCCTCCAGGAAGTAGCATAGTAGGGAAAGAACCTGCTTGACCACTTCTGTGAACAACTCTCCCACCCCTCAGGGCCTTGCCTGTCCCCCTTATGGGGTCTGAGGCCAAGAAAGAGGGTGCAGAGAACCCAGAGGAAGGATCTGGGGGAGGCAAAAGACACCTGCAGGTGGTAGAGGGATATTTGGGTCCAGGCCAGCAATAGACTCTGAGATGGTGGGCCCATGTCTGGCCCCGTGGAATGGTCTATAGATCCTCTCTCAGTCCCTGAGTTCTTTTCAAGAATTATCTCCTGGGCCAGTCACCATTGACTCTGCTTTTGACAGAAACTGCCGGTACACGGGCTGGCAGAGGGCCTTTGACTGCCTGCTTCAAGTGGGGAAAGCTGCCTAGCCATGGCTTCCTGGACATGTTGTGGGGGGCTGGGACTGCAGAGTGAGGTCAGGGCACTGCCCTTCTTCCTCTGGCTCCAGGAGGTGAGCATGTACAGCCGGGAGGAGCCACTGCAGCTGAAGCTGGACACCCACAAAAGCCTCTACTTCAAGCCCACCTGGGTGGGCTGCTCCTCCACCAGCCCCTTCACCTTCCGCAACCCCTCGCGTCTGCCCCTGCAGTTCGAGTGGAGGGTCTCTGAGCAGCATCGAAAGCTGCTGGCTGTCCAGCCCTCCAGGGGGCTAATCCAGCCCAACGAGAGACTTGTGAGTGGCTCCCCTCCTGCCGAGTGACGGCAACCTTCACTTCTGTCATCTCTTATTTGTGCCTATCATAGCCCAGACTTGCCATGTGCAGCCCCCAATTCCATCCACAGTAACTTCCCCCACCTGGCGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTGAGAGGCATGTCACCTCCCCATCCATGGTCCCCAAAGCTGGGAGGTCATTGGGAGGGTGGGTTGCAGCAGCTGGATTTGGGCCCAAATGGCCAGCCACCAGTGAGGGTGAGGAAGGAGACTGTAGTCACAGTGGGGGTGCTGGCTGGGATTCAAAATAGCAGACCCCTGTCCCTAGTCTAGAACCTTACCGACGGGTTGGGGAGGCCAAACTTGGGACAAGAAGCACCAGCACAGGCATCTGTAAGAGCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGTAGCATGACGCCCTGCCATGGGGCCTCGGATGGCCAGGTACCTGTGGAGGGGCTTCCGAAGGCCACTGCAGGGATCATTTCTGTGACCCCAGACGCTGACGTGGACCTTCAGCCCTTTGGAGGAGACCAAGTACCTGTTCCAAGTGGGGATGTGGGTCTGGGAAGCCGGCCTGTCCCCAAATGCCAACCCCGCTGCCACCACCCACTACATGCTCCGGCTGGTGGGCGTTGGGCTCACCAGCAGCCTCTCTGTGAGTGGGAGGGCTCTGGCAGGAGGGGGCTGGGGGAGAGGCCCGGACAGGTGGGAGGGAAGGCCCCAGGGAACCTCCCAAGGTGGGAGGGAAGGCCAAGAGGGCAGAGGATAGGAGCCCTCTGCCCCGGATACCCCAAACTTGCCCTTCCTTGGGGGATGCTCTGAAAATGGGGCTGGGAGAGTGGGGCTGTGGGCCTGGAGGAGCTCCCACTCCCGGCTGGCTGACTGTGGCCTCCAGGCAAAGGAAAAGGAGCTGGCCTTTGGGAATGTGCTGGTGAACAGCAAGCAGTCCAGGTTCCTTGTCCTCCTGAATGACGGCAACTGCACCCTCTATTACCGCCTCTACCTGGAGCAGGGCAGCCCTGAGGCCGTTGACAACCACCCCCTCGGTACTTGGGCCTGAGCTGGGACTGGGGCCAGGGGAGGTGGGAGGAGGTACTGGTTAAGACCGGTTAACCTNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGTGTGGTCCCTCTGACCCAGGCTGGGAGGAGGGGCTCCACTCCCTGTTTCCTGCCTCAGGGCCTGCTGTCCTCCCCTGGTGCCCAGGGAAGGGTATCTACAAAGGAGCAGGAGGAATGGCTGGGCCTCCTCTACCCACTGACCCTCCGGCATCATCCCTGAGCAGCTCTGCAGCTGGACCGAACAGAGGGGAGCATGCCACCCCGGTCCCAGGACACCATCTGCCTGACTGCCTGTCCCAAGCAGCGGTCCCAGTACTCCTGGACCATCACCTACTCTCTCCTTTCCCACAGAGGTACCTAGACTGCCTGGCATAGAGAGTCGGGGCCGGGGAAATGCAGGATGAAGGGGGAGGGCTGTGTGAGGCAGTGGATGCCGAGCGTCAGGCAGAGCTGGCAGGGTGAGTGGGGTGTGCTGAGGTGTGCCTTTGGTGTCTGGCATAAATGCCACACACCAGACATGTCTGTCCAGATGAGTGTCCATATGTGCATGTGCCCAGTGCAAAGAGTTAGGAGCTGTGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGTCCAGGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTGGGGATCATAATGTCACACANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTTAGTTTGAAACCAAATCACGGTACACNNNNNNNNNNNNNNNNNNNNNNNNNNNNNNNNNNNNNNNNNNNNNNNNNNNNNNNNNNNNNNNNNNNNNNNNNNGTTGTTGGAAATGCCCTAGAGCCCGAGAGGGGTCCACTGCTCCTGATGAGGGTCCTGGGGCCAACTGCCCAGGACTCCTGCACCTTTGGCCTGCATGCCCCCTCCAGGGAAGGTGGGAGGCAAGCCCACTTCTGTCTTCCTCACCCCCCTCCCCAGATAACAAGGCTGGGGAGAAGCAGGAGCTGTGCTGCGTCTCCCTGGTGGCCGTGTACCCCTTGCTTTCCATCCTGGATGTCAGCTCCATGGGCAGTGCTGAGGGTATCACCCGGAAGCACCTGTGGCGCCTCTTCTCTCTGGACCTGCTTAACAGTTACTTGGAGCGTGACCCCACCCCCTGTGAGCTCACCTACAAGGTGCCCACCCGGCACAGGTGAGCTGAGCCTGGAGTGGGGGTGGGGGCTGGGGGCAGGGCCTATCTAGGGATGTTCATGGAGCTTTCTGCCCAGACTCCCAGAACAAGGAAGCTGTTTCCCATCTCTTTGAGGGAAGAGCTCCCCAGTTCTCGCCCATCCCTGGAGGGGCCCAGGGAAGGGAAGAATTGGCTTGAGGTCTGAAGAGCCAGCCCACCCTTGCGCCTGAGTCCCAGATTCTCCAGTTCCCACTTGGAGGGAGGGGAGTGCCCAAGCCAGCCATCCCTGGGAGGGCCCTGCCCTGGCCCCATCCCCATCTCTGAACCCCTTTCTCTGTTCCTCACAGCATGAGCCAGATCCCCCCCGTCCTCACCCCTTTAAGGCTTGACTTCAATTTCGGGGCCGCACCATTCAAGGCCCCACCTTCCGTGGTATTCCTGGCCCTGAAGAACAGCGGAGTGGTGTCCCTGGACTGGTATGGCTTGAAGGGGGCCACTGGGGGGCCTGGGAGACAAGTGCCTAGAGGCTGAAGGAGAACAAGAGCTGCACTCTCTTGCCCGTGGTGGCCTGGCTGCTTGCTGTCTGTGAAGAGTGGACCTCACATTGACGGCTGTGGAGGCCAGGGTGGAACTGGGGTTGCCAGCAGGACTCCGGGCCTGTCACCGGCTGAGACACGGTGATAGGCTCAGAAGCGGTGGGACTGCCCTGATGGAGCCTGGGCATAGTGGCCTTAATAACTTNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGCAAGAAGACTGAGTTGCTTCTACATAGTACGCACTGCCTCGTTGCTGGAGCCCGCCTGACGGAGCCCCAGCTTAGTGGAGGCTGGGCCTGAATGGGGGCAAGTTGAGGGTCTCTGGTGGGGAAGGACAGGGACCTGCTGCCGGGGACAGAGCTGCCCCCGAGGGTCCTGGCTGAGGGGCTTTTCTCCCCCAGAGGCTCAGAACTTGTGCCCAGCAAACATCCTGCTTCCATGCATGGACTTTTCCTCCCTCCACCATGTGCCTTTGTTTCCTAGTAAACTCCTTTTGATGACTTCCAACTTTGCTTGGCTAAAGAGAGGTGGAAAAAAAAGTGTCCCCATCTGGGTCAGACTTAGGAGAGGGATCCAGGGGTCTGGCCAGGGCATGGCAGTGAGTGGGGGAGAATGAAAATATGCAGGTGGGGGCTCCTGTGATCAGGAACTCGCTGGCCTGAGCAGAGAGAGCCTTGGGGAATGTGGAGTGAGAGGGGTGGGGTCTGCATCATCAGATGGGCACTAGCAGCCCCTCACTCTCTGACTGTCCCCTTTCTCCTCCATGGTGGAGGAAGATGGGGAAACTGGCTCTTTAGGGGGCTCCTGTCTGAGAAGGGCCAGAACTGTGAGTAACTGAGGGCTTCTAGCTCTGAATGACTGGGAACCTTGGCAGAGCTGCCAGCCTGGTGGGGAAGGCCAGGGCCTGCCTTGAGCTCCAGACCTGCCATTCACTGGGCTCTTTGAGTGGGCCCGGAGCCAGGCCCTACAGGGCTCCAGGGAGAGGGTTGGGGGCAGAAAAAGCGGTGTTGGCCTGTGTCCTGGCATGGACTGCGCGCTGGTGTTTGGGTAGGAGCAACCAGCTCACACTCTCTGTGGAGAGGGTCCAGGCTGCTGGAAACAGAGGCGGGGGAAACCCAGGCAGCATGCCCGGCCAGCCGGGCCCATCCATCAGGAGATCATCTGAGGTGCAGGAAGGCAGTCCGTGCCTCTTCCAGGATTCTAAGCAGATTGACTCTCCTATGCTTTTTTGTTGTTGTATTTCCTTTCAATTTCTTAGAAGGCTATTTTTGAAAATTCTAGTCCGTCTCTTCTGAGCTCATAAAGAACAGACGGAGGGTTTTATTCCTGCCAAGGCCTGTCAGAGAGGGCCAGGAGGCAGCAGCAGGCAGGAACCCCAAGTGGGAGGGGGAGCTCCAGGGGAAGCAGGGAACCATGCAGCTGCCCCACTCCCTCCACGCCTTCTCCCCTTCGCGCCTCTGCTTTACCTGGCCCTGGCCTTCGTAGGGTCTTGCATTTAAGGGGAGATTCTTTTCAGATGTCAAATATGCTGGTTAACATCATTTGTTCATTCATCCAGTCTNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCCTCCTGTTGCGGGGTCATATGTGCTGTGAAGGAGGTGTGTGCAGCATTTCANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCTGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGGAGGGAGACTGGACCAGGGCAGAAGGCAGTGAGGACAAAGGAGAGGCTGAAATATTAAAGGACCCCAAACTAGCAAGGTCTCCAATTTTGCCACTGCTGAATATCCACAATAAAATGACAACAGTGGTTTACTCACTTGTAACAAGTCAGACAACATAGGAGCTATGGGAAAAAAGTAAGAAGCTCCGCAGCCCCCTCTCCAAGTCCAGCTGCTAGGGTGATGAGGATTCGGAATGTGCCTTTCACACTCAGCANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCTATTCTTCATGCTTCAGATGTGAAAAAAGTTAAGACTGGTTCCTTCTGAGGGTTTGGACTTAGGCCTCCCTGACTCCTACTGAGGCTTCCAGGCCCGTGTTGCACGCTTTCTCTCAGGTGTGCGTTGTGTGCTTTCTCTCAGGTGTGTGTTGCGTGCCTTCTTTCAGGTGCGTGTTGCACACTTCCCGTCAGGTGTGTGCTGCGTGCATTCTCCTCAGGTGTGTGTTGCACGCGTTCTCCTTAGGTGTGTGTTGCGCACATTCTCCTCAGGTGTGTGTTGTGCACTTCCTCTCAAGTGTGTGTTGTGTGCATTCTCCTCAGGTGTGTGTTGTGTGCTTTCTCTCAGGTGTGTGTTGCACGCGTTCTCCTCAGGTGTGTGTTGCACGCATTCTCCTCAGGTGTGTGTTGCGTGCATTCTCCTCAGGTGTGTGTTGCACGCTTCCTCTCAGGTGTGTGTTGTGCGCATTCTCCTCGGGTGTGTGTTGTGCATTTTCTCTCAGGTTGCTTTTGTGCGCTTTCTCTCAGGTGTGTGTTGCATGCATTCTCCTCAGGTGTGTGTTGCGTGCTTCCTCTCAGGTGTGTGTTGCATGCATTCTCCTCAGGTGTGTGTTGCGTGCTCTCAGGTATGCGTTGCACACTTCCTCTCAGGTGTTTGTTGCGCGCATTCTCTTCGGGTGTGTGTTGCGCACTTTCAGGTGTGTGTTGCGTGCTTTCTCTCAGGTGTGTGTTGCGTGCTTTCTCTCAGGTGCGCGTTGCGTGCTTTCTCTCAGGTGTGCGTTGTGCGCTTTCTCTCAGGTGTGTGTTGGGCACTTTCTTTTCCTTTTCTGGATGCTCCCTATCGTATCCTCCACCCGGTTTGCAGCTGGAACCAGTTCTGTGCCATCTCCGAGAGTGAAATGGGTTCTCCTTTCAGATTGTGACCTCAGGACCCTTTGTCACTAGTTTTGTCGGCTACTTAAGGTGCATTATCTACTTTACCGCAGTCCTAGGCATGTCAGGGGCATTAACTCTGTGCACAGCCTTGCTTCTGTTTCAAGAGTGCCAGCCCTGCTGGGGCATCTGTTCTCCACCCTGTGCCCAAGTTGGCCTGGACACCCCACTCCCTGGGGCTGCTGGTTGGTGCTGCCAGCCTTCCTAGGTGTCCCAAATGGCCCTTTCCCAAGAGCTTTGTCACAGAAGGGTTCATGGCCAGCTCAAAGTGGCCCCCATATTCAAGCAACACCTTGACTCCTGGCATCCACAGTGGGAGCCCCACTCTTCACTTCTTGAGTCGCCGTGGCCCAGAGTCTGTTCCTGGGTTTTGGGGCCATTATGAAACGAGAATCCACAACAGCCACATTTGCTCAGGCACACATGGGACAGTTTGCCAAATGGGTGCATGAAGTGCGGGAGTCATGGAAGAAGCCTCCACCTGCCCATCAGGGTCTCAGAGCCCCTGCCTCTCTGTTCTGCCTGACCAGTTTCTTTGTATGGCTTTGCACCCCTCTCCCAGGGCCTTCCTCCTTCCAAGTGACCAGCGGATTGACGTGGAGCTCTGGGCAGAGCAAGCAGAGTTGAATTCCACTGAGCTCCACCAGATGCGCGTGCAGGACAATTGCCTCTTCTCCATCAGCCCCAAGGCTGGGAGCCTGAGTCCTGGGCAGGAGCAGATGGTGGAGTTAAAATACAGGTGTTCCCCACCCCCCAAACCCCCTGCACTTCCAGTCATAGAGGCCCCTACCCCGGGCATTTCATTGTATGACTCCAGGGCACCCCAAATGGCCATTCCCTGGGAGGACCACCTTCTCACCTGCAGAGGAGGTCTAATTTCCTTCTTGCCCCATCCCAACTCAAGGGCGAGTTGCTCCCAGGGGCTGGCCAGCTCAAAGCCACAGAAATGTCATGTCCATTTGCTCTCCTACTTGTACCAGGGCTCCAGAACTCACTCCCAGACTTATCTGTTTCTTTCTAGCCACCTGTTCATCGGTACTGATCACCTCCCAGTGCTCTTCAAGGTGTCCCATGGCCGGGAGATCCTGGTGAGGCCCCAGCACACTGGCCCTGCCCTAGTTTCAGGCTGGAGGAGGGGGCTGGCTGGACAGGAGCAGGGAGCTGTGTTCTCCTGGGGAACTGGGGTCCAGATCTCCACTGGCTGTGTCAGTTCCTCCGGGGGAACTTTTATTTCCAGCTAAATTTCATAGGTGTGACAGTGAAGCCGGAGCAGAAGTATGTGCACTTCACCTCTACTACCCACCAGTTCATCCCCATTCCCATTGGTGACACGCTACCCCCACGGCAGGTCAGTGGTCCACATTGTCCTGTTGACCAAGAAGGCCTAAGTGTTGATCTCTAGGTATGAGTGTCAGCCCTTGAGAGGGTGACAAAGACATGGCCCTCTGGAAGCATTGAGGGGCTGGGCCAGCAGGTTGAACCCCCAGACTGGGCAACTGGGAGAGACTTGAAGGGAGAGGCTGCAGTGTCCTAGTTCCTGGTGCTAAGCCCTTTGCCAGGCCCAGTTCCAATTAAAAAATGAGGATTTTTTTTTCCTTTAGCAAAATGTTCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCTCCAAAAAGGATGTCCTTCTGGAATGTGAAGAACTAGAATGTGAATTCTTGGGAAGCTTTAGACCTTTCTTTCTTTACTGATTGAAGGCAATACAGTTTCCTTGCTCTGGAAATGTAGATTGGTAATCTCCAAGAGGGGGACATAATATTTTCCCAAACATTTCACAGCTGTGCAATTTCTAATCATTTAGAAGCCCATGTGTTTCTGAGCAGGGGCCTCCAGCCATTCACCCTAGAGGCCAAGGAATGGGCTTCCTGGATCTAGAGGGGAGTTTCCTATCCCCAACTCTGTTCACTTCCATACCCACCCGCTACCCCTCTGCCTTCCCAGCAGCCCCACTAGGGTCTGTGACAGTGAAGCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCATGCTCAGGAGTGAGTGCTCAGAAAGGGGAAAAGGGGCTGGGACTGAATCCAGATGGACTCTATTNNNNNNNNNNNNNNNNNNNNNNNNNNNNNNNNNNNNNNNNNNNNNNNNNNNNNNNNNNNNNNNNNNNNNNNNNNNNNNNNNNNCCAACCTTTGGAGTTGGTCTTTAGTCCCTCAGACTTTTCCGCCCCCTTACTTGCCTTTAAGCAGAACTCCCTTNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAAGAAAAAAAACTTGATGAAGTTAAACTGAAGACAAATAAAATAAGCTACTCCTTTACAAATAAGATTACAGATTATCCTAAAATATCACCGGCTGACCATACAGTTACATACTTCTCGAGTGATCAAGCCACACGGGAGCTCACAGACTCAAAGGGTGACATCATGGAGATCAGCCTGCCCTCCCACAGTGGGCACAGCATCCCAGGCTTTCTGATTTGCAGTGCTGAGCTGATGTGGCAATTTTCCTATTCCTGCCCCCTGCAGATTTATGAGCTGTATAATGGTGGCTCAGTGCCCGTGACATATGAGGTCCAGACCGATGTCCTGTCACAGGTTCAGGAAAAAAATTTTGATCACCCCATCTTTTGCTGCCTCAACCCCAAAGGGGAGATCCAGCCAGGCAGCACTGCCCGGGTCTTGTGGATCTTCTCACCTATCGAGGCCAAGACCTACACGGTGAGCAGCAACCTGGATGACGTGGGAGGTGGGGTGGTGCTGGCTTCCTCAGCAGGCCTCCCTCTGACCAGTCCCTACCCCCACCCTCCTAACTCCCCAGGTGGACGTGCCCATACACATCCTGGGATGGAACTCGGCCCTCATCCACTTCCAGGGAGTGGGCTACAACCCCCATATGATGGGGGACACAGCCCCATTCCACAACATCTCCTCGTGGGACAACAGTTCCATACACTCTAGGCTGGTGGTGCCTGGACAGGTGGGGAAAGGGGAGCTAGGAAGGCCTGAGGCCTTGTGGGAGAGCCTCAGAGCCCAGGGTGGCCAGACAGAAGGATGGGGAGGCTGGGAGGGACATGAGATGTAGTTGAGGAGGGACAGAGGCAGAGATGAGACCTTGGAACTCACCCTCCTCCAGATTCAGGAGGCAGCACCAGCTCCATGAGAGGTCTACCCTGCCACCTAGTGCCCACAGGTGTTCCTGCTCAAGCCGCTCCACCTGCCTCAGGGCCCTCCATCCCACCCTCTTCACTGGGATGAGGATGTTAAGAGATGGCATAGGAGTGGGGGTGTTGAAAGGTGTCAAAAGGGCCAGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGGTTCTTATTTCTTTACCTCCGCTCTCCACCCACAGAATGTCTTCCTGTCCCAGTCTCATATTTCCCTGGGAAACATACCTGTGCAGAGCAAGTGCAGCCGCCTGCTCTTCCTCAACAACATCTCCAAGAACGAGGAAATTGCCTTCTCCTGGCAGCCAAGTCCTCTAGATTTTGGGGAGGTGAGAGTCCCCTGAGCCCTGGAACCTCCATCAGAGCTCCAGACCCATCCAACCCCACCTGACNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTCCGTGCCATCCTATCCAATTCCATCCCTGTGAATCTCCAGAGACTCCTTCCCCAAATCAACAGGTGTCTGTGAGTCCCATGATAGGGGTGGTGGCTCCTGAAGAGACGGTCCCATTTGTGGTGACCTTGAGGGCCTCTGTGCATGCCAGCTTCTACAGTGCAGACCTGGTATGCAAGGTAGGAACCAGCCCCAGGGGTAAAGTGGGATGGAGGCATGGCTCCCAATGCTGGGGTGGGGTGTGATGGGCTGTGAGATCCCCAGACCCCAGCAAAAGGCAGAAGTCAGCCCTTGGAAGGGAGAGGGCTCACAGCATAGGCCCCGGGCAAGCTCCAGACCTTGGCCTTCTCTCTCTCTGTCCTGGGCCATAGCTGTACTCGCAGCAGCTCATGAGGCAGTATCACAAGGAGCTGCAGGAGTGGAAGGACGAGAAGGTGCGGCAGGAAGTGGAGTTCACCATCACCGACATGAAAGTGAAGGTGAGGAGGGCTGAGTAGATCTCAAACACCCAGATCTTTCTGACTGGCCTTACCAGAGGGGACAGAGGTAGTGGGCCAAGGTGGCCCTCCAGGCCAAACCAGCCAGCCTGCCCTAAGAGATTACATTTCATTCACTGGACTCAGCTTAGCATCTCCACAGCCATCCGGAGAGGCTCTCTGGGTAGAGAAGGCTGGCTGTCCATTCCCTGCCTCGTGNNNNNNNNNNNNNNNNNNNNNNNNNNNNNNNNNNNNNNNNNGAGATTTAGGGGAGACAAGCAGAGCTCACANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTAGAGTTCACCTTCTGGTGGAAGANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGGGTACNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAGTCTANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGTCTAAAAATACTTTTTNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGTGTTTTGGGAGGGGGAGGGTCTGATAATAAACACGAGACAACACTTCCTTCTTGGTTCCTCCCCCAATTTCACAACCTTCCTACACTGGTATTCTTGCTCCTCAATCACCTTGGTGTCTGCCACACTGAACCCCCAGAGACTCACTGCTCCCCTCTGAAGCTGTGGCCCAACTGTGGATACAAATTACTTCCTAANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCAAAAACAACAACAACAAAAAAAACTCTTCTTGGGGGCATGAGGGAAGGAATTTCTGAATTAAATCTTAATGAGGGAGACTGATTTCTCATCTCACTGAACCACAAAGCTCCAGGGCACGAGGGCAAGCTCCCTTGGAGCTGCCCGCCTACATTTGATGGCATTTCCTTCACTCAGGAAACCAGAATTTCCTGGTCTGCAGGCAGCACTTCTCTAGGGAAGGTTCTAGAGGAAAATGTGACTGGGAATTGGGCAGGACCAAGAGGGCAGCAAGAGGATCAAAATGGGGGAGTCTCTTTAGCCTCTCACANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGGGCCTCTTGTATCTTAAGCAAGGATCTCTCTTTTTCCACAGAAGAGAACATGCTGCACAGCCTGTGAACCTGCGAGGAAGTACAAGGTGAGGCGGCGAGCCCCAGGCCCATCTTCTTGGGACAACTTGGGAGGGTCCTTGCTTGCTCCCAGAGAGACCTCCAACCCCTGGACCAGACTCCCAGAAAGGAGGGATGAAAGTGCCAATGAGTGGGGTCACACGCTTATGTGGCCCAAAGAGCCTGTGGGGGAACTAGGTCAAGGGACCCCATTGGTGATGTGAATGTGGTGCCCAAACCCTTGTCATCCATGTCCCTCCCTTCCCAAAGACACTGCCTCCCATCAAGAACCAGCAGTCTGTCAGCCGGCCTGCCAGCTGGAAACTGCAGACCCCAAAGGAGGAGGTGTCCTGGCCCTGCCCCCAGCCACCCTCGCCAGGCATGCTCTGCCTGGGCCTTACTGCCCGAGCCCATGCCACCGACTACTTTCTGGCTAACTTCTTCTCAGAGTTTCCCTGCCACTTTTTGCACCGGTAAGCTCTTGGGAAGGCAGGGGTCACCTTCCTCTTCTCTCTGTCCCCTCTTCCACTCCAGAGCAGGGGCTGCCCAGACCCAACCCATGGTGAGGGGTGGAGCATGGGCAGCAGCAGTGGGGCTCATTGAGCGCCTCTGGGCCCAGGATGCCCCATTACTGCTTAGGCCCTTTTAGGAGGGGGACAAAGCAGCCAGAGCCCCCTGGGGAACACACCTAGGCAGCCCCTACCCCTCTTTGTTCCTTCTTTTTCCAACTGGGAGTCTCTCTAGCTCCCGTGCTAACTCTGAGGGTTTGAGCTTGGTTCTCTGAACCCAGTAGCACTACATTTGGGTTAAGGCTGGGAGTGCCTCAAACTCATCTGTGTCCTCATCACTGGGCAGCTACCACCACTGCTTGTGCCCCCAACCCCAGCATGGTGGCTTGCCCAGGCCACTCAGAATGTCATGGCTGCCACAGGGAGCTGCCAAAGAGGAAGGCCCCCAGGGAAGAGTCAGAGACTTCTGAGGAAAAATCCCCTAACAAGTGGGGCCCTGTTTCCAAGCAGAAGAAGCAGCTCCTGGTTGACATTCTCACCACAATAATCAGGTACCAGCTCACACCCTGGGCCCTCATTGCCACCTTCGCATCCCTCCAGCTCATTCAGCCAGAGCCCCTTACATTCCAGCACTCTTCCTTTCTTGAGAAATAGAACTTGGTGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAGAGACNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCAGAACTTCTTTGCAAGATCCCACCACGGTCCCCCCCCCCGGCCCCCCCACCCCAGGACAGTGGGGCTCTGATGGAGCTGGCTGGAGGGATTCTCAGGTCTGGATCCTGAGTTCTGCCCCATTCCAGGGCTGGCAGATGAGTCTGGAGCCAGTCCACTATCTCCCCAGGGTTCCTGCCCATCATGTGTGTGAATCTGACCTGCCCTGCCTCCAGCATACCATGTTCGCTTCTAAAAATCCATGAGGCTCAAAGATGTCTTCCTTGAAAGAAGGGACAGGAGGAACAACTGACCAAGGGCTCCCCTCTCCCCACCTAGCAGCCCCTTTCCCACCTAGAACCTAGCTCTGAAGCCAGGGCTCCCCAGGGGCTTCAGGGCCCCTCAGCAAGGGCCTTCTCCTTCTCCCTGCCACCTAGGGGCCTGCTGGAAGACAAGAACTTCCATGAGGCTGTGGACCAAAGCCTGGTGGAGCAGGTGCCGTACTTCCGCCAATTCTGGAATGAGCAGTCAACTAAGTTCATGGACCAGAAAAACAGCCTGTACTTAATGCCAATCCTGCCTGTACCCTCCAGCAGCTGGGAGGATGGGAAGGGCAAGCAGCCGAAGGAAGACAGACCAGAGCACTATCCAGGGTTGGGAANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAAAGTGAGCTGGGCGGGCATCGGGCCCACACCACAGCCTGAGTCCCAGGAGTCCATGCAATGGCAGTGGCAACAGCAGCTGAATGTCATGGTGAAGGAGGAGCAAGAACAGGACGAGAAGGAGGCCATCAGAAGGTGAGCAGCCAGGCCCCAGTGAGGACGGGAGGGAGGCGAAGTGCAGAAGGTTCCTAGGAGGCAGTGGGATGCCAAGGATGGCTGCTGCCTGTANNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGGAGAATTGTTATAGTCTGCAAAGTCTTGTAAATGGGCTTCTAGAGCCAGAAAAGGACCACCTGTCTTGCAGGTGGGACTTCCCGCCGGGGCTGCCCTACAGATGATTTATGTCATTGTAGGGATTCCCTAGGCCCCACCCAGTCCCTTCTGCGCCCTCTCATCACTTCCCTACCTTTCTCTAAACTTGGCCCTGAGATGCCTACTACATCGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGCGAGCGAGGAGGCGCGCGGGCACTGCGGCCGCCCTCATGCTAGCCCCCGCCCCCTCGCAGGCTCCCGGCCTTCGCCAACCTGCAGGAGGCGCTGCTGGAGAACATGATCCAGAACATCCTGGTGGAGGCGAGCCGCGGGGAGGTGGTACTCACCTCGCGGCCACGCGTCATCGCCCTGCCGCCGTTCTGCGTGCCCAGGTAAGGGCGCCGGGACCAGCGGCCGCGCAGACCGCGCGAGAGGGGCCACGGGGGCAGCCAGGCTCTGCCCGAAGCCTCGCCCCCACGCCTACCTGGGACTTTTTGTCTTCCAGGAGTCTGACCCCGGACACGCTGCTGCCGACGCAGCAAGCAGAGGTACTCCACCCGGTGGTGCCACTTCCTACCGACCTTCCGTAAATGCCCGCGCCCAGCCTCTCCGACATGCCGCTAGGGGTCACGCCTGGCCCCCTCTCCACCGCCAGTAAAAGCATCTAGTCTTTTTTCTGTATCTACCTTGACTTTATTTCCCCGACGAGGGGGAACGCGCGCGCGAGCTCTGAGAGGGAAGGATGGCAGGGAGCAGCCCGGGCGGACTGCAGAGGCGCGGCGGGAACTTAGGGCCCGGGAGGATGCACCTCTCCGCCCACACTGCAGCTGGACTGAGACCTGGCCGCTAGCTGCCGGCCGCTGGACCGGGTCTGGCCAGTGGAGGGCGCTCCGGACGGGGCCTGGCTGGCCTTTGCCCCAGCGCACCTTGGTGCACAGTAACTGAGTGCCAGGCCTGGCCAGCGAAGCAGGTGTCCACTGGGCAGGGCGGCCACCCCTCACCTTTCCGCGCCGCGCCTGCTCGGAGGCTTAGCACCCCCAGGGCCAGCCCCTTAGCATTTGCCGAGCTCCGAGTGCCCAAGAGGCTGGGGAGGAAGAACTGAGGCACGCCGCGGAGGGCCAGGAACTAGGCTGGGGCGGGTCGAGGTCACCACTGGATTTCGGGCCACAGACCCTGCTAAGCGACTCCCACTGTGAATCAGTCCTCAGACGAGGATGGATACCCACCTCTCGGTTCCTCCTAACCCCCGCCCGGGAGCTTCTGTCCTCTGCTTCTCGGCTCCTCCCTCCCAGAGGCAGGTGGGTGGAAAAGTGACATCTGGTGTTGTTCCAGAGGCGCCCAGGGTCCCCAGCACCCTCCCCTCCGCCCCCGCCACCAAGGCCTCGGAGAAGCTCCGGTCTCAGAGCCCGGGCGGGGGAGGGGGGAGCGCTCCGCCTCCATGAGTCAATATTTGCCCCGAGCAAACGGGCGCCTGGACAGGTGTGAGTGTGTGTGTCTGGGGGCGGGGACTGAACAGGCAGTATAAGAGCACACGGAGCCCGGGCTCCGGGACAAGCTCCAAGGCGTGGGCTGGGGACGAAGCCAAGCTAGGCTGGGCCGGGCGGAGGGGCCAGGAAAGCCGGGCGGAGGGTGGCTGGGAAAGGAGGGGCGGGCCAGGTGCGCACCTGCGGTGGCCGCCGCGGCCGCCGACGTGTCAGCCGCAGATGCGTTCAGGTGAGGGCGGAGGCTAGCGGGGCGCTGTGCAGCACTGAGCTCGCGGAAGACCAGGACCAGGAGATCACCGAGGGCGACCGCCAGGCCCCGGGCCCTCCGCTCCCGCCCCGCGACGAGCCCCTCGCACAAACCGGACCTGAGCGTTTTGTTCGTTCGGCTCGCGTGAGGCAGGGGCGGCCTCTCAGCACCAGCCCGGGGGCCGGCCTGATCGCCACGCAGGCACCTGCCGCCGCCACCGCCACCGCCATCTCAACCGTACGGGTGGGAGAGGCTGTGCGCCGCTCCAGGGGAGATCCGGCTCCCATCCGGCCCCACCCGCCCTGCCTTGCCCTGCCCGCAGCTTCTGGGCTGCCAGGCTCCATTCTGCGTCGTAATACGGGCTGAGCCGGCTCCAGCGGGTGGCGAAGGGGGCCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCCTCCCGCAGGGGCTAGGCCCTCCCCAGCCTCCTGTCCCGGTTCCTGGGCTCCTGGTCATGCTGGGGAAGATGCAGGCCCTCACGGAGTTGGAGCCAAGGCTGAGGTGTGGAAAATCTGGGCGGGGGAAGCTGAGGGCCACCATGGAGCCTCAAATCCACTCCTCGGATCTCTGTGGTGCTGCTGACATCCTGAGATGTCACTCCCTGACACTCCCTTGGTCCCTCTTTCAGTCTAAAAACGTCTGAGACAGTCAGACTGACTGAAAAGGACAAGCCCCCAGGCACAGCGGTTCACCACCTTCCTCAAACCTCAGAATCCCACATCCTCGCTTCCATCAGCTGGGGTGGGGTAGGTAGGTGGCCCTCAGCTCAGGCAGAGGCAGCCCCGGGAGATGGGAACCAGGGTCAGAAGTCCCTCCAAACTGCATGTTCCTAGAGGTGCTGGATTGTCCCCTCCACCACCACCCGCTCTAGTGCCCCTATGTCACCCAACTACCCAGAGGCCCCAAAGGATCTGCTGAGCCCGAGTCCGTGGCACAGAGAGAGAAGGGGGAGGGGCGAGATGGCGTTGGACAGATCCCAGGCAGGTTTAGGGAGAGGGTGGACTACAGCAGGGCTCTTCGTGGAGCAGCCCCAGGGGCAGGGGAAGGTCCTTGGGTCCTGGGCTAGGGTGTGTGCAGCCCTCTCCTGGCCCCCCTGACCAGGCTCTGTCCTGGGCCAGCCCTTTACTCTGCCTGGCACTGAAAGATTTGCCTGTCAGGGGCCTGGCATGGTCCTGGTGCACCTTCCCTGTGTCTTTGAGGGCTTCCCCTTCCCTCCCCAACCATGTCAGGGGCCAGGATTCCACATCCTGGGTTTGCACAACAAAGAGATCTCTGCTTGGAATGACCCCCTCCCAANNNNNNNNNNNNNNNNNNNNNNNNNNNNNNNNNNNGCCTTGAGGGGAGGGTGGTGAGAAGGAGGCAGCCACCCAGTGGGCGGGGATCTTTCCTGGTGACTGAGAATTACTGCCCCTTCACCCCAGGGCCTAATTTCCCCAGTCCCCCACCCACTATCCATCCTAAGACTGCAGTTGCTGGCCATTACCAGGGATCTGGCCTCTCATCCAGGTCCCTCCTCCGCTGCTCCGCTGCCACAGGGCGGGGTCTCCCAGTGCCGGGCAGGCCTGCAGGTGCGGGGCTGCATGGGGAGGGGGGCACTCAGCAGCTGCTGTACGAGGCAGGCCCCCTCCCCCTCCTGCTCAAAGCTGGAGCCTGCTTCCTGTCGTCCCTGTCAGCACCCTGGGTGGGGGAGGGACCAGGTAGTGGGGGAAGTGGAAAAGGGATTGAGCGGTTGGAGTGCAGCAGCTGAGAAACAGCAGAAGAGAAATGGAGAAGGATGAGAAAGAGACCAAGAGCATAGGCTGAAGGAAAGAGGGGTGTGAGAACACAGGGGAGATCCCAGGGGCTGCAGAGGCTGCAGACCCTAGACTGTGAGAGCGAGACCAGAGGCAGAGATGACCAGAGAGAAACCAGGGAAGAGGAACAGAGAGAGACACATGGAGACAATTCAAGATAACAAGAGATGTACGCTGAGAGAGATTGGGGATGTGGATTCATGGGGCTAGGGCTCAGGGGACCAGAGGCGGGAGGCATCGTGTGTATGGAGAAAGGGGGAAGCTGCAAAACAGCCCCATCCCACCCCAAACTAGGGTGGGAATTAGCCCCAGCAAAGGATAAAGAGGTGATTTCTAGGTGGCAGACAGGTTTGGAGGTGGGGGGAGGGTGATACCCTTATCTTGGAAGCCCATCTCCTCCCATTAAAGGACAGAATGTGAGGGAGGGGCAAGGCCCAGGCCGTGGCTGGACACCAGGGGCCAGTGAGGGGCACAGATCTGACTCCATCTCTCTGAGTTTCCCTCTTGGCCACAGATGGACAAGCCAACAGCTATGGCAGCTGCCCTGCCCCCACCCACCCCAATTATTCTGGCCTAGCCTTGGAATTTCTGGGCTTAGAGGCCGCCCAGGGTCTGTGTGACAGTGGAGGGACCTGGGAGTGGAGGCACTGGCTGGCAGGGGGGAGGCTCACACCCAGGTGCTAATCCCCATCCGGCCAAGGCAGTACCAGAGCCTCACTGACCTTCTTGGAGATTAGTCCCCATGGAGACCATGGCAACTCGCCCCCTCATTCTGGCTAGGCCAGACGGGTGGGTGGGTGGGGCAGGCATACGCTGGATAGGGAGGGGAAGGAACCTTCCAGAGGCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTCGGCTCGGTTCCCAAGCATTTTTTCCTAGAGAGCAGAGGCCTGAGCAAGAACAAAGGAAATTGGCACACTCCTCTCTGACCCTCCCAACCTTCCCAAACTGAGGTCATGAACTGAGGAGTGATTCAAATAGGGTGAGGCAGGAAGGCGCTCCGTCCTGAGCACTAGGCTGGCTTCTGCCAGGAACCAGGGTGAGAAGGAGGGTACAGCTCCCAGAGCCATGCTTGCAGCCTGACCACCCACCAACCAGGCGAGCGCCAGGGGTGGTGGGAAGCAGGTGTGTCAGAGCCAGTTATAACCACAACCACAGCAACAGCGGCGGCGGNNNNNNNNNNNNNNNNNNNNNNNNNNNNNNNNNNNNNNNNNNNNNNNNNNNNNNNNNNNNNNNNNTCTTAACCCACTCTCTTCACCCTTCGGTGTCTGTATGACCTTGCGTGGGTGGCTGAGCTGATGCCTTCCTGGGTGACTGTGAAGTGGGTATGTGCACATGTGAAAGTGATGGGACATATTCGTGTGTGAGTNNNNNNNNNNNNNNNNNNNNNNNNNNNNNNNNNNNNNNNNNNNNNNNNNNNNNNNNNNNNNNNNGACCAAGTGTCTGGCCATACATGAATATGACATTGAGGTTTCTATGGAATCCTAGAATGAGACTATTCACATGCGAAGCTTGCGGGATCACATCTATGTGCCTGGAACTGTGGATGATTCTGTCTCCATCTGACTGAACGTGTCTGTGTGTGCCCTCTCCCTGTCACCCTATGTCACCCCTGTGGCTTTGTGTCTGGGTGTGTGAGTGATGGTGAAAGCTGCCTGCCAGCTCCTGGATGGTGGCCAGTTCTCTAACCCCACTGAGGGGCAGAGGAGAGGGGCTCTGTTTCTAGTGGGATAGAACAATGGGTCTGTGTGGGTGGGACTCCTAACTGGCCCAAACTGGGCTGGTCTCTGTACCCCGTTATCTCTGTCTCCCAGGAGACCAGTCCTGCACAGCCCCAGGGAAGGTGCTCATCTGCTGATTCTGGGCTCCAGACTGGGGGTGGGGGCGGGGGCCAGAGAAGCAGGGGTGGAAAGGAGGGGCTGGTGATGGTGGCAGGGTGAGGGAAGGCACCCTTCCCTGAATTTGCATCTTCAGTACCCAACCCCTGGATGATTTCCCAGTAAAACGAGGGGGGAGGGCTGGGAGGCGACCCGCTCGAGGAGGGGACATTGGCCACCAGCCCCTCTCCGCTGCCCAGCAGTACTGAGCAGTCAGGCTGTTCCTGGGGTGGGGCTCTAGGTGAGAGAGGTGCCACTTCTTTGGGATTCTCAGGGCCTTTGCCTAGGACGGAGATTGGGCAGGAGCTTAGGAGTGGGTTTGAGGGCTTCCTTGCGGGATCTATGTGTCCCCACATGGGATGGGAGTCCGGGTGGGATGTGAGGGCCTCATGGAGGGGACTTAGGCTCAAAGGATTCTCTGTGGGATCTTATGGGTCTGTATGGGATGGGGGTTTCGGTGAAATCAGAGGGCAGGAGATGGTGGCTAGGGATGGGGTGAGGTCCTGAGGATTTCCTTGTGGGACTCAAAGGTCCCAGTGTGAGATGGAGAACTGAATGAGGTCAGGGTGGGGGCCCTGAGGGTGATAAGATCTCTGTGAGATTTTGAGGGCTCTGTGAAGTGTCCACTGGGGTTTCTGGTTTTTGTCTACCTAATTCCCCGCTCCATGCCCCAAGGCCAGGCTGTGGATGTGGAGGGGGTAGGGGTGACTGTGCTGAGCTGGCAAACAAAGCCACCTGGGCACCCATCCTTCAGGAAGCTCTGGGCGCAACTGATCTGGGAAAACAAACAGGAACAGCTGCTAGGGGTGGGGGGGGTGGGCGGCATGGCAGCTGGGGAGAGGAGCAGAGCTGGACCAGCCTGGTAGGGAAAGGAGGAGACCAGTAGGGGGTCCGTGGTACCCAAGGGGGGTTATCGATCCCAGAGTCCTCTCCTGGCACAGTAGGCAGGGCTGCCTCGAAGCCCCTTCCCCTCAGTGGTTCTTCTCTTTGGCGATAACTGTGAGGCCTAAAGTCAGGAGGGGCCTGAGGGCGTTAGGGGAACCCTGAGATGTTTAGAGGTCTTCTGGGCCAGTGACTATGGGTGGCGGTGGTGGGTATGCCCATTTCTGAGAGAGCTGGTCGCAGTTACTAGAACTAGAAGTCATGGGTGAGGGGGTGGGGGGGGTACCTCCATACCATTTCCACGGGGAGGGACAAGGTAGAGAAGGGGAGAGGCTGGCTGCCGCCAAGACCAGAGTTTCACACTGGAGGAGGAGTAGGGGCCCTTCTCACACACGGGGTCCACTCCCCAACTCCAAGCGGGACTCCTAATTCTAACCTGGACCTCGGCTGGACGGCCTCCCCGCTTTTCCTCCTCCCCTAACCCTGGAAAAGGCGCGACGCACCACTCCTGCAGAATGCCGCCGTGGGACCCACGCGTGGAGCCCGACCCTCGAGAGTACGTCCAGCCCCCAGCCCCGCTCGGTGCACAGTGTCCAGTCTCCGACAGACACCCCCACACCCTCTGACCAACACTTGGCGTCCGCGTCAGGGACAGCGCAGACGCACAGCCTGACGCACTAGCCACCCGCGCCCGCGGCCCCTCCCGACCTGGACCTCCCCTTCCACCCCGCCCTCCGCGCCAGCTTTCGGGCCAGCGCGGCCTGCTCCTGCCCTCTGGTGGCCGAATCCAGAATTGTCGGATAGAGATGGGGGAAGGAGGGAAGGCGAGATCTGATTCTTCACCCCTCACCCCTGCCCGGGCTGGTGACACTGAAGGCAAAGACTGGGACACCAAGGGTCCAGAACTGGCTCGTGCCCCACTCTGTGGTTTGTATTCCCTCCTTGAGGTTTGTGTTTGTTCAAACCCCCGCTAGGTAGGGAATCAGGACACTGTTCTGACCTCACCCCCAGTCAAACCTGCCTGAGGTTGTCCCATCTCTGACCCATATCCTCCTCACTCGGACTCTACCGCCATGCCCAGTTCTAGGGCCCTGTACTGTTGGCACCCATCTTCACACACACTGCGAGCTCCAACAGTAACTCCTCACCAGGGAAAAGAGAGCCCAAGGGATCCTCTGTCTGCCCCCAGCTCCTGCTCTCAGCAACCCAGAACTTCAGAGGTGGGAGAGCAAGCTGCCAAGACCCCCCCGCCAACTTCCATTGGAGAATTTTCTCTACACCCCTTTTCCCGTGGACATTTGGCCCACGCCTGACCACCTTGTTCGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAAGGCTTCTGGGAGCTGGGGTGTCCGCAGCTGGCCTGTGAGCACTTTTTGGCACGGGAGCTTCAATGGACAGCGGCGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCTCATGGAAGCAACAAAATATTAGCTCCCTTCTCACTCGCCCCTCTCACCTCTGCTGGAATATCCCCACCCGCACCCACCACCATTAAGGAAAACCTACAATTTTGCAAGAAGCCTTTGTGTCTCATTTGCTGTGGTTAGAAAATTCCACTGCAAGTGGTGAGGAAATTTCCTACCTGGAGCTACCCCTTGGGACTCCCAACCTTTTTGGAGCTCCCAACCTTTTCCTCTTCCCCCTCTACCTCCCCTTTTCTCTACACNNNNNNNNNNNNNNNNNNNNGCCGCCTTTTCCCAGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCTGGGAGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCCTCAGCTGCTAACTCTCAGGCAGACGTCGAGGCTGGAACCCTTAGAGCCTGGAGCTTTGTGGGAGACCCGCAGCGGCACCGGTTGAGCCGAGCTAAAGCCCAACATTCTTCCTACCTCCCGCGGCGAGCCACTCCCTAGGTGGCTATTATGCCTAAACACTGAGTAAACCGCGCGGCCCTCGGGCGGTCGTTTCCAGCGCCCGGCGCCGCCGCGTGGCGAGTCCTGACCACCGCCTCCTCTCCTTCCCGGGCCCGAGGACACCCCGGGGGCGCGGCGCAGGATCTGGGAACGCACGCTTGGGAGGAAAAGGGCCGCGATGGACCGATCCCCCAGGAAAAAGAGGACTGAGCACATGAAGCGAATCCTTTGGGCAGAGCCCTCTCTACCTCCGCCTTAGCATCGCCCAAAAGAGAGGCCTGGACTAGGGCCGAGGAAGGCTGGCGATTAGACTCTGGGAAGAACTTCCGCTCAGCGAGTCAGCAGCGCGGTCTTAGCCCGAGGCCCAGGGGAGGCCAGGTCCCTTCGTCCCCGAGGGCCGGGACAGGGAGGGCGCGTTCTGCCCCGAGGCCTGGCGTGCCCTGCCTGGCTCAGGCTGGAAAGAAAGAAAGCGCGGAGTCAGCCTGGGCTGCGGCGGGCGCGGGGTGCGCGCCTCAGCACTTTCTGTTGTCTTTCCCGCGGGCTGGGGCCACAGCGGCGGCTGCGGGAGAGGCCTCGGCTCCCCAGCGGAGACCAGAGCCGACCGCAGCCTGCCTGCTGACTCAGCGACCCCACCCCCGAGGGAAAGGGTGCGGACAGGGGGACAAGGCCCAGGTATAAAGCTCGGCCCGCCCGGCCGCGCGCAGCCTCGCAGCCGGTCCATTTCCCTCGCGTGCCGCGCTCACCCACCCGGTGAATGCCCTCTGCTGGGGTGGGGAAGGGAGGGGAGCGCGGGAAGACTGCGAGCCCCTGGCAGCCGGGTCCAGAGCTAGGCCGGGTCCAGCCCCCGGGTTCTTCATCCGCTACCCAGGTTGGGCAGGCTGTGTGTGGCTCGAAACGGGGCTCTTGAGACGCTGAGCGCGTGGTGCCACCCTTCCTGTCCGCAGCGGCATGAGCANNNNNNNNNNNNNNNNNNNNNNNNNNNNNAGCCTCACGCTCTGGGACGAGGAGGACTTCCAGGGCCGTCGCTGTCGGCTGCTAAGCGACTGTGCGAACGTCTGCGAGCGCGGAGGCCTGCCCAGGGTGCGCTCGGTCAAGGTGGAAAACGGCGTGTGAGTGATTGCCTGGCCGCGGCTGGGCTGGGTTGAGGGGCGCTGGGTTCCACCGGCCTAGGCTCAGAGCCTCAGCCCCCTGTCCTGTGAGCCCTGGAGAGACCTGCCAACATGGGAAACCCCATAGCCTCCCCAAAATTCCCCACCCATCGCAAGATAAATCATGGGAAACTGTGTATTTTTGTGCTAGCTAAAGAGACACAGGCGAGAAGCGACCCCTTGAGGGTCCCTATGGGACCCCACCGTGAAGGGGTTTCTAGTGTACTTACACCCTTCCCAGAGTTTTTCTCTGTGCAAAATTAGAAGGCTCTGCACTTGGCTTAACAAAAGAAACCCAATCCCTTTCCCTGGCCCCACCCTGGTCTCCCAGAGCCCAGCTTTTCCCACCCTGCCGCCCCCTACCCCTGCCCCAGCTGAGGTCCCAGTTTCTGCTGTGATATTCGGGAGGGGTCCTCCAATGTGTCCCCATGCAAAGGCCTAAACCCTTGAGGCAGGAGCACTGGGTCGGGAGATCTTCATAACCAGGAAAACTCTTTCCCAACCTGGGGTCCTTGAGGGGGAAGCCTGGGCGCCTCAAACCCCAGAAAGCAGCAGGTTTTGGACCTTTAGTTCCGAGAGGGGCTGGATTGCCATACAATGGGGGTGTCAGGGGAAGGGTTTGGGGTTGGAGAAAAACATTGTGCTTCTCAGTCTTTTCAATCATGGCCGGCAAGGGGGAGGGCAGAGAGGGTGCGGCTGGGGCAGATACCTCCCAGCTCTTCTCCTTTTCCAGTTGGGTGGCCTTTGAGTACCCCGACTTCCAGGGACAGCAGTTCATTCTGGAGAAGGGAGACTATCCTCGCTGGAGCGCCTGGAGTGGCAGCAGCAGCCACAACAGCAACCAGCTGCTGTCCTTCCGGCCAGTGCTCTGCGCGGTGAGCACAGCCCACTTCTCCTCCCCTGCCTCCTAGCTCCAGGCCCTGGCCTCACCATGGCACCTAGAAAGGCTGTTCAAGCCCTGCATGGGGTGTGTGTAAACAAACTCCGAGATGGGTGGTAGTAGCATTATTTCTCAGAATCAGAAGGGTGTCTCCAAGAAAGTTGGGATTGTGGGCGTAGGCCCGGAGCCCAGAAATCATCACCACGGACAGCGCTGAGGCCATCCCAGTCCTGAGTGCCCAGGGTTCCCCCACATTCCATCGTCCACACGTGTGAGTGAGTGCCTCTGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTGAAAAGAGTCAGTCTCAGTGTGTTTGTGTGTGTGTTATTGTGTGTGTGAGGGTGTTGTTAATGAATCAAATACACCGAAAGAGAGGACCTCATTTTCCATTTGTGCCGACCTGGTTACTAGTAGGGCTGGGAAGTGGAGAGCAGAGGACCCAGCCCAGGCCAGCCCAATTCTTCTGTTCCAAGAGGCTGAAGAACCCTGCAGTGGGAAGGCCGGAGCCAAGATGCAATCAGTATTGATGCCCTGACATGTCTATTCTGATTCTGCCACCCAGTACCTCTTAGCTGGGCACTAATCTGTGAGAGGCTGTGCGAAGGGGCTGGGCAAGGGCTTGGGTAGTTAGTCAATGAGGACCTCAGGGAACTGTCTTGGTGAATGGGACAGATTTCTCTAAGGACCTCTGATTTTGCTCCTGCAAAATGGGCCCATGTCACAAGATGCTCTCTAGTCCTGACCTGAAAGGGTCTCTGGAAATCCTATCCCCCAATCACCTCCTTATCTCAAAGTCATGGGAGGGGCCAGGTTCGGGGGAACCTCTGAGCGATCCATTTCCCAGACTGCCAGGGAGCTGATGGGGTGAGCTCTGGGAGTGCCTGTGGGTCCTAGACAGCTTCACAGCTTGTGAGGTGCAGTGGGTGGGGGAAGTGAGGCAGAGGATGTATCCGTTAACCCTTTGGAAGCCCTGATCTTTCTGTCCTGATCTTGCTGTGCCTCAGAACCACAATGACAGCCGTGTGACACTGTTTGAGGGGGACAACTTCCAAGGCTGCAAGTTTGACCTCGTTGATGACTACCCATCCCTGCCCTCCATGGGCTGGGCCAGCAAGGATGTGGGTTCCCTCAAAGTCAGCTCCGGAGCGTGAGTCCTGGAACTGGAACCAGGGAGACAGAAGAGCAGGGAGGCAATGGGGACCAAGGAAGAGGGTGGAGAAAAGTGATGTGGGGAGGGCGCTAAGAGAGATATTGGGGACTTGGAGGCAAGGGGAGGGAGATGATGGAGTCAGACTGGAAGGGAGGAAATGAGGAGGTCCTAGGAGAATGGGGGGCAGCGGGCAATATGGGCACAGATGAAGGGCTGGAGAACATGAGGGGGTGGAGGAAATAAGGGGTGGGTATAGAAGACTGGATGAGAACGGTGACAGAAAGACTGGAAGCAGTGGGCAGGGGAGGGAGAGAGCCACGGAGGGAAAGTTGAAGCAATGATTTTGGAACTGGGGGTTGGGAGCTACAAAAGGGGGGCTGCTGGTGGAGAACATAGGGTGGGGGTGGGGGCAGAGCTTACTCCCTGCCTCTGTACCCTCACTGGCCTAGGTGGGTGGCCTACCAGTACCCAGGCTACCGAGGCTACCAGTATGTGTTGGAGCGGGACCGGCACAGCGGAGAGTTCTGTACTTACGGTGAGCTCGGCACACAGGCCCACACTGGGCAGCTGCAGTCCATCCGGAGAGTCCAGCACTAGGCTCCACGGCCCCAGACACCTTCCCTGAGGACACTCAATAAAGGTTCCTGAATCTTCCTGCCCCTCTTGCCTCCTTGGTGTCTTGCTTTGTGATCTCCATCCTCATCTTTCTGAAGGTCCCTCCCTGCCAAGCTGACCCTCCCCACAGGCTCCAGCATCCTTGGCTACGAAGTGTAAGCTGGCTGAGGTTGTGTGTGGGTAGGCTCTGGTGGGTCTGACAGCAAAGTGCTGGTAAGAATCTTAGCAATTCTNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAAAACCTTAGTAATTCTGTTAAATGGCACCAGTCCCAAACCTCTCCTTGGTAAGGTCCACCACTATGGGCCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNCCTTACCATGCACTGAAGTTCTGGGCTTCCTCTGTAACCCCAGAGCATTTTCAACCTTTATCTTCACCCAGTTCCCTGCCCAGTAAGGGAAGCCTGAGCCTCCTGCGTTGTCCCATTTCCTCCATTCGCAGGAGGGGAGTTAGTGGGAAACCCCACTGCTTCCCAGGGCCTTCAAAGGGGAGACTTCCACCCTCTGAGCCCAGCCCCCAGACCCTTTGAACAATGACAATCCCTTGCAGTGGTGTTGAACTCCAGCATTTACAAAGCATCTTCATATCCCCCCTCTCAGCTGCTCCATACCACCCCCTCTTCACTCTTCCTTTCCCTCCCTACCCTTCAGAAAACTGGTGAGCTGTAGAAGTCACCCCTCAGTTGCTAAGCCTGGGTTCTGGGCAAGTGGCAGGTGCCCATAGGCATGGCAGGGCTGAGTATGGTGGCAGCCATTAGAGCCTCTGGCACTAGTATGGGTTTTTTCCCAGTTCCCTCTTCGGTCCAGTTGGTGATGCCCCCCAACCCCCACACAGACAGAGCCTCTCAAGATTCTTGGGACTGCCTTCATCCTGTCCTTCAGGGATTCTTGTGTTTTCAAGTCCCCAGTGGACCTATCAGTGGGGAAGGGTGGCTCTAATTATTGCCCCAGGCCAGGCAGAGCAGGACAATGCCAGGGAGCAGTCAGACTTGGGCCTGAAATCCCCAGAGGTTCAGCTGTGCAAAGTCAGGCAGCTGCTCCTTTGGGAAGAGCTCCGGACCATAAGTCAGAACACNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTGTTTGTGGTGTTATGGAAAAGCACTGGGGTAGTCAATGTAGGGACCTCCTTCCCCTACCTCCCCGCTATGGATCCTAGGGTTGGGTCAGGGGACCAAGGGAGTGAGCTTTGGATGGCGTATAAATTTTGCTTTACACTCGAATGGGCTCTAGAGTCCATGGCCCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAGGTGGAGGCTGAACTGAGCTGTGGCTAAAAGCCACAGGCCTCAGCAGAGCCCCAAACTTCTGTGGGCCCCAGAGGAACCTGGGGTTCCTGGCTGGTCATGGGCACCTCCCCATCCCCTTGTGGAATGGNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNAGCAGGCCACTTCCCCACTGCTTGCTTTGGGGAGCAGGCAATGAGCAAAGGGAGATCTGGAATCTGCCCTTGACTGAGCTGCTCTCCCTACAGGGGTATCTGTGGTTGCTGGGAAGAATCAGCAAGATGGCTTCAGGAACTAGAAGTGTGACTCTGGACCTTCTGGTCCTCCATTCATTCTGGGCAGGCTGGCCATCTGCAGAACCACATGGGACCAGCGCTCAGCAGGGTAGGAAGGGGATGAAGGGGAAACTGCCAAGGACTCTCCTGTTGGCTTCCCTAGCTGTACTTGCAGCAGGAGGGGATGGAGAAGATCAAGACTCAGGTGGGTGGAGATGACTGCCCCCCATGCACACATTTCCTGGTGCTCCACCCCCTCAGCAAATGACTTCTGGGTAAGACTGAGGGATAAAGCCTATACTGTTGATCCTTGCCCAGCTCACAGCTGGGATATTTGGGTAGACCTCACTAGGACCCTCTCCTTTACACCTTAGCCTCCCACTGAGAGCATTAAGTGGGCGAATTTTGATGCCTCCCAGCTTTCCCAGCTCTTACCTCCTGCCCCTGGCTATTTTCTGTTCTTACCTCCTGCTCCTTGTTTTCTGTCCATAGTGGGTGGGGGAAGCAGAAAGAGGAATGAGAAGGAGAGGGATTGGGATCTTGACCCCAACTTCAAGGCCCTCACCTTCTGTGGAGCCCTGAGTTTGGTCCACAGGGAACATTCCAACCTCTAGTCTTTGCTCCCACCAGATTCCAGGTCCACATCCTGAAGGCTTCATCCCTGTCCCACTAGGATCCTCCCAGGCCCTGGGATCCCTCTTTTTCCCTCCACACTCTGCCTCCCACTCTTCTCCCTGGCTCCTTTGCACCCTCCACGAGCAAAGATGAACACTCCAGAAGTTGCAGCAAATGAGGGTGAACATGGGCCCTGTAGAACCCCAGAAGTAGGAGTTTCCAGCCATCCTCAATCAGGCATCCCAGGACCAGGCACTTAGCAGGAGACCTGCCCATTCCCAGGCCTCCTCCACCCCTGGGCTCAAGCAGTGAGAGAGGACTTTGAGAATGCCAGTTTTGACAGTACCCGTTCCATACTGCAAGGCTGCAGAAGGTGGGCTTCTGGGCTGGAGCCTCATCTTCAGCCAGCATCCTCCTTAGCCTGCCTCTCCAGAGCCACGAAGCCCCATAGGTACCCAGAGAGACTCCAGGCCACTTGGGAACCACATCAACATCTTTGCATGAGTTAGAAATAGGAATCCTTTTCTTGGAGTGGATTCCAGCCTTGTACAGTGCACAACCCTGTCTTAACCCCTCCACCTCTCCAGCCTCGTTCAGGGATAGCCAGAATGGGAGTAGTGGTGAGGGTGGGTGGCAGACTGGGAGGACAGGGGATGGGGAAGGATGGGAGCAGAATAGTTGTACAGGGAAGTAGCAGGGGGCTCAGGAACCCATCAGGGAAAGCCAGGACAGATTCTCAACATCTTTAGAGGCCTTCAGCAGTCCAAGCCACTACTAATTTATGGGGACTGAATGAATNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNGCCACCCATCATAAGTTGCCAGGAGAGATGGCCCAGCACCAGGCCTGAGGCTTGGGGGCCCCAGAAAGGCAGGAAACGGGCCAAAGCAAGAAGGAGACGGGGAAGGGGTTCGGGTCTCTAGGGAAGAGCAGAGAGTGGGAGAAGAGGAGGAGGGAGCGGGGATGCGGGCAGAGATAAAGGGAGCAACGGCTGCAGGGCCGGGCGCGCAGCGACAGGCGAGAGGGAGGGAAGCGGAAATTTAAAAGTGAAGATGCAGATAACGCAGCCTGGAGACGGGAACCCGGGTGGGGATAGAGGGAGGGAGGGGGAGGAGAGTGAGCGGCGAGTGGAGCGGGAGCCGACTGGAAGAAGGGCTCTAGGGAGGGGGCTGTGGCTGCTGGGGTCCGAGGTGGGGCCGGGTACACCAGCCCCATCACTGTTTGCAGAGAGTCAGGGAGGCGGAAAAGACACGCGCTCTAGGCTCCCATCAGGGCACATGGCCCGGGCCCATCCCCCGCGCGTCTCCCCGGCTGCGGGGCGCGGGGGGCTGCCGGGTGCGCTTGGCTGTGGCGCGGCGCGTTGGAGACTTTATTGCGATGGGACGATAAGAGGGGCGGGGGCGGGGTCCTGGGGGCCGAGGCGGCAGCGCT

SEQ ID NO: 3 - protein, 340 aa, Rattus norvegicus
MEDPGGAPLGERVPAPHPPQPHPLTAHSSSTPAPGWAGMQLQDPLPPHHTLAARSRQALPDPAASTLPCHPQSPRAGIGTPSAILTCPPVRSPPSPTAQSPAAMRQSGTSQPLLINMYLPDPVGDGLFKEGKSPSWGPLSPAVQKGSEQIQLWQFLLELLADRANAGCIAWEGGHGEFKLTDPDEVARRWGERKSKPNMNYDKLSRALRYYYDKNIMSKVHGKRYAYRFDFQGLAQACQPPPAHAHAAAAAAAAAAAAQDGALYKLPAGLAPLPFPGLSKLNLMAASAGVAPAGFSYWPGPNATAAAAATAALYPTPGLQPPPGPFGAVAAASHLGGHYH

SEQ ID NO: 4 - DNA, 1752 bp, Rattus norvegicus; identical to SEQ ID NO: 1

SEQ ID NO: 5 - DNA, 1752 bp, Rattus norvegicus; identical to SEQ ID NO: 1

SEQ ID NO: 6 - protein, 238 aa, Homo sapiens
MRQSGASQPLLINMYLPDPVGDGLFKDGKNPSWGPLSPAVQKGSGQIQLWQFLLELLADRANAGCIAWEGGHGEFKLTDPDEVARRWGERKSKPNMNYDKLSRALRYYYDKNIMSKVHGKRYAYRFDFQGLAQACQPPPAHAHAAAAAAAAAAAAQDGALYKLPAGLAPLPFPGLSKLNLMAASAGVAPAGFSYWPGPGPAATAAAATAALYPSPSLQPPPGPFGAVAAASHLGGHYH

SEQ ID NO: 7 - DNA, 1901 bp, Homo sapiens
ccgcggcgagtggagcgggagccgactggaagaagggctctagggagggggctgtggctgctggggtccgaggtggggccgggtacaccagccccatcactgtttgcagagagtcagggaggcggaaaagacacgcgctctaggctcccatcagggcacatggcccgggcccatcccccgcgcgtctccccggctgcggggcgcggggggctgccgggtgcgcttggctgtggcgcggcgcgttggagactttattgcgatgggacgataagaggggcgggggcggggtcctgggggccgaggcggcagcgctttaattaaaacggaaattgcggccccgggccgcgcgggggccggagggttccaagcggccccttagctggaagcgtttctccaggacccccccgcaacccccgccacgcccgggctgccccctcccgccaggccctgccggacccggcgccgtcttctcctccttgtcacccgcggtcgcttcgggcggggatcggtgccaccgagcgcaaagcctgcctcgccccccttccccgtcccccccatctcccaccgcccagtccccggcggcgatgagacagagcggcgcctcccagcccctgctgatcaacatgtacctgccagatcccgtcggagacggtctcttcaaggacgggaagaacccgagctgggggccgctgagccccgcggttcagaaaggcagcggacagatccagctgtggcagtttctgctggagctgctggctgaccgcgcgaacgccggctgcatcgcgtgggagggcggtcacggcgagttcaagctcacggacccggacgaggtggcgcggcggtggggcgagcgcaagagcaagcccaacatgaactacgacaagctgagccgcgccctgcgctactactacgacaagaacatcatgagcaaggtgcatggcaagcgctacgcctaccgcttcgacttccagggcctggcgcaggcctgccagccgccgcccgcgcacgctcatgccgccgccgcagctgctgccgccgccgcggccgcccaggacggcgcgctctacaagctgcccgccggcctcgccccgctgcccttccccggcctctccaaactcaacctcatggccgcctcggccggggtcgcgcccgccggcttctcctactggccgggcccgggccccgccgccaccgctgccgccgccaccgccgcgctctaccccagtcccagcttgcagcccccgcccgggcccttcggggccgtggccgcagcctcgcacttggggggccattaccactagacggggcggtcgggtgcctgcggcctcgcccgcacgcctagagtctcgcccgatcccatcggcatcccggggagggcccgggagcctccgtcaaccgtcctctaatccagagtttactccacctgccgcacttagcagggggacgggaccgaagctccctcaatccttgtctggtactagatttgctcctgtcccaccccgcagtcccctgaggagggcgatgtgcgccctctttcaccttttttcttctaggtctccaggtcccggaggggatttgtggacctctcttgtctccccaccactccagtgcatttccgcctggctcctagaagccccattcaatatcactactctttaacgagtgccaaatcttttcccacttttgctcttccccaaggaactgctcccacctcagcacgtggaggcctctcacggtcctccttcctgggacctgagcaggtttggtgaaagccaccgtcctccgtgacacacggcccccttcctcctgtccccacactcccaggagaaactcccggtgtgtttctgaccctttcagccccattaaagctcctgagctctcaaaaaaaaaaaaaaaaaaaa

SEQ ID NO: 8 - DNA, 1491 bp, Mus musculus
ttgtagctgggagcatcgctccaggaccccctccctccccgccacaccctggctgcccgctcctgccaggccttgccggacccggcggcgtctactcttccctgtcacccacagtcgccacgggcggggatcggcaccccaagcgcaaagctgacgtgccccccccccgtgccatcccccccatctcccaccgcccagtccccggcagcgatgagacagagcggcacctcccagcccctgctgatcaacatgtacctgccagatcccgtcggagatggtctttttaaggaagggaagagcccgagctgggggccgctgagccccgcggtacagaaaggcagcgggcagatccagttgtggcagtttctcctggagctgctggcagatcgcgcgaacgccggttgcatcgcgtgggagggcggccacggcgagttcaagctcaccgacccggacgaggtggcgcggcgctggggcgagcgcaagagcaagcctaacatgaactacgacaagctaagtcgcgcgctgcgctactactacgacaaaaacatcatgagcaaggtgcacggcaagcgctacgcctaccgctttgacttccagggcctggcgcaggcttgccagccaccacccgcgcacgcccacgccgctgcggccgccgccgccgccgctgcggccgcccaggacggcgccctctacaagctcccggccggcctggccccgctgcccttcccgggcctatccaaactcaacttgatggcagcttcggcgggcgttgcgcccgctggtttctcctactggcctggcccgaacgccaccgccgctgccgccgccaccgccgcgctctaccccaccccgggcttgcagccccctcccgggcccttcggcgcagtggcggcagcttcgcacttggggggtcattatcactagacggggacggccgggtacagcggggcctctctcacatggcccagtgaccaatcccatcctcatcctcggaggagccccgaagctttccccgacgttcctttaacacagatttcattgcagcagccgcgcccagcccagggaagaaaggatgggaaacccccgaggtcttccttgaacatcaggcttccaggccccgttatcttcgccccaggaagggtgcatgggctcccactttaatttttctcttccaagtctccagattctggaactcccgtgtctttttcttttctcacttgcacccctgcacctcgttatgacccctatttttctgggattttcttggtcctcattttctgactctctcccacaacccactccaaaaggtagtacctcaaggcttctcgcactcccctatttcctcgggatatgagaagcctcgataaacatctctgccgttccccatccctatatcaacactctggcttccctttctcccatatcacactctggcccaaggaccccagcctgtatatatccctttcagccccattaaagctcccaagcgcc

SEQ ID NO: 9 - protein, 237 aa, Mus musculus
MRQSGTSQPLLINMYLPDPVGDGLFKEGKSPSWGPLSPAVQKGSGQIQLWQFLLELLADRANAGCIAWEGGHGEFKLTDPDEVARRWGERKSKPNMNYDKLSRALRYYYDKNIMSKVHGKRYAYRPDFQGLAQACQPPPAHAHAAAAAAAAAAAAQDGALYKLPAGLAPLPFPGLSKLNLMAASAGVAPAGFSYWPGPNATAAAAATAALYPTPGLQPPPGPFGAVAAASHLGGHYH

SEQ ID NO: 10 - DNA, 41876 bp, Mus musculus
acgcgtggccagcctggataccacctgtccctgatccctcatgtggcttcctcagtatgatgcacaggccgcccaaatctgggactagaacctttccttcccctaacccctggctctacaggttaacgcccccttcaagatagaaatggccgaacacgtgctgaccaaagacccgagcttctcatgttccacaagccaaggcattgtgcccccgggggagaagaaatgcctgtcactgttctttcaccccaagactttggacagcagagccatcgactacttctccatcataccttctggctgtgccaccaaaactttgctccaggttgttggtttctgtagaggtaatggtcgtcccaggatgtcacgctccccagaactcgagtgtcccactagggagtcattcccttgagcgctttgtccactcctggtccctgggcctgggtgtctgctgtcccctggtggccattgtgggaaagcactgtggatacctggtgatcccatctttccatacagtaatgatggaactgagttttgcagggctccagggagcactccatccaagcggtgggacttcctatgagatatcttttgaggctggcaccctccagcccacccaaagggcaaggatctctcacagggacccctttcctctgcctaggtcccgatgcagtgctgcagcactcctgtgtcaacttccactgggtcaagctcggggaacgccgggaacagactctgtggattgagaaccagtcagactgccaggctcacttccagtttgatattgactgccaggagagtgtcttcagcatcaggcctgcctttggaaccctggcaggcaagactcgtatgaccctgcactgtgcctaccagcccactcaccctatcatcagtttccggagggtggcttgtctcatccatcaccaggtgagtgatggagaagggaccaagatgtggggaggaggccctgagactaggagatgaaggcaaggcttggtgggcaccctcctccttgcggaacctctaagggatctgactgggtcatctgcccctgtagagcctctgccatggtttagggacagagcatgaccagggtgcatgaaaagctcactggtccgggtgggtgtgccaagttctcccccactgccctcgggccattggctctgctgcttctgatgtcactgctgccagtcagtgacgtcacctcactcgggcatgcgcaggaggcgagacctctgaagctagaacccaagcccagcccccatgggcatgtgatgtacagtacctggacatggcaacaaaaatggaggctggaatgtagcctaggatcccccatcaactgggctctgggcttaacccctctacctggggaaaaggctcttttgcccggttctcataagtgcagcatgggctctgggaggccttggagcaaagagacaaagcagatgtggttttctgccattctacactggcagggtggggctaaatgccgggtatgataggcttctgtttgtcaccagtcagcaggagaatgcctgggaatcatgggtagtgcaagaagagcaagcaatagctgagcatcctagctacaggtccaggctgctctccccttgacttgccctgggataccacaactttctgctcagctagcaggcagctctcaggatgttgccctgaccaccatccttcagcctctgctccctaactctcctctgtccagccccccacttcctcttgcctcaccacctaccctgggttctgtcttgcatggacaaatgtcacaggacccactgtttctggacctgattgggacctgccattcagacagcatcaagccagccatcctgacacctcagcacctcacttggtaccgaacacacctggctcggggcctgaccctctaccctcctgacattctggctgccatgctaaaggagaaaaagctggaacgggacgaggatggggccctcatattgcccattgaggtatgtgagatgccagacatcccagccccacacaggcgggctgatctctccaggatccccgaccctcccttgcaagtcactgagtaggcccgtgccctacactactgtagctggggatgacccgcatggaacacttgaagcacttgagaggagggagccgaagagagtcagcagcaagaagaatgacatttgaaccctgtcccagtctctccctatgcaggaactggaggacctgccagaccaaaagtaccccaatatccctcccatgacggagtacttttttgacggcaccagggacctggccatctttcccccagctgtctgtctagagcccattgatgtggactttggtgcctgtcctgggcctgaagcccccaaccctgttcctttatgcctgaggaactataccaaaggcaagatcaccgtggtctggactggcagatctgactgccctttctgggtgaccccagtcactagtgacgtgcccccactcaagtccatagccctgcgcctgtacttccagccgtcctctcccaactgcctctatgctgtggagctagaagccttcgccgtttataaggtgtgtgcatacagactgaaacaactgtggaagctaagtctctccctagagcctgaagggcagagtctggccacaaggatgacccgtggctgaagggcatttcctaactgcttctattttttctttggctggacagtgccctcttctggttttatcctcccaggctcatggtctgacttggctctcctgagtctcacttctgtgtggtcttcccccttgcctccccccagggaagaactcagctgggatcaaaccctttggaaaaacttagtgcctagggaggagccctctggggggtgtcaggtaggggtccctggtcactaggccttaaagaaggtacccaaagcaaaaacattgtttttattttgtagtttgtagggtttttttgttttctgtatttgtttttgtttttgtttttgttttgttttgttttgcttttcaagacagggtttctttcactatacagccagggttggcctcaactccttgcaatctccttgcttcagcctcccatttctgagactacagatacaaactcacaacttgctgctcaaagcatggttgtggggttttggttttgttttgtttagagaagttcttgtctagtgtatgctagttttgaatttgttgcatagccaaggacaaacttgaactcctggtctttatgccttcatttcctgagtactgaaatcgcagggatggactgccaccctcaattaatttggtgctaaggtccaaacctagggcttgatgtatattaggcaagaactctaccaacttaactatactcttggtgttctctctctctctctctctctctctctctctcttcctcctccctccccctctttctttctcttctttctcttctttctttctttctttctttctttctttctttctttctttctttcttcccttcttgcttgctttcttgctttcctttctttcaagacagggtctcactgtgtagcctggaactccctacgtagaccatgatatatgccactatgtccactcagacatagattctactcagagtagaatctttgactgtcccattctcatatattagcagggctatgggagacaggtaggaatgtgatgctaacttctgctggccagttctgagtacagcagattgtgggttctagacctgtgctgatccataaggcaggcaggcaggcaccggccagtcatatgtgaccagtgagcacctgaaatacagtggtccaagtagagaaaatgtgctgtgagtataaaatacacccaaatttcaaaggctgactatggaaatagtgtaaaacatctcactaatagcctctccagtgattgcatgctgaagtgactggagtctggaactattgagtcaaataaaatagatgattgatatttcattctttaaagaatgtggccactgaaacatttaaaattgcattttcagccagagtctgtggctcatgttatatttccattggccagcactgttctgggttcattcctgtagaacaaacctgctttagctacctaccccactctcctccatacagaggttccagtaacagcagaagggattttgtacgtccgtcctaggcagtctccatcaaatcctcctttctaatgctcaatataggatacctccagcacagggctagggaggggcagggtgactgtcttctagggtcagtccctggactctgagtttcctcgtgagctgtcctctgcaggtccttcagtgctacagcaatattgaagaggactgcacagtggtcccgtcatggtgtctgaaggttcgggctcgaggtcacagctattcccctgccttagaacaccacatcccacactattccctggattcaccccaggtgagcacggccccaaccatcgccagagcaagatgagggagagaccccagcctgcgcttccttgttctctttctaatggcctcagtaatacatccccatgtcctttctggccatcctggggatttaggccagtgaatagcttgtttgtggatcatgtgtctccctgcacaagaacagcctagagtctgggtaccagccctaccctctccacctgggaactatactctccaacgccttcattggtcttgctcactgggtgcctgctggctcccaagcctaggctggactttggggatgaactagcccaaagcatgagccccgtcactcttttgagggtcagaactcccaaacctcttccctaggtccttgaaggtcttgtctgtcttgggaggtaggatagcacagggctaaagagtgcatggcctggggccaagcccccagaatccaattctgcttttccttcccaggttctaccactatggtcaagttttcatgttttgttgttgggaaggtatcactttagaacagtggcttgcacttccactctctcatcccaagtctatgctgccttgatatgcatcccaaggccacccctccttaaaataaagctccccctgacactctgagtgcctgcactaatgtccttgggcccttggcagctcccttctgtgtgaccagtggcagcctgtgctgcttctgcactgagtcacataaacttgttggtacaactggttgggaacttggaccaggggttataggccttgcgttgtgtagcctgtttagtttttagcctagttctggccaaatccaaaatggccaagaagtgtttagaagctgggactattggggctggccccctctgacatctgagtgacaatcatctagaaagatggaatgacgctagacttctgacccaggatggctgactccctaaagagtaactggaagactctgctaaaaactaacttcacctctaacctcaaggacagacctccagaggcctggcttttcctgggacctgggaccaccacatccctatgtctgaaggtctttgggggacagaagtattctttagtggtaggctgcggctgcccttaatctccaggaattctttgctggctgtgccgtatgcagagatcttggctctccttggagacagcatcatccaaacctgggtgggccccaaatgtgtatctagggacggagaactgaggaaggacgcctttccctcttattctccccacctcccagacgttcccagcagtgtctcctggaaaaccctcctacagaagcctgtttttggtcaacaaaggctccatgctcatgaccttcagcctggcccccaatagcagctcggacatcaccctgaggccctcctctggcctcatcggtcctggagcccaccaggtcttccttataagcacataccccaagggcacctcatggaggcagcacattttctacctgaacttcaacttttatccccagtacctcaaggtaggaggcaggggtagaggaactgggcctttactgggtcttcaggccaccacaatttgtcacttttttttcaaaaccacatttttagggtctgtgccaacatatggtgggatgggagtggggggcatttctttaatgaggaggaatgggagggagggagctctgggctccacccctggcgactcagctacttcccagtgagttgtctgctccgcggagttcagggctggaaaaaagctgcaaagatcccagacttatatctgaagggagtagaggggtgtttgcagtcagcccaggggcaggaatggatggttagacccatatctgaccccatgaaaaactgatacatcctttcttggtccctgagggttcaatgtttgtttgtgattttaagacaacaaatctaccaggcagtggtggcatactcagaagacagaggcaggtgatccctgtgagttcgaggccagcctggtctgggtagtgagtttcagagtagccagggctacacagggaaactcctgtcttgaaaaatcaaaccaaacaacaaaaaaaggcaatatatcactatatagcccagactgttcttagactctaggcaatcctcctgcctcacctactctaatttccaccacacctagctctgggttcttttgaatctgcttttgagagaagctgttatgttcagtctggaagacagctttggctgtctcctctcatgtgaaaatcagcacggatagttaaaaaaaaaaaaaaaaaaaaaaaaaaaaaccgaagggaaagccaagaggtcatggcattgtccttccttcctctggctcaggaagtgagcatgcagagcagagaggagcccctggatctgaagctggacacccacaaaagcatctacttcaagcctacctgggtgggctgttcctccaccagcaacttcacctttcacaacccctcacggctgcccctgcagtttgagtggagagtctcacaagagcaccaaaaggtgctggccgtccagccgtccaagggtaccatccaccctaacgagaacctcgtgagtgcctgggcccattgtcaaggagggtacctgtcacctttacctgtcttactagtcctgtggcctgtaacgcacacctgcctgagcaatctctagtcacctcccctagcctgcaagtgggctttagtcaaaccttccttacccacaacttacaggaaattgaggcttagggggaagaatattaaaaagatcaagaccagaggtgcagagttgggactggaggcaacattagtgatgccagtgcccagggcatgtctaaacttcatcattcatgttatccaagcaacaagattgctggcaggtagtttgagtcagctgggtatgggccaaatgggtgactctcttaggaaggggtgagaaaggagacacctggttccaggttaatacaaaagggggacccaaactgtcactaaaataacagatgctgtctcctattctgaggtaggtcagatgcagagcagtaactaccaaaacatggtatccatactttttttaaaaaaagatttatttatatttattatatgtaagtacactgtagctgtcttcagacacaccagaagagggagtcagatctcgttacagatggttgtgagccaccatgtggttgctgggatttgaactcaggaccttcggaagagcagttgggtgctcttacctgctgagccatctcaccagccccatggtatccatacttacccagggatgggaaataggagcctaggaaattcagatgtaggaaacaaacccacttgagtcctggggcccagcctctggacaccaggtttctgaccctggtccagatagttcacttttctgaggctccagtttccatcataatgagtctggaaaatgccaccatgtccctgtcatggagctgtaggatggaggggcttctggagatgagatgataggatgtgtgccaagtggcataagtaggcaagtgcaaacgtggtgcaagcatttgtaacaggacctcccccagccaggagtgggtgtcctggacacatgcatgcaagaatgagccgtctctgtgaccccagacactaacgtggatcttcagccctctggaggagatcaagtacctgttccgagtggggatctgggtctgggaagccagacagtcacaaaagaccaaaccccaagccactgtgcactacaggattcggctggtgggcatgggtgttactggatgcctctctgtgagtagaagccctgttgggggaggagccaggacccaggagggagggaaagttgtgcccagccctaagaaggcaggcaacaaggtagaggacaagatacggatccttgcaccccacctcagatctgcttctccttgggggatggttggaagattaggggttgggggcacccccagaagctagttggctgtgatctcaggcaaagccagtggagctggacttcgggaacgtactggtgaacagccaggaagtcaagcctttggtgctcttgaatgacggcaactgcaccctctattaccgcctggtcctggagcagcacaggcccaagggcctccacagtgacccctgtggtactgaggcctgggctggggctggggccagggaggggtgtgtgcatgtgtctgtctgtctgtctgtctgtctgtctgtctgtctgtctgtctgtgcttaagatagatggcccatacatactatgggtcaggcaagacagctgaaaaacctagcatttccacttttaaactgtggcaaatgctggtgacctcgagaggcacggctctttctgccccagcctgctctgccatcagggagtgctgtggtgacctggggaggagggtttacccgaggagggcacctctgtccagataccactgatgacccatgcattaccatctccctcacccccatcccagctctagaattcgaccactcggaagggaccatgccgcctcattcccaggataccatctacctgactgcccgtccaaaagtccgttcccaatattcctggaccattagctactgtctcctgtcccagagaggtccgtgggctctcggtatagagtcagtgccaggatggggagatgggaggaggtgaattgtgagatacagagccagaggcacgctaacttctagacaagctgacagtcagtgagagcagggtggcctgtgccttcaggatctgatgggtgtgactatgtctgtccacgtgatgtctacatacgtgtccctgagctgtggggacatgagagttagcgaatgtgctgtcatagtcacaggcaaatcctcagtgaagctttagctctcgcctccacgcgatggagtcgccgcctttctcccgtgccaatgttgggattgagtcggactctcccacacttaccggccgtgtgggactagctagctgtttcacctaaacatgcctcagcttctccaccaatgaaagggggatggtcacagactacaggccacacacacacacacacacacacacacacacacacacacacacacaaatgaatagcatatgccaccatagtgatggatattgaaagcaagacgctaagtgaaagaagccagtcagaaaaagccgagcaaccgagtgtctgaaacactcagaaaagataaaatacacaggtggagaatagattaatgatcgctggcagtgggggatcggtagatgggggagcgttaactagttagtgagttttggggagggagacaatggaaactttctaaaattagcattggtggtcccatgtgtctgtgaatatataaggcctattgaattgtgtgtgttaaggagcagtttttacagtctatgaatcatgttacattgcaatgagtttgaaaagagtgatgtaagttaaatgccaggatctacgttaagacaggacagtgcccagcacatagaaagtcttagacaagagctggctgccattggaaatatgctcaagattgaagctgagccactgtttgtgagcagggctctgcacctaaccccacgggagctgagctctctgctctcccaacagcccctcccaccaacagcatggatggcaaaaagaaagccctgtgccacgtgtccctggcagctgcatatcctttactctccgtcctggatatctgctccatgggtagcaccgagggcatcactcgcaagcacctgtggcacctcttctccctggacaccctcaatagttacttggcacgtgacccaactgccaaggagctcacctacaaggtgcccactcggcacaggtaagctaggtttggggccagggcaacacgggaggctcacaaaccacctactgacaccccaacaccaggcctttcccaagaagctgagtcgggcggggggagggtctctcaaaattcttagaggaagacaaggattgatgttatctcactcaccctgagttccgcatctttcactttgtgcctggggatgggcacccaaggttagagtcctgagttgtctccagctcctgcactgagccgtttctccacttcacacagcatgagccgtacaccccccatcttcactcctctgaagctggacttcaattttggagcagcgccacacaatgccctgccctcagtggtactcctcgtactgaagaactgtgggctggtgcccttggactggtatggctatgcagaaaggaggatgctacaggggtctggggacagagccctaaaggccaagagtcccagggaagagccatttcctgggtcttcctgggaaacccacatgtggtctccgggcaggtctcactgacagggtaagaaggctgggaggagactgtgattgtccacagcactcaggagatgtgattggctgcagcgaggctctgggcaggatgcaattcttggaacctggtcacagctatatcagacccaggatctgaatgctacagacccaggcactatgtttttctagctttcaatttttttaaaaggtttattggtgggatgggaagggaacctggagaaggggatagagacagagaaggagcgagaggagaaagaaagtggggagaaagagggaaagattccagccctgtcctgggacatggagggaagaggggaggatggagagaagtgggggcagctttcaattcttgatctgtacagtggtctggtaagaaagacctctcctgttaaaaagacagggcctaagggtcaagggactaagagcccaggcaaagccacatagctcacatggctgttagaagcagaggtaggttcaagtccaggtgtaccagttccagaaagtcttcctatactagtcagacagcaagaaggctttaagttgcttctgcatatcagggcggtctaggcaccacccctcgctgcttccgtacagattgcagtccaagggctggggtcaaatggtggcaagtgtaggttatcatgaagggccagatttctgctgccgcagaggatcctaccactggagggcgtgctgagcaaagaggctgcgctgctggagagtgagtggaagtcagccacaaaacagtcacctcacctcacaggtcacaggaagccaggtttaaggccctggacctgtcaagcctatccattgttctgttatcctgccctctctttggagcccagagcgcatgcccagcaagcggcccgtttccgtgccaggcttgttcgagttcccggtgtgaatacgggtagctttcttagtgaatgtctgcttacgaactttctgttctggttgactaatgagtataaaagaaggtagagagtgggccaaggtccaccctgactggtgggtgctgggaaagagagagagagagagagagagagagagagagagagagagagagagagagagagagagagagagagagaggagggagaggggaggggaggggggagaggggagaggagagggagaaagaggagaggagagtacaaggatataaggatacaagcaggggtatttgtggtgggaacttgctagcctgggcagatggaggacatttcaggagtatagggtgaagaaagggctctacgctgacaggtagacaaggttaccccaagctctgacatttctctgctgagttcataaagagtagatggaaccaggacaagtggtgtaaggagagttaggagttgggcttgagaaagcagtgggcaggatgaagggtaatcctgaatctgggaggtggatcagagagccatgaagctcccttctcccctcaacccttctcctcccgacacctctgctgggtttgaccctaggatatggttgcatagcaccttgcattcaaggggacagcctcaaagacatcaaatataccgttgaataccatttgttcactaatgtgctttttatttcattggctgcatttgtcccgggctctccccaaaagcaagctgggggttgatcctcagaatagaaatatggaccaggccaatccctgtcctcccgatgcaacaggaacccaggatagcgcagcctcctaggacaaggtagtatcctgggagggaggacagtgagacactcacagggtcgttgaccacttttggcaggataatggtgagatcagaaaagactttctggagggctggctgtgtggtgtttcagtactagagtgcttgcctggcatatgcagagccctgggttcaatccctaacatggttagaaggaaaaggaggaggaggaggaggaggaagagaaggaagaggaaaattttttgaagaacgccagaaaatggaggcctctaatacatctggaggtacaggcaagggcagggcacaaaaagcctagaatcttcctccagagagtttgaacttgttaagggacctatgacatttgagctgcagaggggacaaaggcccagttaacatccacgacctttcactctgatctccagggatggtggtccagaaaggcactattgtcctccagattatggggcaagcatgcacattagaacaagtcaggggacaggcagtgggatcatttcaccaggagaccagtcgggtaattgtgtgtaaaggagatgcagaaacttcccagaggatcctgaaatagcaaaagctcccacttggccactggccagcatttccccacatagcaggggagacacagcgctcacctgcaggaggccaatccccatgtagctgatgggggaagagcccaacccccacccagcctcaggctcatagccactgctggggtggccagagcttgggagtgtgccccactctcagcacttgcaccacacactgacagctggatgctttgcagagtgagtccctctaggatgtgtgccatgaaggacaaatgtagatagtagagtacatatagatctagtacattccttttatcagtcacatgatattctgcaatatcagttacaatagaaagggatttaagtgattaggccattcctttcttggaagattttgttcccaataaaatgaacaacaacaacaaaaaggtttttttgtttgtttgtttgtcaccacaaagaaagtgtagtatatggatgtatatacagatagtatagtgtagtatatggatgtatatacagatagtatagtgtagtatatggatgtatatacagatagtatagtgtagtatatggatgtatatacagatagtatagtgtagtatatggatgtatatacagataaaagtctgcagggctgaggagatggttcgatgggtgcagaagtttgtgtgacaatggctccctaggctttaaagcctggcaccattaggaggtgtggccttattggcatatgtgtggccttgctggaggaagggagtcactgtggaggtgacttcactttgaagtctcatatgctcaagctaggccatagtggggcagtcactagtgctgcctgcagatcttaggtgtagatctctcggctccttctccagcacgctgccatgcttctcaccgtgacgatagtgatgttttcctttttaagagttgtcgtagtcatggtgtctcttcccaggaatggaaaccctaactattcagtgggtgaaaggcttgatgcacaagcatgaggtcctgagcctggagtcccagaacctgttaaggccagccacactagcacatgtctgcagtcccaatgttgctgtggcaaagtgggaagtggagacagaacagtcattcccaggatctcgtgggccagctagtctgacagccacagtggtaaacaaaccccgcctcacacaagatggaaggcaagggctgacacccggagctgccctctgaccacatgcatgctgtgtcacctctgcagccgtgctcacatacatgtgcacatacatcacatacacacatacacaacaaggtttttttttttttttttttttttttttttagatgttcagcatgtgccggcatattttcatgactgcatgttaaccatacaggcagattcctgaaagtagggtttctgggccaaaggacacgtatgtgttttatcttagcagatgctgcccagttcttcccaaaagccagcaggacttccctctccctctgtgtgtgatattgtctgctctttccgaactttatgcatctatttttcaaatttcctgtgtgagaggaacaaggacagacccccctgtgagtttgaggtgttgttccccgactcccatgaggcttattggctcttttgccttccatttctgggtgtcccgtcttcggtccctgaccccggttgctgctgggaccagtccccccacccccgcccatctctttaagagggtgttctatcgaatgggggacttgggacccctcctctcttcttttagttttgcaagctgatgggggacaatagatacttttctacagcatgaggccttgtgagggagctcttcgcactcccacttgcttcaaggtcaccagccctctgggcacctgttctccaccctgtgttcaacaccctccttctcagggggccctgggtgatcctgccagccagcattcccaggatttgcaaatggccccctcctgagagcttatgaaagaaagatgagtggccagccctaagtgactcacacatacaagtgacacccttggctcctggtgtctaaaacgagagtcctagtcttctgttttctttattatttctgggtcttcatgtgtctttggttagtttttgtttgtttctttaaagacagttgtcttagtcattattcttttgctgtggtgaaacatcaagtccaaaacagctattataaaaggaagcccttaattgggtacttgattacagttttagaggattagtccatgatcatcatggcggggagcagatggacatgatggtgaagaagtagctaagagctcacctcctgacctgctgccagcaggcagggaaggagtgaaactgggcctggcctgagcttttgaaaccttaaaacccacctccagggacatacctcctccacatggccacgcccctccaacatggccacgcctcttaatccttcccaaaccattccactaactcaggatcaagcattcaaacatatgataggggactggagagatggctcagtggttaaaagcgctgactgcttttccaaaggtcatgagttcaaatcccagcaaccacatgatggctcacaaccatccataatgagatctgataccctcttctgggatgtctgaagacagctacagtgtacttacatataataaataaataaatcttcaaaaaaaaaaaaaaaaaccaaacctgccaggcatggtggtgcacgcctttaatcccagtactcgggaggcagaggcaggcagatttctgagtttgaggccagcctggtctacagagtgagttccaggactgccagggctatacagagaaaccctgtctgggggggggggggaacaaacaaacaaacaaaaacatatgaggggtcattctcattcaaacgaccacccagggtctcattgtttaaccctggctagtctggaactcactatgcagcccgagttgaccttcactttcagctgtcctcctgcccagccacccgagaactactattacaggtatgagccaccatgctcgctattcctaggtttggggacatttttgaaaggagaatgtctgtagctataattgctcaccagaacatgggtcaaagaacaggaggagtggtgaaggcccccctgtctgcctgtctttccatctaacctatcctttgcttggcttcactcctcttgtttgccttctccagggctttcctctttccaagtgaccagcagctggacctggatctgtgggtagagcaggaagacctcaacagcaatgagctccaccaaatgcgtgcagaggacaattccctcttctccatcaaccccaagactgggagcttgaaccctgggcaggagcagatggtagagttcacatacaggtgctcctgcgccccctaccatgcccttcctcccaagaggtctctagcccttgacttttctctgtatgactctgggataccctagacaatccccctccctggggatccatgtctcgcagggaaagtccatatcatttctgcctcattccaactcagaggtggttgtctgactcaaagccacacttgttttcccactccaagaagattccagaactcactgtctaacttgtctcctcccttgaagacacctgtttgtcggcaccgatcgcctttctgtgctcttcaaggtgtcccatggacgggaaatcctggtgagacctcacaccctagtcctgctggactaggacatccaagagagaagctaggagtggtgccccctaggactccggggcccgtatcaccgttgggtatgcccgttctcaaagggcagcacaccacccacggactctttatctacagctacagttcatcggcgtgacggtgaagctggagcagaagtatgtgcacttcacctctaccatccaccagttcattcctgtccccatcggggacacactgcccccacgccaggtcagtgctctgcgtaagcccgtcactgagaggcctaagtattgatccctgtgtgtctgtgtcagtcagacactgccccacatgggacaactgaggagatggaaggacaaagtcttctggtactaaagtccttttgccacactccattcccatgcagaagtactcccccattagcgcaaggttcttccaacttcttgaccaccagccacacttataatatattggatatcatttgtgcttcactctggtgtttatatatacactcaactagacttactgtgaatatgtacttaggctcttacatacaatgtacacagtatctcttttattttatcctgtcctgggttctacatgctagctatgaccctctcggttcatctagacctgtttgccaagaggtcttggccacagtttggaaatcattgcactcagagaagctcatcaggatgggcgtggaggacgggaatggggagctttagaagttgcacactcttctttcttttgtgtgtatgtgtgtgtacatgtttgtgtgtgtgtgtgtacataatcatgtacacatgtgtatatgtgtatatatgtgtgtatgcgtatgaatgtttgtgggtgggtaggtgtgtgcatgttggtgtgcacatgtgtatgtgtttgtgtatatacatatatgtacgtgtttgtgtgtatatttatgtacatgcttggttaggtcaagctggtcatcaactttttaaaaaaaaattatccttgagccgatctgtgagctccaggaagagactgattgaggagaatatacccatgttaatctggcctccacatgtatatgcaaacgtgtgtacatgcacacacatgtgtgagagtgtgtgtgagagagagggggtagagagagggttcttacagttctgaggactgggaggtccaagatgaagtccaggcaaagaccatgatttggtggtatctggtacacgcttgcactcctggctcatacaccatgtcttctccatgtgtgttcatgcagcagagaagacaaacgggtttccttaggcctttttatttggctttgacaattactggggttcacccctaatctgctttctaactgctggtctggctgccccccccccaacagagttcctcagatacttgtttctcctagccatgtgtatggaacattgccttccatggtcctccttctcctcctcccctctcctccccatctctctcctcccctctctcctctcctctctctcctcctcccctctctctcctcctctcttctcctctttctctcctcctctctcctccttttctctttcctcctctctcctcctcttccccacccccaaccagctaactcaaaatccacctacctctaactcctccagtaattggctgtagtcattttttgttttttactgaaattaagtttaaattaccttaaggaacaaggttcacacaacaaaagttggtaaactgtgaggactcactacaatccatagcaacaggccagacctccacagcatccatttgccactgatcaccacacagacacttattttccactctctgatcacttgtgactttctgaatgaaccatccactgtataaagaagtttctcagatgaggtttgagaactatactaatttatggttgtagagatgcaaagttagagggcagtttgatgtcatttagcagactaacaatagcaagttcacctctgggacgtgtaggatctgcaaccgtggattctagaccatgcatcaggcatacatttgctcttgtgaagaggggccttaaatctaatcagaaagaatttggttcgccctgggacattcaagtcattattacggctgtggacacatcgttgcctgctggttattactgtagtttgcagctcacagttgagcaagagaattgattcccccctccccccagcagcctacatcacaactccccatatgatgagagctagccagcaaggagagagcctccagttagtaccaacttgattgtattaggtttcaacctttgaaactgggtacagagtagatacagacattcaccctacagcaccagacctggagatcaatgtgatgggccaggccaaggaaaagggacggtccaggaccatccccaggggaattctgtgttcctctctggttttagtattcttagttctgcaaagaagtgtcagaccagagcagctcaggtttttttttccaagtcatcctcagttcttcagagcccttctataccctccttaaacagaatttattctaataacagatgagggacgtgacagcgtctccatggtgaactagattgaaagccttagatgacaaagggtctggatggtgtataggcatatgtggacatttccccaacccccagtgggaagggtacttaccctagggtcttttataaacttgacaagagcaaacaaagcaagctgctgtttgacagagttgctgactatcctaaagggttacaggctgaatctccatgttctgagccatagggaaaaaagattgggtggggccaggggctcgggcaccttcagatgtattgtggggataacttaccctcccaaaagaacagagtcctaggctatctgctctacaatcaaatatctccccatccctacccttgcagatttacgagctgtacaatggcggctcagtacctgtgacatatgaggtccaggtcagcgttctgtcaaaggttcaagaaaaaaattttgaccatcctattttctgctgcctgaaccccaaaggggacatccagccaggtactactgctcggatcctgtggatcttctcacccatcgaggccaagacctacacggtgagcagtggcctagatggtattgggggtggctggagttggagctgtgacttccttgctgccttccttgctgctttctgatctcaccagttcctttcccacctctgtgacttcccaggtggaggtacccatacacatcattggatggaactcagcggtcgtctgcttccaaggagtaggatatgacccctgtgtcatgggtgacaccgcccctttccacagcatctcttcgtgggacagcagttccatatcctctaggttgatggttcctgggcaggtgggagaaagggagcaggggaatccagaggcaatgaagggtcttagagtcaagggtggccagtgccaagggctggggaggctatggagcaaaggagataggttaagggggtagagggacgagtccttgaattagtctggctccagcttcaaagagcaccagctctgtaagaggcttgccctgtcacctaattcccagtataactgcttgggacactgaagtggaaatccacccatatcaggtgccttgatcccagctttttctgtgagtataggactgagggatattgcaagatttataaagaaatcatcctattctaggtactgtcttcatatagaacagtcctcttctgggttcaagtagattttgttcaagatatcagtggtcaccaatactacctttatagtccatacatttcttattgtcaggaggcctggtccattcagctagccatccatctgttcagctactcagatatgcactttcccataaacccactgaaccacacatcccttactaatctatgcattcatctatacagccatctaaccatggacccacgaacaatcactatactacatacttttccactagacccataattcaggatgaacaaatcacagaaggctttggggtgaggctttagggagctatcctagctctgactgaatgtgagaagaattcggaaacgttgcttgtgccccctgaaccttaacaacacacccaacacaagagaacaactagggctacagtagagggccaatgtgagggtcagatgtgtctaacataataattgtccagttggaaccagttgtgcttctatggcttcctcttcatgaccccaaagctgatgctatgttgtcttcaccaacagaatgtcttcctgtcccagtctcacatctccctgggaaacatccctgtgcagagcaagtgtagccgcctgttttttctcaacaacatctccaagaatgagacaattgtcttcacctggaagccaaggtccttagactttggagaggtgagagtccacatctgctcccagaatcagaacttagggctaggctccagcctcattctgtacagccccatctcaccccatcatatcctgtcccacccagcccagcctatcctagtacatcttgtcttgccctatcttacttcatactgtcctctccaatccaatcccatccttccctgtaccatcccatcccatcccatcccatcccatcccatcccatcccatcccatcccatcttggaccaccttgctgcatccttcctcccaaatccacaggctctttccctaaatcaccaggtaactgtgagtcccatggaaggagaagtgggtcctgaagagggagccccaatcctagtgaccctgaaggcctcggttcatgccagcttctacagcatagacttgatatgcaaggtaaggatgcactacagagaaacacacagggctgagacacagctcccaattaatggaacagagaaggattctgaggtctgcaaacccttagagaaaggcaggagccagtccttggaagggctcaaggtgagctacagctgagaccctccagacctaggccctctctcattttgtgctgggctcaggtataccagcgggaacttatgcggcagtatcacaaggagctacaggaatggaatgaagagaaggctcgacaggaggtggagttcaccatcacagatcggaaagtaaaggtgaggaaggctgagtggacctcacattcccagatcccactggccttaccagaggagatagaggtgatgcactggggtagctttccgtgttctgctcaccaccacctgccaacattcacagttttatccctcggttttgcctgcagccaccacgcaaaactctaaaaaaaaaaaaaaaaaaaagactctgcagcccctgatcggtatgatactgagctgccctcagaatgcaggctgctcccttgtctctctgatcccaaagacccagggtcccaaggggaagcagctttggggccagcctccctgtttgaccacattttcctctctatccttcacttggctgagcaggttttccttgtctcttcccaagtggcccttacctagggaaggttctagaagagagtagcactgggaggtgggcaaaaccaagaggggaacgcaaggtctggttatttagcgagatggaagtggacttggctggggctgtcacatcctaagctggactctctcttacagaggagagcatactgtgcagcccatgaacccccaaagaagtacaaggtgagccggaaagccccccagccattctggggataactgaaaaggttctcgattgtcctcagagatatccccaagccctgagccagcctcctagcagggagggaagaaggtaccccggggcaagtccacaaccctaaagagctgggccgggggacccactgacaatgggattatagctgccctaccacccatgcctccgtcttcccaaagacgctgcctcccatcacaaaccaaccgcctctcaaccggcctgctacctggaacctgaaattggcaaagaaggagacatcatggccatgccctcaaccccctgtgccaggcctgctgtgtctaggcctcaccgcaagggcccatgccacagactactacctggccaacttcttctctgagttcccctgccacttcctgtaccggtgaatatctggggaagcgtgggccaccttccttttgccttagtcctctctctctctctctctctctctctctctctctctctctctctctctctttctctctctctctctctctgtagagcagcagatgtccacactcagcctttaggaagggttgaagcaagaacagggctgatgagagtcattgggctctgtgggcccacatgatccccattactgctttggtcctttggaagagagtaatagagggacccaccaaggcttcccgctgtctccttttggcaacttggagcctctgggtcctgctctcaacttaagggttgggtgtggctctctgagtacagtaactatatcctgttcagactgtgagtgtgcctggcctatctatgtccttatgactgggcagctaccagcatgccttgtacccccattgctacagcctagtagttttccaagtccactcaggatatcaatggtctaccccagggagctgccaaagaagaaatcctccaaggaagagtcaaaatcttctgaagaactccctgacaaaaagggtcctgtctctcggcagaagcagcagcttctggtggactgcctcaccagcatcatccggtacccactgctccatgcaccttccttctcgacctctcatctctctagctacgctagcccaagcttcagaaggcctgacacttctccccccggtggggggggagggttcctttgcaagatcccttggctgggagacacttgggtcagaccaggtctactagagctagctaggtgggccttcagcctgaatcctggttctatcactttctgtggcaggcttgaagatgtctacaggagatctgccctttgtctgggtcattctggcttgctggctctgcctacatcccatcctgttcacctctaaaatgccacagggctcagagatgccttgttcctagggctgtactccctgacttagcacttgagagcaaaaacagtaaccagaactgtctcccatgggcaacagggagaccaaagttgccctggctagaagccctttcattagatttcatctttcttttctcctgcccaggggtctattggaagacaagaatttccataatgctgtggaccaaaacctggtggagcaggtgccttacttctgccagttctggaatgagcagtcagccaggttcctagcccagaaaagcagcttgtacttagtgccgatcctgtccctgccgcccagctatgagggcaggaaaagcaaggagcaggaggaggacctttttgggaagatgccaggggggcaagaggatgatgaagaggaagaggaagatgaggaggaggcagaagaggaagaggaggagatagaagaggagatgagcaaggacgaggaggacatagacaaggatgcaaagatgacctggtcagggattaaggtcacagagacctcccagcactctctgcagtggcaatggcagcaggatctgaagaccataataaaggaggagacagaaagtgacgagaaggaggccattgggaggtgagcagcgggggacccgaggaggactcgggagaagctgacactgaggctggcccaagctcttggtgtagtgtaagattcgggtgggaggattttcagaagagcagaacccagctttgtgtagtgtgtgatacctgattgatagcgtttgccagccagcactaccgggcatggagtggttgtgttcaaagtctctccagtggacaattaggaccagagaacaagtaccacttcttctgataccactgttttctggggcctctttgccgaggtggggtggggttgcattctgtccttttgggattccttgaccccacatagcttcttgtgcatcctctcctcctaccatcctccaaacccagctctgggattgcaactacctcacctaggtttgaacttagccacacaactagcatctgtgaaattgcaacgggatgtgcctcccccttcccccgcaccccccccaccgccacccctgccgccgagtgttgccaccagaagcagggctttccgtgggaaatctgtgcaaagcccactgcacagcacactcgagcactgctaggtgagaggtggcttgcaggtctgtaaccttgataacaagagcaatgccctcctcccaggctaccagcttttgccaacctgcaggaagcaatactggagaacatgattcagaacatcctggtagaggcaagccgtggagaggtagtgctcacttcgaggccccgcatcattgccctaccaccagtcagcatgcacaggtcagggggttgggcttagcctcattggttggaggaaggcaagccaggagaggcattcagatgtgattgaaagcctcaccctttctcttttgcaggacagacaacctcttgcagatgtcacaaggggacgtgctctgctctgggatgcagcatcctgactgtttgctggtgtcagcatccagcccctctaacatgactacctaaattgcccctggtcccttttttttcttactgttctgcaagtaaaccattaagttccacacgccaatctacctggcttttatttcctcctgcctggagaaagaagtatgttgagatatgtggaaggtgagagtttttgatgtggaccacagagacccatctatttagaaatgggagggtacatttcccgttgacaggcaagagttcttcttacttgcagtgaggtgttgttcttagtttggccagtggagggcgcttcaggagagacatagctgatctttgcaacagtgtaacttggttaacaaaacaaaaaacaacaacaaaaaaaaacccaaaaaacaaaaacaaaaacaaaacaaaacaaaaaagaggattcctaggcctggtgggctaaataggtagccatggagatagaatcttcccacatgtgttcaccagcacttccagggccaacctagttccctgaactatgctaagaggttagaatgaagctgggctgtggtggcgcaagcaaggaagcctttaatctcagcacttgggaggcagaggcaagcgtatctctgtgagtccagcctggtctacagatctagttgtaggacataatagctacaaaaggaaaccctgtctcaaaacgaacaaacaagtttaaaaaaaaaatgaggtacatcgcagaggccagatttatgatctgggtacttttcgaagacaaggtttggtgggctacggatgttactaaataacctccattgcaaaccactcctgagacccagagatgttctgccttcgctaggaggtccgcctctgttttctccctaagccctctgggcagcctctgtcttctgctcctccctgagctcagagccaggtgggtggaaaagtgacatctggtgttgttccagaggcgctcctggtcccggtaccctcccctgcacccctgtcccttccctccctctctcaccccccctacccgctgccaaggcctctgagcgagggggggggggtgtgctccgcctccatgagtcaatatttgccccgagcaaacgggcgcccggacaggtgtgtgtgtgtgtgtgtgtgtgtgtgtgtgtgtgtgtgtgtgtgtgcaggggcggggactgaactggcagtataagagtgctctgagtcctggctcgggcacaagcttcggggcgtgggctggggacgaagccaagcctggctggtttgggcggaggggcagaaacgctgtggcgggtggcccgggagggaggggccggccaggtgtgcacctgtggcgtccgccactgccgccgacgtgtcagccaccgcggtgctcaggtgagagcggcggctagcgggagcgctgtgcactcgaggaagctcatccaccagacaccgccgacgactgccgcccggccccgggtcttccgctccggccccgcgacgagcccctcgcacaaaccggacctgagcgttttgttcgttcggctcgcgtgaggcaggggcggcttctcagcatcagccttggggccggccagatcgccatgcaaacaccagccgccgtcgccaccgccaccatatgggtcgcagagactgagcacggtccccccaggggggagacaaccggttcccatcaggccccgcctgccctgccctcagactctgagctgccaggcatagctcgctgcagggtaatgcgggccgagcaggctccagcaggatggccgcaatgggctgaggtctggaacagatcccctcccgaagggggaaactgcgattctagtcctgctctgtcccagagaattctgtagccttgggggagtgttatcaaaaagtcttggttcccccatttgcaaagtcagccgcctttctcacaatagtcagggcctccactaacctctttattctgctctctggacccctagccactctgccgagtccctttaaccttttaacctcacagtgccagggttgtgcagtgtgggaaaaatctgggagaggtgaactgaggtcttctgggcagactcaaatccactccacctctcagactctttccactgccacctgaaattttcactttcacctcctctcgggtccgtgttccaatccaaccatatctgagatcagcagacagcaaaaaggacaaacagggtcttcaggcacaggaaagaactcaccaccttcctcaaaaccccctaatcttgtattctctgcagcccagtcagacgggctagactgggtctaggtagcacacagctcagccagagttcgtttggccatggatcactagccacaacctagatctggaatgggccacccccatctcaattactttaaatgctgtgacctggcttgcaggaaggggattttcttacactctggcagaggagctcaaaccaagagctccatacaagacccagcagggaggttaggccagggttgagtgaggctgtcctgtgattccaggtgccttccatgcccccctcatgacaatgtatggtgacaaaggctctcatacagcagcaaggagcccctggctggtttcccattaggataaagaagaaatgaaggagagcagtaacccacacaccaagacagaggcaatgccagggagacggtgtgataaataaaccaaattttcctagaactgctgtcttgtcccatcgcccacatgtgctctactacccaaccccctagttaccaggaggtcccactggatctgctgagcgcctgaggaatgtttgcaaagtggttctgatgaggaaagggtgggttcttgatggaactagtcccaaggctagactaggtccaggtgtcctcagatggagatgtgtagcccccatcctggcctccccgaccaggttctccctgtggaactgtcttttcccctttgcccttgctggcatcgagccatttagctggatctcctttcctgtgtctttgacagctcacctcccagttccccagacacagcgtggaggtagcacaccttgtggggttcagatcctgagtctgcacaactgaggtttgtccttggaatggtcccagaacctaccaaagcagccctttgaagtagggtggcaatagagggaccatgcagtgggctgagggtctggtctggtaacccttccttaagcctctttccccacatccccacccctaaccacttccaagattcacttttgcaggccatcaaggggcatctggcctctcttctgggtccccccccgcccccccccccccgtcaggctgaggtggagtctggaggcgcctggcaagcctgcaggtgctgggacagaaggggggtactgagcagctggtgtgcgggctgcccctcccccttcctgctccaagccagagcctgcttcctgtcttccctgtcagcactcctggtaaggggagggaacaggttgggggggggggagggaagagggaaaggacgatcatctaagtgctgccctgggggaagaggtgggggctgggggaccgaagaggagaaaaagccaaagggaaggctgagaaagagaggtcttagaacaggggccaaaggtagagagtgtagctcagagcctagtcaggtacagagcagaggggagccactctgggaagggcaggcagaggaagcaacagaacaggcagcaagacctaggggaactggaggcagagacaatcagagagagagagagagagagagagagagagagagatgggggtgaggggggggcgtcttgtgaaagaacaggagatgggagttagagatccagtacacagacgagtggtggagggagaagaaattggggtggggaaccgaccccaagggaggatagaggggggctacagatggcaaactccagtgaacactctcctcccgtaagaagccaggagatgggaaggagcagggctcacacctgcagtccctggacagaggctggtttgactccatctttcccgtcaggtcactgctgagtaactgccccgcccccacccactcgggagcatctggctcagccttggaacctctgggctcccgggaggcctctcagggtctgtgtgagaatggagagacaggggagtgggggctcacatccaggtgctaatccctgtgcagccagggtagtaccagggcctcagtgaccttcttggagattaatccccatggagacagggcagctcgccccctcactctctgcctaggccaggtgggtagctagccagaggcagggcaggtggggcaagccaggagcaggctggggagggaagagagccttcctcagaccatggtagggtcaaaggcaggcatggctgagaggccccctccaacaaccagcgtagtttacaggtttaaagaaaacaaacaaaaaacaaaaaaacaaaaaaacaaaaaaaaaaaaaaaacaaaaacaaataaaaccctgaggccccaagcagcggatggggtggggtgagggcaggcagaggccacttatgaagccatgaaaatcagaggaaccagaaggtggatcttcagccctacccctccatctttgtttgcttcttgacaccctaggcctgagcaagaaaaggggaactttgaaacacctcccctgaccctgtcaaacttgagtggcagagtggcttctgggagacagaggggccataagtgaaaaatggtctatgctaagtgctctcctgggaagccaggaggcaggatcaggttagaaaaggaggctgaggatcccagggttgtgcttcctgccttgaccccaccccatcccagaggaagtggggagaaccccccagacgaaggcatccataacctgaaaatataccaacagtaactgctaccatctgttgtgtggtgtgtatatcgcatgggtggttctgtgttttaaatacatttaaatcttcactggagtcaagacaatgattcccattttacagaggagaaaatgcaaagataaagcggagactcggccccacttttaaacagctgagctgggctccagagccctcctaacctccagtacctaaccgagtgatgccagggacggggggggggggggcatgtgtgcctgtcaacgctcatgagggtgtgctccagtgtgagccctccccagtgcttggcacatgatataagggactggacaaatgtctcttcaccaatgaggtgtctgttgagtcccgagacagggcagctaaccgatcaagttggtgggatctcgcccatgtacctgttaccgaggatgactctacccacacatggctgtggacctgtgtgcacgtgtcctctccctgacaacctagaagagtctagtagcggctgcccgcgtgtctaggcgagtgcacggtgaaagctgcctctcacctctctggatgggggagtctatctgttctccagctttactgagggacagagaagatggtgggataatgggtctatgggggtggggctcccccatctgcacaatgaactgagttatctctgtctcctgggagacaaggcttgtacagccccagggaaggagctcaccagcctgttctagacccggactagggtggggacaagaggaaccaggctgcacaggagaggctgcccgtgggactggggggggggggtgagggcacacacagaatttgcattttcagcccagaaggccctgggtaatttccctgtgaaatgaggggaaggggtgggatcagaggactctggggagaggaccttagccaccagcacctcagaactgcccgagagtacagagaagtacggctcttcatggagtgggactttatttaggggcggtgacttttcctgagatggagatgtgagcaggaactcagataggtgaaatgtgagagtcttacagaagcctagaggtttcttatggatctaagggttggtgtggaatgagtgttttcttacaatcatggggtggggtaggcgaagtagaatggggtggagatgcggataaggcctttggacttatggggtttgtatgtcagagatccccccatatgagatgattacctcagtgggggccacagtggaaactgttatgagatcctggtgaggtctccaggcgatttgggggtataggtctgaggttgctgcccctgccccgcctattccctgaatgtcaaatctgtggatgtggaggaggtgaaagggatggtactgagctggcaaacaaagccacctgggcatccatccttcaggaagctctaagctcaactgatctgggaaagcaaacaggaaccagctgctgggggtaggaggggggcatgaggggagaccaggggctggaattaaaccagcctggtggagaaaggaagctaatggaaagagtggatatccagcacagggaaagaatctcttagtcctgctctggtaagccagggtgtaggggctgcctgggggcctcttgtcctttgtccattattagtgtcttccctttcttggtctctgaggcttggggtggggtcaggagatatggtcctcagttgctgtaggtggaagcagtaggtgtaatggtagacaggtatagacagatatgccttttcactggttgcagatagaactaaaagccatggatgggtactgagacctatatattctcctgatgaggaaacgcaggtacagacagtgcgggaaagactggctgctgccacgaggagaatttccaactggaaagggtagccttctggtataaatcaccacccctaaacccaagtgacacctagtgttaaggagtcattttccctctacctgcagtgaacacacatctctgccgaatactggccaacacataaactggaagttgcccccgcacacctcaggctcagctcctgataaacatgacaaacacagccccacagtcaccaacagcacagatgtacagcctgttgtcaacaccacagcctcctctcccccccccccccccctctctctctctctctctctcacacacacacacacacacacactgcccacacactggctttctggttagaggagactggccccgccccctagtggccacctctataattgtcggatggagggagaagcagcgagaggtcttctgttttggtttgtttgtttgtttgttttgcctaatcttcctttctgtttcagccaggacttgtgaggcagaacagcagctagaatagtaagcatccaagattggctgttgccctgtatggagtctgggttttcccctatgttctggggaaaattacagctctttcatttttagctagctctctctaggatagacaagcggctaaaagctaattctggctcctccctcaatcaaacctcctcaggtttgccccattttccatccatgtgtgcctggcttgggttatagactattcaagtttgtaggtgtgaatctcaacagccatttctcaccagtgaatggagaggcctaagggaggagctgcctgccccttcccacctccagccccaaacttcagaaatggaattagtgggatgggaaaaccatcatccccaccccactggagactttcttatacacttcttctcggtcaacatttggcccacaattgaccatcttgttaatccaccccagctttatcgggttcctactctgtatccagcaccgtgctaggtttgaggatagctgggaacaaggcagacacagctcctacccttttgtgtagggcttacagtctagtagaggaggcaccagtgcattcatatactaggaggaagagatcttgagactgataaaggctaaagagaaaaacgtactcgatgggtggtgttttgtggctggtaccatctacagagtagcagaggagctagccacaccaagaatgtgtggttaaaacatcccagggaaatcgaagggcaaaagcaaaggctctcggccagccacggatgggtttagcagttggtagaaggaaggtgccagaggttaagcttccgggagttgaggtagggggtctggcaacagaacaaaaggcctcctggatgttcttaactatgaaagtggctaggtgtggatttgaagaaagccccacaccctgctgggcttcaatttttatgtttgtgtgtgcaaactgaacccaccaggctgctgagtctatagaggaggcacttggcaaacaggagtaacaaaatatttcccacccacctccttccctcttctgctgggttagcctctctcgttgttaaggatctgtaatcagcctgccttctaaatgacagctaaaagggcgaggagacctcctttggggtccctagccttttctgtcccatctccccacctccccactgctgcagattctggtgcctcggtggtccccgagagggtgggggaagactgagagccacttctatctaggacagttgaaccctctcttggcacttgtattcttaaaatccaggaaaaaaacaaaaacaaaaacaaaacccaaaaaaccaaaccaaaccaaaccaaaacccatgctagcgctcccgggtgcttttcagtgtttagtgccgcctggcggcgaggcctggtcttgacctcagtgccagagaagactccttggaaagctccgctcaggaagaaaagggtaacagcgaacaggtcctgagggccttggcgcagcgtccttagagaagggtcttctcttcctcctccctagtgtcccccaaaagcaagatcttcacctagagagttttgactttggaaagaactgtgctggccaggcttacgcagtcttagaaaaggctaaaccgtacgcttacggtcacgcctgagtgtctgtgccagggattgcttgttctgcccctaggctaggagtctctaccaggcttagaccacagtgaaagaaagcgcagagtcagcctgggctccggcagacacgatgtgcgcctcagcactttccgttgtcatgcatgcgggctgggaccagatcggtgactccgctgcctatgcagaccagagctggccaaagcctgccagctgactcagcttgcacaccccacccggattccacagggagaggcagccagacatgactataaagcagcgaacaccaggctcgcgcgcagcctctccattttccgtcggtgcggtgctcactcactcggtgagtactggggggagagggagtaacaagggaacctcccctcacgcccacccgctccgcacagcggcagcctgtgtccggatccagaccctagattcctcacaccctactggggatgggcaggcttaggctaagtagagtgttcccgagaagttagcccatgatcttgcccttcttatctgcagcgacatgagcagcgcccccgcgccgggctcggcgcccgtttgcctcacactctgggatgaggaggacttccagggtcgtcgctgccggctactgagcgactgtgccaacgtctgtgagcggggagccctgcgcagggtgcgctcagtcaaggtggaaaacggcgcgtgagtgatcctgctgtggctaatgggccaatttaggggttttctggcttctctcccttccaccctagccaaagtcagagcccggaacttcaaccttcactccagtgcgccgtagacagactcatcaaggagagaaatctttgtcctctccagtactcccaacccctccccaagacaaaggcaagaagcaatcgtggggccgccctgaaacctccacgttttctttgtgcaggtggctttgtggagacagatcctttgtgctctgcccatctgggtatcctagaagccaggttggggtcctgaagatagcagaggctcctttgccactatagcagtacagagaacggttggctgcctatgcagtcacataaaacccagaaggaagtcactggttcggcgatcttattaacctgaaacattccttctaaccgcgggcgataaacgggaaacctgggcctttctagctcaggtttggggctttcagtttccagaggagctgggtctatctgaggaatggtttagcgttggagaaatagtttgagtccccggatattttcaaccttagcaaacagggtgatgatgggggagagggagactgagcagatgtttcccagttcctcttctctttttttttttcagatgggtggccttcgagtaccccgacttccagggacagcagttcattctagagaagggagactatccttgctggagtgcctggagcggaagcagtggccaccacagcaaccaattgctgtccttcaggccagtgctctgtgcggtgagcacagccaactcctcccccccccccccccccacacacacacacgcgcgcgcgcgcacacacacacacaccccaacctctgatctcctgccaccaagctccaggtcccaaccaaagtgagttgctttgaaatacctacccacccaccaccaccccatgatgtatgaataaactcttgggtgggtggtagtggcatttgtggacctatccctgcatcagcaccacggacagcaccaaagaccagcgggttctgagcatgctggggtccttctgtttcaatccaccacaggtgtgtaaggatgtcacacagccctggaaggctgacatggctactgttggccctatgcacagtctcagaaaatgcagttcagaggcttatacttgtgtgtgtgtgtgtgtgtgtgtgtgtgtgtgtgtgtgtgtgtgtgtgtgtgtttgtgtgtgtgtgtgtctgtctgtctgtcccagaaagaaggtcctcactctacattggttctcagttaggtattggtgagtcctgggactgctggtgaatatcagaggacccagccctatccattttctcttttccaagaagctgtgaggggccaaaccccctaggaggaagactcttgtgttagagaggccagatgcaaccagactgccctacccacctgttctggctgctccatccagcaccccttagccgagcctcaatctgtgggaggcatcgattgggatctgaacaaggggttgagttggggagtcaatgagacccttagagaaatgtcttggttaatgggctcaacattttttttctcagaactcatggtttcctcctaaaaataggttcagaggaagttagtggtatttgcacctattttctatctactttggaggctgaggaagaaggatcacaaactgaaggccagcctagcatactcagggccctgggtttgatccccactaccacaaacaaaacagagactactacaataaaaaggttcctgtcacaggagtccctttagcattgacctcaaagaatctccaggaagtctctagtcttcacacctaactcagcaacttggtaggatccaggtagaatctctgtgcattccagctggccagacttactaggagccgatggggcaaactcctgaaatgcctgagaggcagcatgacgtgtgctgagcaggaagtgggtgactagggttaagactgtatccgtgaacaccacttaggagggcctgatctatccacaaacctcccccacccatttctgtgtctcagaaccacagcgacagccgcgtgacactgtttgagggggaaaacttccagggctgcaagtttgaactcagtgatgactacccatcactgccttccatgggctggaccagcaaagatgtgggttccctgaaagtcagctccggagcgtaagtcctggagctggaacagaggcgacaacatgacgtgtgggaagcctgtgagaataaaagagaagatggggcagtgagggaggatgctagaggctaacagagagactgggggggggggagtgaaaggatagaaagagaagaagcaatatagaccaggagccaggggagtatggtaggatgtggaggagtggggctgagtaaggagtgaatgaaaggtgcatgtagaaaccaggctcagacagattgcaggaagatacagagcaaaaggcagaggagctgaaacaatgtttctggagttggctgtccttagctggaaaagcagacactgacgggaccacagaggtgggagtgctgcaaagcctgctcactgcccttttctttgctggcctagatgggtggcctaccagtaccctggctaccgaggttaccagtatgtcttagaaagggaccggcacagcggggagtttcgaacctacagtgactttggcacacaggcccacactggacagctgcagtccattcgaagagtccagcattaggctccacattcccagtcatcaccagtaagattctcaataaaagctctagaatctacctgcccctcttgcctctttcttggtgtcttgctgtgtgtgtgcatgtgtgtgtgtgcacgtgtgtgttgaaatctagaactttgtgcttttgagctaagtaacctccattcttacacttttgaggattgttccctccaaccaagggctctcataaggccccagaaccttacctaacttagacgagcatgcatatacgcttggaggtatataggggaacagctagcaagagttgtgggatccaataacatggagccagttcctcatcttgccctgggaaagacccatcaatacagtacaaagcattgaaaacccagatgatgcctttaccatttgctagatagatcaacaaccctaccctaaagaagggtccaaagctgaattccagccaccatatactccctggttggtcctgcaactctcctctcccctctccctgccccacacaccatctgggccatcttcagtttgcatcttcatcctagcttccagccagccagtgtctttcaatagttccctttctttcccttcctggaaaagataaaagatagtgggaggttccactattcacatctacagaagagggttcgtccctctgagcacaatctcaagactataataccaatcatctgtatctcctatgatactaaactttgacatctacctgcaaactggagctattgggtctctctctctctctctctctctctctctctctctgtgtgtgtgtgtgtgtgtgtatgtgtgtgtgtgtgtgtatgtgtgtgtgtgtatgtgtgtgtgtatgtgtgtgtgtgtatgtgtgtgtatgtgtgtatgtgtgtgtgtatgtgtgtgtgtatgtgtgtgtgtgtatgtgtgtgtgtgtatgtgtgtgtgtatgtgtgtgtgtgtatgtatgtatgtgtgtgtgtatgtgtgtgtatgtgtgtgtgtatgtgtgtgtgtgtgtatgtatgtgtgtgtatgtgtgtgtatgtgtgtgtatgtgtgtgtgtgggtgtatgtgtgtgtgtgtgtgtgtgtgtgtgtgtgtgtgtgtgtgtgcaggttgagtatttaataagaacccctgagaatgagaatgtgtgagatgttgtgggaaagcattgagcaataggtacagggtcttctctcactgtgtataaattttacttaacttctgaactggctttagagtactgttgggtttgatgctccaactatcctccattcttcatctatcacattagcaaagaaattggggcttaaagggttcaccaatccaatcagtgactgtgctggggtttaaagatagggccccagataccaaatccaggtacagttaaggttaatttgagttgtattttaaaaactcagtcttcaacagagcctgaaatatatttggggttcctggttggtccatctcttgaaagatgatagtattatcaaaagctaaatgcagaacgtggtccttaggcacttaccatgtgccatcttattaagctcacatattcaaatgtttaagtattcaattttaagacaacaagactaggtacaaaggtgacttaacttcctcaaagtcacaggattaggaaatatgcaagactccagaacccagagctgaatctagaacccaggtcaacctggctatctcttgactacctgttgccataaagaggcaatgagcaatagagtgctggtatcttctctaggatgagtggcctctggaaaacatcaagaggttacctttagggatcaggtgtggtggcactcaagaggcagaggcagaaagatctctgtaagttcaaggccagcctggtccacaaagtgacttctagaatagccaggctacacagaaaccctgtctcaaaaaaaaaattaaaaatgggaaggaggaggaagaggaggagggggagaggaggaagaaaggaggaggaggagagaagaagaagaagaagaagaagaagaagaagaagaagaagaagaagaagaagaagaagaagaagaagaagaagaagaagagaagaagaaacctttaggaactgttagaaggaccttaagtagtttgctggtcttggtttgtttgctccagcttttgacagcttcttaagttttttctgggacccaggggtcccattctgctttccacatctatcatcctctggtagcttataagtacagattgaagtaatcatatctgagagtattaacctctggttctcctattctggacaggttggtggtctgtgaactcaggagaaaccatgacacacagcttggcagtgagacaatgggggggggggtggaactaccaaggactctctcactggcctttaacctgccatcaggaagagaaggaggaagtggagtggggagacttccctacacacatagacacacagagacacacacaaaggcacacagacacatgatatacacacacaaatgcatacacatacacacacaaatacatacatagagagagagagagagagagagagagagagagagagagagagagagagagagagagagaaatgggaccacatgaccacatgggtgggagtgagacagaacctattacctacccttttctttgctatcctaggtgggtggcctactagtaccctggctactattatgtcttagaacaggaaatgtgtgtgtgtgtgtgtgtgtgtgtgtgtgtgtgtgtgtgtgtgtgtgtgtgtgatgtcagtgcctcactccttcagcacccaagatccctgtcagactgagcacagagcctgagctggagatacttgcccatcatgcagctagtatttctaggtagagaccccctctgaactcttctccctgctttctcagcctccctctgagagggttagaggatgaacctctgtgcctcccagctcctcctactcttcccttcttgatgtttccttctgtcagcagtatttggaaggagcactaagacagcacatgaaaggaggactgagatcttgacagcacactaaggctccttcctgtgatgccctaagcttgttccctcccaaacacttcaccctctctgcctctgctcccatcagattctacatctgcatccaaatctccatcccagtttccataggattctgttgactgcatttcatcatcctcaccaactgcttgttccctttgactggcaaagatgaactcgccaatgatttacagccagtgagggtgcacagggtccctggagaacccgtgtaggatatttgcagcagcccctcatatgagcaccttaggaacaaggaccctgtaggaggcctgcccatttctagccccccccccacacactcctgtatttaaccaagagagaggacccagagaatcgcaggtctgacagcagctatcccggtcatatgaagtgtcacatggttgcagaaaaggcactcctgatgcaacttcagactcagatctgggaagtctgacaggcacccagataccctggtctacttgaaagccacatcatggattaggaagggcagttgtacaagacacaactctattgtgatcttggtaactctgcagtgtgattcagccctaaggagcacgggagtgtttgtgataatggggaaggatgatagagaaattgttgtggtgagaagtggagagatgaataggaatctacaggaaaaacacagcttgtattcagcatatattggctgtgagactggcaatctgtgtataagaattgagcaggttaaaaaagaaagtaaaataagaaggaaaggaagagaggagacaggacaagaaaaacagcaggcacaaagcagtaaggagacctagaaagaaggtgaggagaataaagaagagggcaattcagaagttgccaggggagatgccccagcgtcctgcctgaggcccggggccagacgagacaggaaaccaggaaatcgaggaggggatgggtctctagggacctaaagagagtaggaaaaaaggagggagaaggcacgggggtgggcaaagataaagggagccacggcagcgcggtagcgcggctgggagcgcagcgacaggcgagagggagggaagcggaaatttaaaagtgaagatgcagataaggcagcctggagacggggacccgggtagggggaggaaaggggcggagagcaggcagtgagagaggggagggagatggctagaaagagggctctggggaagggacttgaggctgtggcttcaggggtccaaattggggccagcattcccgccccaccgatgtttgctgagagtcggggaggtagaaaaagacgcacgtacacacacacactcctggccccttctgagctctgaacaggagcccatcccccgcgtgtctcctgggctgcggggcgcggggggctgctgggtgcgcttggctgaggcgcagcgcgttggagactttattgcgatgggaagataagaggggcgggggcggggtctggggggccgaggcggccgc

SEQ ID NO: 11 - DNA, 45639 bp, Homo sapiens
  misc_feature (636)..(728): n is a, c, g, or t
tcccccaagcccagaaggctttgccttttgtcttccatgatgttacaggctaaattttccctggggccacctgccttcttcgaggggctgttcagctggggccacagaggccctggtgggttcccccaagactcccccatctctgccccagccaagtgcgcccagctgctggtgagcataaagcacaagtgcccggaggaccaggatgccgagggcttccagaagctgttgtactttggctctgttgctgtgggctgcacctcggagaggcagatcaggctacacaacccgtcggcggtaggcaccaggcaggaggggcggcaggactcacgcccccaggattggaatttcctaaaatcaccgccttgcagaatgccccccctttctctccatgcctttggccccaagcctcatgtaacccatgtcagctgtcaacaggtgtgtccttctcctcagctgggggctgggctggggtataccctggctccccagagccacgagcagccctaagactgtcctgaccccaggaccacagttagctgtgatggtaccctggggagaattaaaaggtgccccctttgggcaggcccagcgcagggcacatgactcggtcccttctctcctcctggtggaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgctctgctctgtgacctgaccctttgtccttctgcttggccacacaggtaaatgcccccttcaggattgaaatttccccggatgaactggccgaagaccaggccttctcatgccccacggcccatggcatcgtgcttccgggagagaagaaatgtgtgtcggtgttcttccaccccaagactctggacaccagaactgtggactactgctccatcatgccttctggctgtgcctccaagaccctgcttaaagtcgttggtttctgtagaggtaccggtgaccccaggggcctcccctggggacaggagcagcccctgctgaggattctgaagagctccttccctgctcctaggaggcggggccaccttgcctcagcctgggtgtctgctgccccctggtggccctgtggggaatcatccgtgactacctggagatcccagtcccactggcatccagtgatgctggagttctgctcaggctgccagggctctgggcctgtagcctggccctgagagtaccacctcccttcagtggaactttgtctaagatatccttggggaagctgataccccatctcctgtgtccaggccctgctgtgtccctgcagcactactgtgtcaacttcagctgggtcaaccttggggagcgctccgagcagcccctgtggattgagaaccaatcggactgcacggcccacttccagtttgccatcgactgcttggagagtgtctttaccatcaggcctgcctttgggacgctggtgggcaaggcccgtatgaccctgcactgtgccttccagcccactcaccccatcatctgctttcggcgtgtggcctgtctcatccaccaccaggtgagtcgtggggagggcatgaggctgagggaggaggggctcccctggggcccactgatgatgacaaggcttggtgggcacttccctgtattctggaacttctagggctgtctgcctggtggtctcccactcctgggccccagccctggcttttggagccagtgtgcagcctgggcacagggcagatcactctgtgccagtcttgccctgctaccaggccaggcgctttgctgctgctgtgctcatgggctgggagagggaggcttggactcgcagcctcccaccctcccgctgagccctgcaggctttgccttttctttgccttccatgacttcacagactaattcttccctggagccaccccacatagcatgcatagggcagagagctttcaagttagaccccaaccctaggtccctgggcatgatcagacacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaaatggaggaaagaacagaagcccagagggaggcctgatgaggcagccccccatattctcctcccttgggagggcctgaattctagtttggcaggcctctgggctggcagtagggtgggtgggagaatgcccaggggccatggcagagaggaagggtgggtaatggctgagtgtcctaaggaccggggtgctctctcctgaccttccctggactcctgctgtgccctggctgagccagaccactgctgagtgcttgctcactggccacctggagcttctgccccactgcctctgttatcctgtctgcccagcttctgtcctcagttcaacccccagcaccccccttgccctaccacccctcccatgccccaccacacacagacaaatgtcacaggacccactgttcctggacctgatggggacctgccactcggacagcaccaagccagccatcctgaagcctcagcacctcacctggtaccgcacacacctggcccggggcctgacgctctacccccctgacatcctggatgccatgctgaaggagaagaagctggcacaggaccagaacggggctctcatgattcccatccaggtacagtgccctccccagctgcatatcccttctctagtgattcttgggcaatcccatgcccccagggtcccatgggcaccattagtagtcagggcgactggtgtagcctttctagacaggagtgtacccaggaggggcaacagcaagaggaatggcagttgagccctatccacctgtctttccccatgcaggatctggaggacatgccggccccgcagtacccttatatcccccccatgaccgagttcttcttcgacggcaccagcgacataaccatcttccccccgcccatcagtgtagagcctgtcgaggtagacttcggtgcctgcccagggcctgaggcccccaaccctgtacccctgtgcctgatgaaccacaccaagggcaagatcatggtggtctggacgcgaaggtctgactgccccttctgggtgactccagagagctgcgacgtgcccccactcaagtccatggccatgcgcctgcacttccagccgcctcaccccaactgcctttacacggtggagctcgaagccttcgccatctataaggtgtgtgcacgcaatgagagggaggaatgcggggtctctgctaggagcctgagtggcttggtggggtggcaggaagtgaccgagggcagcttcaggctccatcctctgcgtgccaggctttctcttggctggacagtgacccctatgagtttgtctcctccaaagctcctggcctagctcggcccatctgattttctcattcttatgtaagtctcccctcccctccaagggagaactcagctgagatcaagctgtttgggaaaactgggtgcacagggagatactccctggggctcctggctaggaggcctcctagcttctctactagtccttgaattaagaagtggtcactctaaaggagctttgagcgggcaggaagctgggcctagagacaaagtcagcagcaccagataattgtgatggaaagggcttctgactcagcttccctgggtcgggaactccgagtgccggctgtccccagccctgctgttctggccccagatgcgtggtgccccctcctcatccgttagtcctgcccatcccttccttcttgactctgcccaccccactgcccttgcccagaggccaaggtcttggggcccagagaaaagtagggctgtgcggtcaagatcaggnnnnnnnnnnnnnnnnnnnnnnnnnnnnnnnnnnnnnnnnnnnnnnnnnnnnnnnnnnnnnnnnnnnnnnnnnnnnnnccacgtccgatcttttgaggaggctttccaaagtggaggctttggttgcccccgtcctaatgctctggcagtggggtgatgttgaggtttgtaggaataaggttgtagatgcctggctctgctgaggttcagcctgtcagatatttaggttacaggctctagacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctctaggctcattcttacagaatgaatggtgctttcacatcccagccccaccctcctcccgggttgtgaggactaaagagagcattgctatatggatgcagggcctagctccccgcaggaggggctgggttgggattcttgttctctgcactgtcttcccagggaggccctcctgtctgtgtccaatgtcagactcctgcagcacaagggcaaggaggggcaaaatggggtgccccacagggcagacacaggactctgagcttttccgtgggtctggcccctctgcaggtcctgcagagctacagtaatattgaggaggactgcaccatgtgcccatcctggtgcctgacggtgcgggcacgaggccacagctatttcgctggctttgagcaccacatcccccagtattccctagatgtccccaaggtaagcgtggccccaacagcagtgggagggagacaggggagccctagcttctgccttggcctgcagatccctctcccattctctgatggcctcagaaacacatctttgcaccctccctggctgtccccacgtccgctgtgccaattctgggtatctgggccggctgaggacttgctcctgggccccgacactgggactggggagagtctggatcctggacagctggccccttgctgctccattggaaatactcactggctcctatgactctcaggttcaaaaccctccagctcctgcatttgtttgtgtcctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngccaccccaaggcccttagaagccctgcctgacctgacaagcaagaggannnnnnnnnnnnnnnnnnnnnnnnnnnnnnnnnnnnnnnnnnnnnnnnnnnagtttcacctttgatgtctcctccagtctccatcgcctggaggttttgcattagacttctggttgctggaaaagcaccactttgggacaatgtgctttgggttgattctgttccccctaccacctacacttctgccctcctatcccaaacatataccaccccaagattcagcttagaggcagctgcttcccaaaggccttcctgcctcacagagtccccctcgatgtctccctcggcccccttcccttggcagttcctcctggtgggttgtggcctttatgcagcccagaagatctggcgtcactccctcaccggctaacacacaggcactgtcaccccagcttgatggggacctctggtcagggattgcaggttttgtgttttgcagcctgctcagctctcagctggtgctggccacaccctggatacctgaagagtgttgggagctgagattatctgggccagtttccatcagttacctgcatgagaatcatctagaaggggttacttacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgaagtggttacttactgcagccccagagcttctggaccaggctctctgaaaatgagttctccaaagagtgatgcaaagacaccctaaaatgaaacaacttctactttatccttgaggaacggccttggaggcctgagtttcccaggggcctgtnnnnnnnnnnnnnnnnnnnnnnnnnnnnnntttctggaggtctctggagaacaaaagcattctttggtgtcagctcgtggcctccttcaattccccgttcccagggcgttgactgggctgcagggcagcacctctgatctacttggagacagctcctcccaagcccccatagggccctgagtgtgtgtcccagggtggaggtctgagcggggggccgctttcctcctctcccacccccagctatttccagcagtgtcctccggtgagcccacctaccgcagcctgctcctggtcaacaaagactgcaagctgctgaccttcagcctggccccccagagaggctcagacgtcatccttcggcccacttcgggccttgtggcacccggggcccaccagatcatcctcatctgcacctaccctgagggcagctcctggaagcagcacactttctatctgcagtgcaatgcttccccccagtatctcaaggtaggaggcagagggaggggacctgggtccttgggaatgggccataatcttgaaccacccttttcaaggccatattctggggttggcccctaagatttccagggggccacttctcctccaggaagtagcatagtagggaaagaacctgcttgaccacttctgtgaacaactctcccacccctcagggccttgcctgtcccccttatggggtctgaggccaagaaagagggtgcagagaacccagaggaaggatctgggggaggcaaaagacacctgcaggtggtagagggatatttgggtccaggccagcaatagactctgagatggtgggcccatgtctggccccgtggaatggtctatagatcctctctcagtccctgagttcttttcaagaattatctcctgggccagtcaccattgactctgcttttgacagaaactgccggtacacgggctggcagagggcctttgactgcctgcttcaagtggggaaagctgcctagccatggcttcctggacatgttgtggggggctgggactgcagagtgaggtcagggcactgcccttcttcctctggctccaggaggtgagcatgtacagccgggaggagccactgcagctgaagctggacacccacaaaagcctctacttcaagcccacctgggtgggctgctcctccaccagccccttcaccttccgcaacccctcgcgtctgcccctgcagttcgagtggagggtctctgagcagcatcgaaagctgctggctgtccagccctccagggggctaatccagcccaacgagagacttgtgagtggctcccctcctgccgagtgacggcaaccttcacttctgtcatctcttatttgtgcctatcatagcccagacttgccatgtgcagcccccaattccatccacagtaacttcccccacctggcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgagaggcatgtcacctccccatccatggtccccaaagctgggaggtcattgggagggtgggttgcagcagctggatttgggcccaaatggccagccaccagtgagggtgaggaaggagactgtagtcacagtgggggtgctggctgggattcaaaatagcagacccctgtccctagtctagaaccttaccgacgggttggggaggccaaacttgggacaagaagcaccagcacaggcatctgtaagagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtagcatgacgccctgccatggggcctcggatggccaggtacctgtggaggggcttccgaaggccactgcagggatcatttctgtgaccccagacgctgacgtggaccttcagccctttggaggagaccaagtacctgttccaagtggggatgtgggtctgggaagccggcctgtccccaaatgccaaccccgctgccaccacccactacatgctccggctggtgggcgttgggctcaccagcagcctctctgtgagtgggagggctctggcaggagggggctgggggagaggcccggacaggtgggagggaaggccccagggaacctcccaaggtgggagggaaggccaagagggcagaggataggagccctctgccccggataccccaaacttgcccttccttgggggatgctctgaaaatggggctgggagagtggggctgtgggcctggaggagctcccactcccggctggctgactgtggcctccaggcaaaggaaaaggagctggcctttgggaatgtgctggtgaacagcaagcagtccaggttccttgtcctcctgaatgacggcaactgcaccctctattaccgcctctacctggagcagggcagccctgaggccgttgacaaccaccccctcggtacttgggcctgagctgggactggggccaggggaggtgggaggaggtactggttaagaccggttaacctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgtggtccctctgacccaggctgggaggaggggctccactccctgtttcctgcctcagggcctgctgtcctcccctggtgcccagggaagggtatctacaaaggagcaggaggaatggctgggcctcctctacccactgaccctccggcatcatccctgagcagctctgcagctggaccgaacagaggggagcatgccaccccggtcccaggacaccatctgcctgactgcctgtcccaagcagcggtcccagtactcctggaccatcacctactctctcctttcccacagaggtacctagactgcctggcatagagagtcggggccggggaaatgcaggatgaagggggagggctgtgtgaggcagtggatgccgagcgtcaggcagagctggcagggtgagtggggtgtgctgaggtgtgcctttggtgtctggcataaatgccacacaccagacatgtctgtccagatgagtgtccatatgtgcatgtgcccagtgcaaagagttaggagctgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtccagggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggggatcataatgtcacacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttagtttgaaaccaaatcacggtacacnnnnnnnnnnnnnnnnnnnnnnnnnnnnnnnnnnnnnnnnnnnnnnnnnnnnnnnnnnnnnnnnnnnnnnnnnngttgttggaaatgccctagagcccgagaggggtccactgctcctgatgagggtcctggggccaactgcccaggactcctgcacctttggcctgcatgccccctccagggaaggtgggaggcaagcccacttctgtcttcctcacccccctccccagataacaaggctggggagaagcaggagctgtgctgcgtctccctggtggccgtgtaccccttgctttccatcctggatgtcagctccatgggcagtgctgagggtatcacccggaagcacctgtggcgcctcttctctctggacctgcttaacagttacttggagcgtgaccccaccccctgtgagctcacctacaaggtgcccacccggcacaggtgagctgagcctggagtgggggtgggggctgggggcagggcctatctagggatgttcatggagctttctgcccagactcccagaacaaggaagctgtttcccatctctttgagggaagagctccccagttctcgcccatccctggaggggcccagggaagggaagaattggcttgaggtctgaagagccagcccacccttgcgcctgagtcccagattctccagttcccacttggagggaggggagtgcccaagccagccatccctgggagggccctgccctggccccatccccatctctgaacccctttctctgttcctcacagcatgagccagatcccccccgtcctcacccctttaaggcttgacttcaatttcggggccgcaccattcaaggccccaccttccgtggtattcctggccctgaagaacagcggagtggtgtccctggactggtatggcttgaagggggccactggggggcctgggagacaagtgcctagaggctgaaggagaacaagagctgcactctcttgcccgtggtggcctggctgcttgctgtctgtgaagagtggacctcacattgacggctgtggaggccagggtggaactggggttgccagcaggactccgggcctgtcaccggctgagacacggtgataggctcagaagcggtgggactgccctgatggagcctgggcatagtggccttaataacttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcaagaagactgagttgcttctacatagtacgcactgcctcgttgctggagcccgcctgacggagccccagcttagtggaggctgggcctgaatgggggcaagttgagggtctctggtggggaaggacagggacctgctgccggggacagagctgcccccgagggtcctggctgaggggcttttctcccccagaggctcagaacttgtgcccagcaaacatcctgcttccatgcatggacttttcctccctccaccatgtgcctttgtttcctagtaaactccttttgatgacttccaactttgcttggctaaagagaggtggaaaaaaaagtgtccccatctgggtcagacttaggagagggatccaggggtctggccagggcatggcagtgagtgggggagaatgaaaatatgcaggtgggggctcctgtgatcaggaactcgctggcctgagcagagagagccttggggaatgtggagtgagaggggtggggtctgcatcatcagatgggcactagcagcccctcactctctgactgtcccctttctcctccatggtggaggaagatggggaaactggctctttagggggctcctgtctgagaagggccagaactgtgagtaactgagggcttctagctctgaatgactgggaaccttggcagagctgccagcctggtggggaaggccagggcctgccttgagctccagacctgccattcactgggctctttgagtgggcccggagccaggccctacagggctccagggagagggttgggggcagaaaaagcggtgttggcctgtgtcctggcatggactgcgcgctggtgtttgggtaggagcaaccagctcacactctctgtggagagggtccaggctgctggaaacagaggcgggggaaacccaggcagcatgcccggccagccgggcccatccatcaggagatcatctgaggtgcaggaaggcagtccgtgcctcttccaggattctaagcagattgactctcctatgcttttttgttgttgtatttcctttcaatttcttagaaggctatttttgaaaattctagtccgtctcttctgagctcataaagaacagacggagggttttattcctgccaaggcctgtcagagagggccaggaggcagcagcaggcaggaaccccaagtgggagggggagctccaggggaagcagggaaccatgcagctgccccactccctccacgccttctccccttcgcgcctctgctttacctggccctggccttcgtagggtcttgcatttaaggggagattcttttcagatgtcaaatatgctggttaacatcatttgttcattcatccagtctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctcctgttgcggggtcatatgtgctgtgaaggaggtgtgtgcagcatttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagggagactggaccagggcagaaggcagtgaggacaaaggagaggctgaaatattaaaggaccccaaactagcaaggtctccaattttgccactgctgaatatccacaataaaatgacaacagtggtttactcacttgtaacaagtcagacaacataggagctatgggaaaaaagtaagaagctccgcagccccctctccaagtccagctgctagggtgatgaggattcggaatgtgcctttcacactcagcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctattcttcatgcttcagatgtgaaaaaagttaagactggttccttctgagggtttggacttaggcctccctgactcctactgaggcttccaggcccgtgttgcacgctttctctcaggtgtgcgttgtgtgctttctctcaggtgtgtgttgcgtgccttctttcaggtgcgtgttgcacacttcccgtcaggtgtgtgctgcgtgcattctcctcaggtgtgtgttgcacgcgttctccttaggtgtgtgttgcgcacattctcctcaggtgtgtgttgtgcacttcctctcaagtgtgtgttgtgtgcattctcctcaggtgtgtgttgtgtgctttctctcaggtgtgtgttgcacgcgttctcctcaggtgtgtgttgcacgcattctcctcaggtgtgtgttgcgtgcattctcctcaggtgtgtgttgcacgcttcctctcaggtgtgtgttgtgcgcattctcctcgggtgtgtgttgtgcattttctctcaggttgcttttgtgcgctttctctcaggtgtgtgttgcatgcattctcctcaggtgtgtgttgcgtgcttcctctcaggtgtgtgttgcatgcattctcctcaggtgtgtgttgcgtgctctcaggtatgcgttgcacacttcctctcaggtgtttgttgcgcgcattctcttcgggtgtgtgttgcgcactttcaggtgtgtgttgcgtgctttctctcaggtgtgtgttgcgtgctttctctcaggtgcgcgttgcgtgctttctctcaggtgtgcgttgtgcgctttctctcaggtgtgtgttgggcactttcttttccttttctggatgctccctatcgtatcctccacccggtttgcagctggaaccagttctgtgccatctccgagagtgaaatgggttctcctttcagattgtgacctcaggaccctttgtcactagttttgtcggctacttaaggtgcattatctactttaccgcagtcctaggcatgtcaggggcattaactctgtgcacagccttgcttctgtttcaagagtgccagccctgctggggcatctgttctccaccctgtgcccaagttggcctggacaccccactccctggggctgctggttggtgctgccagccttcctaggtgtcccaaatggccctttcccaagagctttgtcacagaagggttcatggccagctcaaagtggcccccatattcaagcaacaccttgactcctggcatccacagtgggagccccactcttcacttcttgagtcgccgtggcccagagtctgttcctgggttttggggccattatgaaacgagaatccacaacagccacatttgctcaggcacacatgggacagtttgccaaatgggtgcatgaagtgcgggagtcatggaagaagcctccacctgcccatcagggtctcagagcccctgcctctctgttctgcctgaccagtttctttgtatggctttgcacccctctcccagggccttcctccttccaagtgaccagcggattgacgtggagctctgggcagagcaagcagagttgaattccactgagctccaccagatgcgcgtgcaggacaattgcctcttctccatcagccccaaggctgggagcctgagtcctgggcaggagcagatggtggagttaaaatacaggtgttccccaccccccaaaccccctgcacttccagtcatagaggcccctaccccgggcatttcattgtatgactccagggcaccccaaatggccattccctgggaggaccaccttctcacctgcagaggaggtctaatttccttcttgccccatcccaactcaagggcgagttgctcccaggggctggccagctcaaagccacagaaatgtcatgtccatttgctctcctacttgtaccagggctccagaactcactcccagacttatctgtttctttctagccacctgttcatcggtactgatcacctcccagtgctcttcaaggtgtcccatggccgggagatcctggtgaggccccagcacactggccctgccctagtttcaggctggaggagggggctggctggacaggagcagggagctgtgttctcctggggaactggggtccagatctccactggctgtgtcagttcctccgggggaacttttatttccagctaaatttcataggtgtgacagtgaagccggagcagaagtatgtgcacttcacctctactacccaccagttcatccccattcccattggtgacacgctacccccacggcaggtcagtggtccacattgtcctgttgaccaagaaggcctaagtgttgatctctaggtatgagtgtcagcccttgagagggtgacaaagacatggccctctggaagcattgaggggctgggccagcaggttgaacccccagactgggcaactgggagagacttgaagggagaggctgcagtgtcctagttcctggtgctaagccctttgccaggcccagttccaattaaaaaatgaggatttttttttcctttagcaaaatgttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctccaaaaaggatgtccttctggaatgtgaagaactagaatgtgaattcttgggaagctttagacctttctttctttactgattgaaggcaatacagtttccttgctctggaaatgtagattggtaatctccaagagggggacataatattttcccaaacatttcacagctgtgcaatttctaatcatttagaagcccatgtgtttctgagcaggggcctccagccattcaccctagaggccaaggaatgggcttcctggatctagaggggagtttcctatccccaactctgttcacttccatacccacccgctacccctctgccttcccagcagccccactagggtctgtgacagtgaagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatgctcaggagtgagtgctcagaaaggggaaaaggggctgggactgaatccagatggactctattnnnnnnnnnnnnnnnnnnnnnnnnnnnnnnnnnnnnnnnnnnnnnnnnnnnnnnnnnnnnnnnnnnnnnnnnnnnnnnnnnnnccaacctttggagttggtctttagtccctcagacttttccgcccccttacttgcctttaagcagaactcccttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagaaaaaaaacttgatgaagttaaactgaagacaaataaaataagctactcctttacaaataagattacagattatcctaaaatatcaccggctgaccatacagttacatacttctcgagtgatcaagccacacgggagctcacagactcaaagggtgacatcatggagatcagcctgccctcccacagtgggcacagcatcccaggctttctgatttgcagtgctgagctgatgtggcaattttcctattcctgccccctgcagatttatgagctgtataatggtggctcagtgcccgtgacatatgaggtccagaccgatgtcctgtcacaggttcaggaaaaaaattttgatcaccccatcttttgctgcctcaaccccaaaggggagatccagccaggcagcactgcccgggtcttgtggatcttctcacctatcgaggccaagacctacacggtgagcagcaacctggatgacgtgggaggtggggtggtgctggcttcctcagcaggcctccctctgaccagtccctacccccaccctcctaactccccaggtggacgtgcccatacacatcctgggatggaactcggccctcatccacttccagggagtgggctacaacccccatatgatgggggacacagccccattccacaacatctcctcgtgggacaacagttccatacactctaggctggtggtgcctggacaggtggggaaaggggagctaggaaggcctgaggccttgtgggagagcctcagagcccagggtggccagacagaaggatggggaggctgggagggacatgagatgtagttgaggagggacagaggcagagatgagaccttggaactcaccctcctccagattcaggaggcagcaccagctccatgagaggtctaccctgccacctagtgcccacaggtgttcctgctcaagccgctccacctgcctcagggccctccatcccaccctcttcactgggatgaggatgttaagagatggcataggagtgggggtgttgaaaggtgtcaaaagggccaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggttcttatttctttacctccgctctccacccacagaatgtcttcctgtcccagtctcatatttccctgggaaacatacctgtgcagagcaagtgcagccgcctgctcttcctcaacaacatctccaagaacgaggaaattgccttctcctggcagccaagtcctctagattttggggaggtgagagtcccctgagccctggaacctccatcagagctccagacccatccaaccccacctgacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccgtgccatcctatccaattccatccctgtgaatctccagagactccttccccaaatcaacaggtgtctgtgagtcccatgataggggtggtggctcctgaagagacggtcccatttgtggtgaccttgagggcctctgtgcatgccagcttctacagtgcagacctggtatgcaaggtaggaaccagccccaggggtaaagtgggatggaggcatggctcccaatgctggggtggggtgtgatgggctgtgagatccccagaccccagcaaaaggcagaagtcagcccttggaagggagagggctcacagcataggccccgggcaagctccagaccttggccttctctctctctgtcctgggccatagctgtactcgcagcagctcatgaggcagtatcacaaggagctgcaggagtggaaggacgagaaggtgcggcaggaagtggagttcaccatcaccgacatgaaagtgaaggtgaggagggctgagtagatctcaaacacccagatctttctgactggccttaccagaggggacagaggtagtgggccaaggtggccctccaggccaaaccagccagcctgccctaagagattacatttcattcactggactcagcttagcatctccacagccatccggagaggctctctgggtagagaaggctggctgtccattccctgcctcgtgnnnnnnnnnnnnnnnnnnnnnnnnnnnnnnnnnnnnnnnnngagatttaggggagacaagcagagctcacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntagagttcaccttctggtggaagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtctaaaaatactttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgttttgggagggggagggtctgataataaacacgagacaacacttccttcttggttcctcccccaatttcacaaccttcctacactggtattcttgctcctcaatcaccttggtgtctgccacactgaacccccagagactcactgctcccctctgaagctgtggcccaactgtggatacaaattacttcctaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaaaaacaacaacaacaaaaaaaactcttcttgggggcatgagggaaggaatttctgaattaaatcttaatgagggagactgatttctcatctcactgaaccacaaagctccagggcacgagggcaagctcccttggagctgcccgcctacatttgatggcatttccttcactcaggaaaccagaatttcctggtctgcaggcagcacttctctagggaaggttctagaggaaaatgtgactgggaattgggcaggaccaagagggcagcaagaggatcaaaatgggggagtctctttagcctctcacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggcctcttgtatcttaagcaaggatctctctttttccacagaagagaacatgctgcacagcctgtgaacctgcgaggaagtacaaggtgaggcggcgagccccaggcccatcttcttgggacaacttgggagggtccttgcttgctcccagagagacctccaacccctggaccagactcccagaaaggagggatgaaagtgccaatgagtggggtcacacgcttatgtggcccaaagagcctgtgggggaactaggtcaagggaccccattggtgatgtgaatgtggtgcccaaacccttgtcatccatgtccctcccttcccaaagacactgcctcccatcaagaaccagcagtctgtcagccggcctgccagctggaaactgcagaccccaaaggaggaggtgtcctggccctgcccccagccaccctcgccaggcatgctctgcctgggccttactgcccgagcccatgccaccgactactttctggctaacttcttctcagagtttccctgccactttttgcaccggtaagctcttgggaaggcaggggtcaccttcctcttctctctgtcccctcttccactccagagcaggggctgcccagacccaacccatggtgaggggtggagcatgggcagcagcagtggggctcattgagcgcctctgggcccaggatgccccattactgcttaggcccttttaggagggggacaaagcagccagagccccctggggaacacacctaggcagcccctacccctctttgttccttctttttccaactgggagtctctctagctcccgtgctaactctgagggtttgagcttggttctctgaacccagtagcactacatttgggttaaggctgggagtgcctcaaactcatctgtgtcctcatcactgggcagctaccaccactgcttgtgcccccaaccccagcatggtggcttgcccaggccactcagaatgtcatggctgccacagggagctgccaaagaggaaggcccccagggaagagtcagagacttctgaggaaaaatcccctaacaagtggggccctgtttccaagcagaagaagcagctcctggttgacattctcaccacaataatcaggtaccagctcacaccctgggccctcattgccaccttcgcatccctccagctcattcagccagagccccttacattccagcactcttcctttcttgagaaatagaacttggtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagagacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncagaacttctttgcaagatcccaccacggtccccccccccggcccccccaccccaggacagtggggctctgatggagctggctggagggattctcaggtctggatcctgagttctgccccattccagggctggcagatgagtctggagccagtccactatctccccagggttcctgcccatcatgtgtgtgaatctgacctgccctgcctccagcataccatgttcgcttctaaaaatccatgaggctcaaagatgtcttccttgaaagaagggacaggaggaacaactgaccaagggctcccctctccccacctagcagcccctttcccacctagaacctagctctgaagccagggctccccaggggcttcagggcccctcagcaagggccttctccttctccctgccacctaggggcctgctggaagacaagaacttccatgaggctgtggaccaaagcctggtggagcaggtgccgtacttccgccaattctggaatgagcagtcaactaagttcatggaccagaaaaacagcctgtacttaatgccaatcctgcctgtaccctccagcagctgggaggatgggaagggcaagcagccgaaggaagacagaccagagcactatccagggttgggaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaagtgagctgggcgggcatcgggcccacaccacagcctgagtcccaggagtccatgcaatggcagtggcaacagcagctgaatgtcatggtgaaggaggagcaagaacaggacgagaaggaggccatcagaaggtgagcagccaggccccagtgaggacgggagggaggcgaagtgcagaaggttcctaggaggcagtgggatgccaaggatggctgctgcctgtannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagaattgttatagtctgcaaagtcttgtaaatgggcttctagagccagaaaaggaccacctgtcttgcaggtgggacttcccgccggggctgccctacagatgatttatgtcattgtagggattccctaggccccacccagtcccttctgcgccctctcatcacttccctacctttctctaaacttggccctgagatgcctactacatcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcgagcgaggaggcgcgcgggcactgcggccgccctcatgctagcccccgccccctcgcaggctcccggccttcgccaacctgcaggaggcgctgctggagaacatgatccagaacatcctggtggaggcgagccgcggggaggtggtactcacctcgcggccacgcgtcatcgccctgccgccgttctgcgtgcccaggtaagggcgccgggaccagcggccgcgcagaccgcgcgagaggggccacgggggcagccaggctctgcccgaagcctcgcccccacgcctacctgggactttttgtcttccaggagtctgaccccggacacgctgctgccgacgcagcaagcagaggtactccacccggtggtgccacttcctaccgaccttccgtaaatgcccgcgcccagcctctccgacatgccgctaggggtcacgcctggccccctctccaccgccagtaaaagcatctagtcttttttctgtatctaccttgactttatttccccgacgagggggaacgcgcgcgcgagctctgagagggaaggatggcagggagcagcccgggcggactgcagaggcgcggcgggaacttagggcccgggaggatgcacctctccgcccacactgcagctggactgagacctggccgctagctgccggccgctggaccgggtctggccagtggagggcgctccggacggggcctggctggcctttgccccagcgcaccttggtgcacagtaactgagtgccaggcctggccagcgaagcaggtgtccactgggcagggcggccacccctcacctttccgcgccgcgcctgctcggaggcttagcacccccagggccagccccttagcatttgccgagctccgagtgcccaagaggctggggaggaagaactgaggcacgccgcggagggccaggaactaggctggggcgggtcgaggtcaccactggatttcgggccacagaccctgctaagcgactcccactgtgaatcagtcctcagacgaggatggatacccacctctcggttcctcctaacccccgcccgggagcttctgtcctctgcttctcggctcctccctcccagaggcaggtgggtggaaaagtgacatctggtgttgttccagaggcgcccagggtccccagcaccctcccctccgcccccgccaccaaggcctcggagaagctccggtctcagagcccgggcgggggaggggggagcgctccgcctccatgagtcaatatttgccccgagcaaacgggcgcctggacaggtgtgagtgtgtgtgtctgggggcggggactgaacaggcagtataagagcacacggagcccgggctccgggacaagctccaaggcgtgggctggggacgaagccaagctaggctgggccgggcggaggggccaggaaagccgggcggagggtggctgggaaaggaggggcgggccaggtgcgcacctgcggtggccgccgcggccgccgacgtgtcagccgcagatgcgttcaggtgagggcggaggctagcggggcgctgtgcagcactgagctcgcggaagaccaggaccaggagatcaccgagggcgaccgccaggccccgggccctccgctcccgccccgcgacgagcccctcgcacaaaccggacctgagcgttttgttcgttcggctcgcgtgaggcaggggcggcctctcagcaccagcccgggggccggcctgatcgccacgcaggcacctgccgccgccaccgccaccgccatctcaaccgtacgggtgggagaggctgtgcgccgctccaggggagatccggctcccatccggccccacccgccctgccttgccctgcccgcagcttctgggctgccaggctccattctgcgtcgtaatacgggctgagccggctccagcgggtggcgaagggggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctcccgcaggggctaggccctccccagcctcctgtcccggttcctgggctcctggtcatgctggggaagatgcaggccctcacggagttggagccaaggctgaggtgtggaaaatctgggcgggggaagctgagggccaccatggagcctcaaatccactcctcggatctctgtggtgctgctgacatcctgagatgtcactccctgacactcccttggtccctctttcagtctaaaaacgtctgagacagtcagactgactgaaaaggacaagcccccaggcacagcggttcaccaccttcctcaaacctcagaatcccacatcctcgcttccatcagctggggtggggtaggtaggtggccctcagctcaggcagaggcagccccgggagatgggaaccagggtcagaagtccctccaaactgcatgttcctagaggtgctggattgtcccctccaccaccacccgctctagtgcccctatgtcacccaactacccagaggccccaaaggatctgctgagcccgagtccgtggcacagagagagaagggggaggggcgagatggcgttggacagatcccaggcaggtttagggagagggtggactacagcagggctcttcgtggagcagccccaggggcaggggaaggtccttgggtcctgggctagggtgtgtgcagccctctcctggcccccctgaccaggctctgtcctgggccagccctttactctgcctggcactgaaagatttgcctgtcaggggcctggcatggtcctggtgcaccttccctgtgtctttgagggcttccccttccctccccaaccatgtcaggggccaggattccacatcctgggtttgcacaacaaagagatctctgcttggaatgaccccctcccaannnnnnnnnnnnnnnnnnnnnnnnnnnnnnnnnnngccttgaggggagggtggtgagaaggaggcagccacccagtgggcggggatctttcctggtgactgagaattactgccccttcaccccagggcctaatttccccagtcccccacccactatccatcctaagactgcagttgctggccattaccagggatctggcctctcatccaggtccctcctccgctgctccgctgccacagggcggggtctcccagtgccgggcaggcctgcaggtgcggggctgcatggggaggggggcactcagcagctgctgtacgaggcaggccccctccccctcctgctcaaagctggagcctgcttcctgtcgtccctgtcagcaccctgggtgggggagggaccaggtagtgggggaagtggaaaagggattgagcggttggagtgcagcagctgagaaacagcagaagagaaatggagaaggatgagaaagagaccaagagcataggctgaaggaaagaggggtgtgagaacacaggggagatcccaggggctgcagaggctgcagaccctagactgtgagagcgagaccagaggcagagatgaccagagagaaaccagggaagaggaacagagagagacacatggagacaattcaagataacaagagatgtacgctgagagagattggggatgtggattcatggggctagggctcaggggaccagaggcgggaggcatcgtgtgtatggagaaagggggaagctgcaaaacagccccatcccaccccaaactagggtgggaattagccccagcaaaggataaagaggtgatttctaggtggcagacaggtttggaggtggggggagggtgatacccttatcttggaagcccatctcctcccattaaaggacagaatgtgagggaggggcaaggcccaggccgtggctggacaccaggggccagtgaggggcacagatctgactccatctctctgagtttccctcttggccacagatggacaagccaacagctatggcagctgccctgcccccacccaccccaattattctggcctagccttggaatttctgggcttagaggccgcccagggtctgtgtgacagtggagggacctgggagtggaggcactggctggcaggggggaggctcacacccaggtgctaatccccatccggccaaggcagtaccagagcctcactgaccttcttggagattagtccccatggagaccatggcaactcgccccctcattctggctaggccagacgggtgggtgggtggggcaggcatacgctggatagggaggggaaggaaccttccagaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcggctcggttcccaagcattttttcctagagagcagaggcctgagcaagaacaaaggaaattggcacactcctctctgaccctcccaaccttcccaaactgaggtcatgaactgaggagtgattcaaatagggtgaggcaggaaggcgctccgtcctgagcactaggctggcttctgccaggaaccagggtgagaaggagggtacagctcccagagccatgcttgcagcctgaccacccaccaaccaggcgagcgccaggggtggtgggaagcaggtgtgtcagagccagttataaccacaaccacagcaacagcggcggcggnnnnnnnnnnnnnnnnnnnnnnnnnnnnnnnnnnnnnnnnnnnnnnnnnnnnnnnnnnnnnnnnntcttaacccactctcttcacccttcggtgtctgtatgaccttgcgtgggtggctgagctgatgccttcctgggtgactgtgaagtgggtatgtgcacatgtgaaagtgatgggacatattcgtgtgtgagtnnnnnnnnnnnnnnnnnnnnnnnnnnnnnnnnnnnnnnnnnnnnnnnnnnnnnnnnnnnnnnnngaccaagtgtctggccatacatgaatatgacattgaggtttctatggaatcctagaatgagactattcacatgcgaagcttgcgggatcacatctatgtgcctggaactgtggatgattctgtctccatctgactgaacgtgtctgtgtgtgccctctccctgtcaccctatgtcacccctgtggctttgtgtctgggtgtgtgagtgatggtgaaagctgcctgccagctcctggatggtggccagttctctaaccccactgaggggcagaggagaggggctctgtttctagtgggatagaacaatgggtctgtgtgggtgggactcctaactggcccaaactgggctggtctctgtaccccgttatctctgtctcccaggagaccagtcctgcacagccccagggaaggtgctcatctgctgattctgggctccagactgggggtgggggcgggggccagagaagcaggggtggaaaggaggggctggtgatggtggcagggtgagggaaggcacccttccctgaatttgcatcttcagtacccaacccctggatgatttcccagtaaaacgaggggggagggctgggaggcgacccgctcgaggaggggacattggccaccagcccctctccgctgcccagcagtactgagcagtcaggctgttcctggggtggggctctaggtgagagaggtgccacttctttgggattctcagggcctttgcctaggacggagattgggcaggagcttaggagtgggtttgagggcttccttgcgggatctatgtgtccccacatgggatgggagtccgggtgggatgtgagggcctcatggaggggacttaggctcaaaggattctctgtgggatcttatgggtctgtatgggatgggggtttcggtgaaatcagagggcaggagatggtggctagggatggggtgaggtcctgaggatttccttgtgggactcaaaggtcccagtgtgagatggagaactgaatgaggtcagggtgggggccctgagggtgataagatctctgtgagattttgagggctctgtgaagtgtccactggggtttctggtttttgtctacctaattccccgctccatgccccaaggccaggctgtggatgtggagggggtaggggtgactgtgctgagctggcaaacaaagccacctgggcacccatccttcaggaagctctgggcgcaactgatctgggaaaacaaacaggaacagctgctaggggtggggggggtgggcggcatggcagctggggagaggagcagagctggaccagcctggtagggaaaggaggagaccagtagggggtccgtggtacccaaggggggttatcgatcccagagtcctctcctggcacagtaggcagggctgcctcgaagccccttcccctcagtggttcttctctttggcgataactgtgaggcctaaagtcaggaggggcctgagggcgttaggggaaccctgagatgtttagaggtcttctgggccagtgactatgggtggcggtggtgggtatgcccatttctgagagagctggtcgcagttactagaactagaagtcatgggtgagggggtggggggggtacctccataccatttccacggggagggacaaggtagagaaggggagaggctggctgccgccaagaccagagtttcacactggaggaggagtaggggcccttctcacacacggggtccactccccaactccaagcgggactcctaattctaacctggacctcggctggacggcctccccgcttttcctcctcccctaaccctggaaaaggcgcgacgcaccactcctgcagaatgccgccgtgggacccacgcgtggagcccgaccctcgagagtacgtccagcccccagccccgctcggtgcacagtgtccagtctccgacagacacccccacaccctctgaccaacacttggcgtccgcgtcagggacagcgcagacgcacagcctgacgcactagccacccgcgcccgcggcccctcccgacctggacctccccttccaccccgccctccgcgccagctttcgggccagcgcggcctgctcctgccctctggtggccgaatccagaattgtcggatagagatgggggaaggagggaaggcgagatctgattcttcacccctcacccctgcccgggctggtgacactgaaggcaaagactgggacaccaagggtccagaactggctcgtgccccactctgtggtttgtattccctccttgaggtttgtgtttgttcaaacccccgctaggtagggaatcaggacactgttctgacctcacccccagtcaaacctgcctgaggttgtcccatctctgacccatatcctcctcactcggactctaccgccatgcccagttctagggccctgtactgttggcacccatcttcacacacactgcgagctccaacagtaactcctcaccagggaaaagagagcccaagggatcctctgtctgcccccagctcctgctctcagcaacccagaacttcagaggtgggagagcaagctgccaagacccccccgccaacttccattggagaattttctctacaccccttttcccgtggacatttggcccacgcctgaccaccttgttcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaggcttctgggagctggggtgtccgcagctggcctgtgagcactttttggcacgggagcttcaatggacagcggcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcatggaagcaacaaaatattagctcccttctcactcgcccctctcacctctgctggaatatccccacccgcacccaccaccattaaggaaaacctacaattttgcaagaagcctttgtgtctcatttgctgtggttagaaaattccactgcaagtggtgaggaaatttcctacctggagctaccccttgggactcccaacctttttggagctcccaaccttttcctcttccccctctacctccccttttctctacacnnnnnnnnnnnnnnnnnnnngccgccttttcccagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgggagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctcagctgctaactctcaggcagacgtcgaggctggaacccttagagcctggagctttgtgggagacccgcagcggcaccggttgagccgagctaaagcccaacattcttcctacctcccgcggcgagccactccctaggtggctattatgcctaaacactgagtaaaccgcgcggccctcgggcggtcgtttccagcgcccggcgccgccgcgtggcgagtcctgaccaccgcctcctctccttcccgggcccgaggacaccccgggggcgcggcgcaggatctgggaacgcacgcttgggaggaaaagggccgcgatggaccgatcccccaggaaaaagaggactgagcacatgaagcgaatcctttgggcagagccctctctacctccgccttagcatcgcccaaaagagaggcctggactagggccgaggaaggctggcgattagactctgggaagaacttccgctcagcgagtcagcagcgcggtcttagcccgaggcccaggggaggccaggtcccttcgtccccgagggccgggacagggagggcgcgttctgccccgaggcctggcgtgccctgcctggctcaggctggaaagaaagaaagcgcggagtcagcctgggctgcggcgggcgcggggtgcgcgcctcagcactttctgttgtctttcccgcgggctggggccacagcggcggctgcgggagaggcctcggctccccagcggagaccagagccgaccgcagcctgcctgctgactcagcgaccccacccccgagggaaagggtgcggacagggggacaaggcccaggtataaagctcggcccgcccggccgcgcgcagcctcgcagccggtccatttccctcgcgtgccgcgctcacccacccggtgaatgccctctgctggggtggggaagggaggggagcgcgggaagactgcgagcccctggcagccgggtccagagctaggccgggtccagcccccgggttcttcatccgctacccaggttgggcaggctgtgtgtggctcgaaacggggctcttgagacgctgagcgcgtggtgccacccttcctgtccgcagcggcatgagcannnnnnnnnnnnnnnnnnnnnnnnnnnnnagcctcacgctctgggacgaggaggacttccagggccgtcgctgtcggctgctaagcgactgtgcgaacgtctgcgagcgcggaggcctgcccagggtgcgctcggtcaaggtggaaaacggcgtgtgagtgattgcctggccgcggctgggctgggttgaggggcgctgggttccaccggcctaggctcagagcctcagccccctgtcctgtgagccctggagagacctgccaacatgggaaaccccatagcctccccaaaattccccacccatcgcaagataaatcatgggaaactgtgtatttttgtgctagctaaagagacacaggcgagaagcgaccccttgagggtccctatgggaccccaccgtgaaggggtttctagtgtacttacacccttcccagagtttttctctgtgcaaaattagaaggctctgcacttggcttaacaaaagaaacccaatccctttccctggccccaccctggtctcccagagcccagcttttcccaccctgccgccccctacccctgccccagctgaggtcccagtttctgctgtgatattcgggaggggtcctccaatgtgtccccatgcaaaggcctaaacccttgaggcaggagcactgggtcgggagatcttcataaccaggaaaactctttcccaacctggggtccttgagggggaagcctgggcgcctcaaaccccagaaagcagcaggttttggacctttagttccgagaggggctggattgccatacaatgggggtgtcaggggaagggtttggggttggagaaaaacattgtgcttctcagtcttttcaatcatggccggcaagggggagggcagagagggtgcggctggggcagatacctcccagctcttctccttttccagttgggtggcctttgagtaccccgacttccagggacagcagttcattctggagaagggagactatcctcgctggagcgcctggagtggcagcagcagccacaacagcaaccagctgctgtccttccggccagtgctctgcgcggtgagcacagcccacttctcctcccctgcctcctagctccaggccctggcctcaccatggcacctagaaaggctgttcaagccctgcatggggtgtgtgtaaacaaactccgagatgggtggtagtagcattatttctcagaatcagaagggtgtctccaagaaagttgggattgtgggcgtaggcccggagcccagaaatcatcaccacggacagcgctgaggccatcccagtcctgagtgcccagggttcccccacattccatcgtccacacgtgtgagtgagtgcctctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaaagagtcagtctcagtgtgtttgtgtgtgtgttattgtgtgtgtgagggtgttgttaatgaatcaaatacaccgaaagagaggacctcattttccatttgtgccgacctggttactagtagggctgggaagtggagagcagaggacccagcccaggccagcccaattcttctgttccaagaggctgaagaaccctgcagtgggaaggccggagccaagatgcaatcagtattgatgccctgacatgtctattctgattctgccacccagtacctcttagctgggcactaatctgtgagaggctgtgcgaaggggctgggcaagggcttgggtagttagtcaatgaggacctcagggaactgtcttggtgaatgggacagatttctctaaggacctctgattttgctcctgcaaaatgggcccatgtcacaagatgctctctagtcctgacctgaaagggtctctggaaatcctatcccccaatcacctccttatctcaaagtcatgggaggggccaggttcgggggaacctctgagcgatccatttcccagactgccagggagctgatggggtgagctctgggagtgcctgtgggtcctagacagcttcacagcttgtgaggtgcagtgggtgggggaagtgaggcagaggatgtatccgttaaccctttggaagccctgatctttctgtcctgatcttgctgtgcctcagaaccacaatgacagccgtgtgacactgtttgagggggacaacttccaaggctgcaagtttgacctcgttgatgactacccatccctgccctccatgggctgggccagcaaggatgtgggttccctcaaagtcagctccggagcgtgagtcctggaactggaaccagggagacagaagagcagggaggcaatggggaccaaggaagagggtggagaaaagtgatgtggggagggcgctaagagagatattggggacttggaggcaaggggagggagatgatggagtcagactggaagggaggaaatgaggaggtcctaggagaatggggggcagcgggcaatatgggcacagatgaagggctggagaacatgagggggtggaggaaataaggggtgggtatagaagactggatgagaacggtgacagaaagactggaagcagtgggcaggggagggagagagccacggagggaaagttgaagcaatgattttggaactgggggttgggagctacaaaaggggggctgctggtggagaacatagggtgggggtgggggcagagcttactccctgcctctgtaccctcactggcctaggtgggtggcctaccagtacccaggctaccgaggctaccagtatgtgttggagcgggaccggcacagcggagagttctgtacttacggtgagctcggcacacaggcccacactgggcagctgcagtccatccggagagtccagcactaggctccacggccccagacaccttccctgaggacactcaataaaggttcctgaatcttcctgcccctcttgcctccttggtgtcttgctttgtgatctccatcctcatctttctgaaggtccctccctgccaagctgaccctccccacaggctccagcatccttggctacgaagtgtaagctggctgaggttgtgtgtgggtaggctctggtgggtctgacagcaaagtgctggtaagaatcttagcaattctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaccttagtaattctgttaaatggcaccagtcccaaacctctccttggtaaggtccaccactatgggccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccttaccatgcactgaagttctgggcttcctctgtaaccccagagcattttcaacctttatcttcacccagttccctgcccagtaagggaagcctgagcctcctgcgttgtcccatttcctccattcgcaggaggggagttagtgggaaaccccactgcttcccagggccttcaaaggggagacttccaccctctgagcccagcccccagaccctttgaacaatgacaatcccttgcagtggtgttgaactccagcatttacaaagcatcttcatatcccccctctcagctgctccataccaccccctcttcactcttcctttccctccctacccttcagaaaactggtgagctgtagaagtcacccctcagttgctaagcctgggttctgggcaagtggcaggtgcccataggcatggcagggctgagtatggtggcagccattagagcctctggcactagtatgggttttttcccagttccctcttcggtccagttggtgatgccccccaacccccacacagacagagcctctcaagattcttgggactgccttcatcctgtccttcagggattcttgtgttttcaagtccccagtggacctatcagtggggaagggtggctctaattattgccccaggccaggcagagcaggacaatgccagggagcagtcagacttgggcctgaaatccccagaggttcagctgtgcaaagtcaggcagctgctcctttgggaagagctccggaccataagtcagaacacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtttgtggtgttatggaaaagcactggggtagtcaatgtagggacctccttcccctacctccccgctatggatcctagggttgggtcaggggaccaagggagtgagctttggatggcgtataaattttgctttacactcgaatgggctctagagtccatggcccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggtggaggctgaactgagctgtggctaaaagccacaggcctcagcagagccccaaacttctgtgggccccagaggaacctggggttcctggctggtcatgggcacctccccatccccttgtggaatggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcaggccacttccccactgcttgctttggggagcaggcaatgagcaaagggagatctggaatctgcccttgactgagctgctctccctacaggggtatctgtggttgctgggaagaatcagcaagatggcttcaggaactagaagtgtgactctggaccttctggtcctccattcattctgggcaggctggccatctgcagaaccacatgggaccagcgctcagcagggtaggaaggggatgaaggggaaactgccaaggactctcctgttggcttccctagctgtacttgcagcaggaggggatggagaagatcaagactcaggtgggtggagatgactgccccccatgcacacatttcctggtgctccaccccctcagcaaatgacttctgggtaagactgagggataaagcctatactgttgatccttgcccagctcacagctgggatatttgggtagacctcactaggaccctctcctttacaccttagcctcccactgagagcattaagtgggcgaattttgatgcctcccagctttcccagctcttacctcctgcccctggctattttctgttcttacctcctgctccttgttttctgtccatagtgggtgggggaagcagaaagaggaatgagaaggagagggattgggatcttgaccccaacttcaaggccctcaccttctgtggagccctgagtttggtccacagggaacattccaacctctagtctttgctcccaccagattccaggtccacatcctgaaggcttcatccctgtcccactaggatcctcccaggccctgggatccctctttttccctccacactctgcctcccactcttctccctggctcctttgcaccctccacgagcaaagatgaacactccagaagttgcagcaaatgagggtgaacatgggccctgtagaaccccagaagtaggagtttccagccatcctcaatcaggcatcccaggaccaggcacttagcaggagacctgcccattcccaggcctcctccacccctgggctcaagcagtgagagaggactttgagaatgccagttttgacagtacccgttccatactgcaaggctgcagaaggtgggcttctgggctggagcctcatcttcagccagcatcctccttagcctgcctctccagagccacgaagccccataggtacccagagagactccaggccacttgggaaccacatcaacatctttgcatgagttagaaataggaatccttttcttggagtggattccagccttgtacagtgcacaaccctgtcttaacccctccacctctccagcctcgttcagggatagccagaatgggagtagtggtgagggtgggtggcagactgggaggacaggggatggggaaggatgggagcagaatagttgtacagggaagtagcagggggctcaggaacccatcagggaaagccaggacagattctcaacatctttagaggccttcagcagtccaagccactactaatttatggggactgaatgaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngccacccatcataagttgccaggagagatggcccagcaccaggcctgaggcttgggggccccagaaaggcaggaaacgggccaaagcaagaaggagacggggaaggggttcgggtctctagggaagagcagagagtgggagaagaggaggagggagcggggatgcgggcagagataaagggagcaacggctgcagggccgggcgcgcagcgacaggcgagagggagggaagcggaaatttaaaagtgaagatgcagataacgcagcctggagacgggaacccgggtggggatagagggagggagggggaggagagtgagcggcgagtggagcgggagccgactggaagaagggctctagggagggggctgtggctgctggggtccgaggtggggccgggtacaccagccccatcactgtttgcagagagtcagggaggcggaaaagacacgcgctctaggctcccatcagggcacatggcccgggcccatcccccgcgcgtctccccggctgcggggcgcggggggctgccgggtgcgcttggctgtggcgcggcgcgttggagactttattgcgatgggacgataagaggggcgggggcggggtcctgggggccgaggcggcagcgct